(19) United States
(12) Patent Application Publication
(10) Pub. No.: US 2002/0019020 A1
(43) Pub. Date: Feb. 14, 2002

(54) METHODS FOR TREATING CARDIOVASCULAR DISORDERS

(76) Inventors: KENNETH RHODES, NESHANIC STATION, NJ (US); WENQIAN AN, FRAMINGHAM, MA (US)

Correspondence Address: LAHIVE & COCKFIELD, 28 STATE STREET, BOSTON, MA, 02109, US

(21) Appl. No.: 09/350,874

(22) Filed: Jul. 9, 1999

Related U.S. Application Data
(60) Provisional application No. 60/110,033, filed on Nov. 25, 1998.
(60) Provisional application No. 60/109,333, filed on Nov. 20, 1998.
(60) Provisional application No. 60/110,277, filed on Nov. 30, 1998.

(51) Int. Cl. C12Q 1/68; G01N 33/53; G01N 33/537; G01N 33/543; C12P 21/06

(52) U.S. Cl. 435/7.93; 435/7.1; 435/6

(57) ABSTRACT

The invention provides methods for identifying compounds suitable for treating a cardiovascular disorder, as well as methods for treating a cardiovascular disorder. The invention also provides methods for determining if a subject is at risk for a cardiovascular disorder.

### RELATED APPLICATIONS

This application claims priority to U.S. provisional Application No. 60/110,033, filed on Nov. 25, 1998, U.S. provisional Application No. 60/109,333, filed on Nov. 20, 1998, U.S. provisional Application No. 60/110,277, filed on Nov. 30, 1998, and U.S. patent application Ser. No. 09/298,731, filed on Apr. 23, 1999, incorporated herein in their entirety by this reference.

### BACKGROUND OF THE INVENTION

Mammalian cell membranes are important to the structural integrity and activity of many cells and tissues. Of particular interest in membrane physiology is the study of transmembrane ion channels which act to directly control a variety of pharmacological, physiological, and cellular processes. Numerous ion channels have been identified including calcium, sodium, and potassium channels, each of which have been investigated to determine their roles in vertebrate and insect cells.

Because of their involvement in maintaining normal cellular homeostasis, much attention has been given to potassium channels. A number of these potassium channels open in response to changes in the cell membrane potential. Many voltage-gated potassium channels have been identified and characterized by their electrophysiological and pharmacological properties. Potassium currents are more diverse than sodium or calcium currents and are further involved in determining the response of a cell to external stimuli.

The diversity of potassium channels and their important physiological role highlights their potential as targets for developing therapeutic agents for various diseases. One of the best characterized classes of potassium channels are the voltage-gated potassium channels. The prototypical member of this class is the protein encoded by the Shaker gene in Drosophila melanogaster. Proteins of the Shal or Kv4 family are a type of voltage-gated potassium channels that underlies many of the native A type currents that have been recorded from different primary cells. Kv4 channels have a major role in the repolarization of cardiac action potentials. In neurons, Kv4 channels and the A currents they may comprise play an important role in modulation of firing rate, action potential initiation and in controlling dendritic responses to synaptic inputs.

The Kv family of channels includes, among others: (1) the delayed-rectifier potassium channels, which repolarize the membrane after each action potential to prepare the cell to fire again; and (2) the rapidly inactivating (A-type) potassium channels, which are active predominantly at subthreshold voltages and and act to reduce the rate at which excitable cells reach firing threshold. In addition to being critical for action potential conduction, Kv channels also control the response to depolarizing, e.g., synaptic, inputs and play a role in neurotransmitter release. As a result of these activities, voltage-gated potassium channels are key regulators of neuronal excitability (Hille B., Ionic Channels of Excitable Membranes, Second Edition, Sunderland, Mass.: Sinauer, (1992)).

There is tremendous structural and functional diversity within the Kv potassium channel superfamily. This diversity is generated both by the existence of multiple genes and by alternative splicing of RNA transcripts produced from the same gene. Nonetheless, the amino acid sequences of the known Kv potassium channels show high similarity. All appear to be comprised of four, pore forming α-subunits and some are known to have four cytoplasmic (β-subunit) polypeptides (Jan L. Y. et al. (1990) Trends Neurosci 13:415-419, and Pongs, O. et al. (1995) Sem Neurosci. 7:137-146). The known Kv channel α-subunits fall into four sub-families named for their homology to channels first isolated from Drosophila: the Kv1, or Shaker-related subfamily; the Kv2, or Shab-related subfamily; the Kv3, or Shaw-related subfamily; and the Kv4, or Shal-related subfamily. Kv4.2 and Kv4.3 are examples of Kv channel α-subunits of the Shal-related subfamily. Kv4.3 has a unique neuroanatomical distribution in that its mRNA is highly expressed in brainstem monoaminergic and forebrain cholinergic neurons, where it is involved in the release of the neurotransmitters dopamine, norepinephrine, serotonin, and acetylcholine. This channel is also highly expressed in cortical pyramidal cells and in interneurons. (Serdio P. et al. (1996) J. Neurophys 75:2174-2179). Interestingly, the Kv4.3 polypeptide is highly expressed in neurons which express the corresponding mRNA. The Kv4.3 polypeptide is expressed in the somatodendritic membranes of these cells, where it is thought to contribute to the rapidly inactivating K+ conductance. Kv4.2 mRNA is widely expressed in brain, and the corresponding polypeptide also appears to be concentrated in somatodendritic membranes where it also contributes to the rapidly inactivating K⁺ conductance (Sheng et al.(1992) Neuron 9:271-84). These somatodendritic A-type Kv channels, like Kv4.2 and Kv4.3 are likely involved in processes which underlie learning and memory, such as integration of sub-threshold synaptic responses and the conductance of back-propagating action potentials (Hoffinan D. A. et al. (1997) Nature 387:869-875).

Thus, proteins which interact with and modulate the activity of potassium channel proteins e.g., potassium channels having a Kv4.2 or Kv4.3 subunit, provide novel molecular targets to modulate neuronal excitability, e.g., action potential conduction, somatodendritic excitability and neurotransmitter release, in cells expressing these channels. In addition, detection of genetic lesions in the gene encoding these proteins could be used to diagnose and treat cardiovascular disorders such as heart failure, hypertension, atrial fibrillation, dilated cardiomyopathy, idiopathic cardiomyopathy, or angina.

### SUMMARY OF THE INVENTION

The present invention is based, at least in part, on the discovery of novel nucleic acid molecules which encode gene products that interact with potassium channel proteins or possess substantial homology to the gene products of the invention that interact with potassium channel proteins (paralogs). Potassium channel proteins are, for example, potassium channels having a Kv4.2 or Kv4.3 subunit. The nucleic acid molecules of the invention and their gene products are referred to herein as “Potassium Channel Interacting Proteins”, “PCIP”, or “KChIP” nucleic acid and protein molecules. The PCIP molecules of the present invention are useful as modulating agents to regulate a variety of cellular processes, in particular, cardiac cell processes.

Accordingly, in one aspect, this invention provides a method for identifying a compound suitable for treating a cardiovascular disorder, e.g., arteriosclerosis, ischemia reperfusion injury, restenosis, arterial inflammation, vascular wall remodeling, ventricular remodeling, rapid ventricular pacing, coronary microembolism, tachycardia, bradycardia, pressure overload, aortic bending, coronary artery ligation, vascular heart disease, atrial fibrilation or congestive heart failure, by contacting a PCIP polypeptide or a fragment thereof, or a cell expressing a PCIP polypeptide or a fragment thereof with a test compound and determining whether the PCIP polypeptide or fragment thereof binds to the test compound, thereby identifying a compound suitable for treating a cardiovascular disorder. In a preferred embodiment, the binding of the test compound to the PCIP polypeptide or fragment thereof is detected by direct detection of test compound/polypeptide binding. In another embodiment, the binding of the test compound to the PCIP polypeptide or fragment thereof is detected by using a competition binding assay. In yet another embodiment, the binding of the test compound to the PCIP polypeptide or fragment thereof is detected by using an assay for PCIP activity.

In another aspect, the invention features a method for identifying a compound suitable for treating a cardiovascular disorder, e.g., arteriosclerosis, ischemia reperfusion injury, restenosis, arterial inflammation, vascular wall remodeling, ventricular remodeling, rapid ventricular pacing, coronary microembolism, tachycardia, bradycardia, pressure overload, aortic bending, coronary artery ligation, vascular heart disease, atrial fibrilation or congestive heart failure, by incubating a cell expressing a potassium channel comprising a Kv4.3 or Kv4.2 subunit, or a fragment of a potassium channel comprising a Kv4.3 or Kv4.2 subunit, and a PCIP polypeptide or fragment thereof, in the presence and absence of a candidate compound; and determining whether the presence of the candidate compound modulates the interaction of the potassium channel or fragment thereof with the PCIP polypeptide or fragment thereof, thereby identifying a compound suitable for treating a cardiovascular disorder.

In yet another aspect, the invention features a method for treating a cardiovascular disorder by contacting a potassium channel with an effective amount of a compound that modulates the binding of a PCIP protein to the potassium channel.

In a further aspect, the invention features a method for determining if a subject is at risk for a cardiovascular disorder by detecting, in a sample of cells from the subject an alteration in a PCIP gene which causes a mutated PCIP polypeptide to be produced, an alteration in a PCIP gene which causes abnormal expression of a PCIP polypeptide, or an alteration in a PCIP gene which causes abnormal processing of a PCIP polypeptide.

In another aspect, the invention features a method for identifying a subject suffering from a cardiovascular disorder by detecting, in a sample of cells from the subject an alteration in a PCIP gene which causes a mutated PCIP polypeptide to be produced, an alteration in a PCIP gene which causes abnormal expression of a PCIP polypeptide, or an alteration in a PCIP gene which causes abnormal processing of a PCIP polypeptide.

In a preferred embodiment, the cardiovascular disorder is associated with an abnormal Iₜₒ current.

Other features and advantages of the invention will be apparent from the following detailed description and claims.

### BRIEF DESCRIPTION OF THE DRAWINGS

FIG. 1 depicts the cDNA sequence and predicted amino acid sequence of human 1v. The nucleotide sequence corresponds to nucleic acids 1 to 1463 of SEQ ID NO:1. The amino acid sequence corresponds to amino acids 1 to 216 of SEQ ID NO:2.

FIG. 2 depicts the cDNA sequence and predicted amino acid sequence of rat 1v. The nucleotide sequence corresponds to nucleic acids 1 to 1856 of SEQ ID NO:3. The amino acid sequence corresponds to amino acids 1 to 245 of SEQ ID NO:4.

FIG. 3 depicts the cDNA sequence and predicted amino acid sequence of mouse 1v. The nucleotide sequence corresponds to nucleic acids 1 to 1907 of SEQ ID NO:5. The amino acid sequence corresponds to amino acids 1 to 216 of SEQ ID NO:6.

FIG. 4 depicts the cDNA sequence and predicted amino acid sequence of rat 1vl. The nucleotide sequence corresponds to nucleic acids 1 to 1534 of SEQ ID NO:7. The amino acid sequence corresponds to amino acids 1 to 227 of SEQ ID NO:8.

FIG. 5 depicts the cDNA sequence and predicted amino acid sequence of mouse 1vl. The nucleotide sequence corresponds to nucleic acids 1 to 1540 of SEQ ID NO:9. The amino acid sequence corresponds to amino acids 1 to 227 of SEQ ID NO:10.

FIG. 6 depicts the cDNA sequence and predicted amino acid sequence of rat 1vn. The nucleotide sequence corresponds to nucleic acids 1 to 955 of SEQ ID NO:11. The amino acid sequence corresponds to amino acids 1 to 203 of SEQ ID NO:12.

FIG. 7 depicts the cDNA sequence and predicted amino acid sequence of human 9ql. The nucleotide sequence corresponds to nucleic acids 1 to 2009 of SEQ ID NO:13. The amino acid sequence corresponds to amino acids 1 to 270 of SEQ ID NO:14.

FIG. 8 depicts the cDNA sequence and predicted amino acid sequence of rat 9ql. The nucleotide sequence corresponds to nucleic acids 1 to 1247 of SEQ ID NO:15. The amino acid sequence corresponds to amino acids 1 to 257 of SEQ ID NO:16.

FIG. 9 depicts the cDNA sequence and predicted amino acid sequence of mouse 9ql. The nucleotide sequence corresponds to nucleic acids 1 to 2343 of SEQ ID NO:17. The amino acid sequence corresponds to amino acids 1 to 270 of SEQ ID NO:18.

FIG. 10 depicts the cDNA sequence and predicted amino acid sequence of human 9qm. The nucleotide sequence corresponds to nucleic acids 1 to 1955 of SEQ ID NO:19. The amino acid sequence corresponds to amino acids 1 to 252 of SEQ ID NO:20.

FIG. 11 depicts the cDNA sequence and predicted amino acid sequence of rat 9qm. The nucleotide sequence corresponds to nucleic acids 1 to 2300 of SEQ ID NO:21. The amino acid sequence corresponds to amino acids 1 to 252 of SEQ ID NO:22.

FIG. 12 depicts the cDNA sequence and predicted amino acid sequence of human 9qs. The nucleotide sequence corresponds to nucleic acids 1 to 1859 of SEQ ID NO:23. The amino acid sequence corresponds to amino acids 1 to 220 of SEQ ID NO:24.

FIG. 13 depicts the cDNA sequence and predicted amino acid sequence of monkey 9qs. The nucleotide sequence corresponds to nucleic acids 1 to 2191 of SEQ ID NO:25. The amino acid sequence corresponds to amino acids 1 to 220 of SEQ ID NO:26.

FIG. 14 depicts the cDNA sequence and predicted amino acid sequence of rat 9qc. The nucleotide sequence corresponds to nucleic acids 1 to 2057 of SEQ ID NO:27. The amino acid sequence corresponds to amino acids 1 to 252 of SEQ ID NO:28.

FIG. 15 depicts the cDNA sequence and predicted amino acid sequence of rat 8t. The nucleotide sequence corresponds to nucleic acids 1 to 1904 of SEQ ID NO:29. The amino acid sequence corresponds to amino acids 1 to 225 of SEQ ID NO:30.

FIG. 16 depicts the cDNA sequence and predicted amino acid sequence of human p19. The nucleotide sequence corresponds to nucleic acids 1 to 619 of SEQ ID NO:31. The amino acid sequence corresponds to amino acids 1 to 200 of SEQ ID NO:32.

FIG. 17 depicts the cDNA sequence and predicted amino acid sequence of rat p19 The nucleotide sequence corresponds to nucleic acids 1 to 442 of SEQ ID NO:33. The amino acid sequence corresponds to amino acids 1 to 109 of SEQ ID NO:34.

FIG. 18 depicts the cDNA sequence and predicted amino acid sequence of mouse p19. The nucleotide sequence corresponds to nucleic acids 1 to 2644 of SEQ ID NO:35. The amino acid sequence corresponds to amino acids 1 to 256 of SEQ ID NO:36.

FIG. 19 depicts the cDNA sequence and predicted amino acid sequence of human W28559. The nucleotide sequence corresponds to nucleic acids 1 to 380 of SEQ ID NO:37. The amino acid sequence corresponds to amino acids 1 to 126 of SEQ ID NO:38.

FIG. 20 depicts the cDNA sequence and predicted amino acid sequence of human P193. The nucleotide sequence corresponds to nucleic acids 1 to 2176 of SEQ ID NO:39. The amino acid sequence corresponds to amino acids 1 to 41 of SEQ ID NO:40.

FIG. 21 depicts a schematic representation of the rat 1v, the rat 9qm, and the mouse P19 proteins, aligned to indicate the conserved domains among these proteins.

FIG. 22 depicts the genomic DNA sequence of human 9q.

FIG. 22A depicts exon 1 and its flanking intron sequences (SEQ ID NO:46).

FIG. 22B depicts exons 2-11 and the flanking intron sequences (SEQ ID NO:47).

FIG. 23 depicts the cDNA sequence and predicted amino acid sequence of monkey KChIP4a. The nucleotide sequence corresponds to nucleic acids 1 to 2413 of SEQ ID NO:48. The amino acid sequence corresponds to amino acids 1 to 233 of SEQ ID NO:49.

FIG. 24 depicts the cDNA sequence and predicted amino acid sequence of monkey KChIP4b. The nucleotide sequence corresponds to nucleic acids 1 to 1591 of SEQ ID NO:50. The amino acid sequence corresponds to amino acids 1 to 233 of SEQ ID NO:51.

FIG. 25 depicts an alignment of KChIP4a, KchIP4b, 9ql, 1v, p19, and related human paralog (hsncspara) W28559. Amino acids identical to the consensus are shaded in black, conserved amino acids are shaded in gray.

FIG. 26 depicts the cDNA sequence and predicted amino acid sequence of rat 33b07. The nucleotide sequence corresponds to nucleic acids 1 to 2051 of SEQ ID NO:52. The amino acid sequence corresponds to amino acids 1 to 407 of SEQ ID NO:53.

FIG. 27 depicts the cDNA sequence and predicted amino acid sequence of human 33b07. The nucleotide sequence corresponds to nucleic acids 1 to 4148 of SEQ ID NO:54. The amino acid sequence corresponds to amino acids 1 to 414 of SEQ ID NO:55.

FIG. 28 depicts the cDNA sequence and predicted amino acid sequence of rat 1p. The nucleotide sequence corresponds to nucleic acids 1 to 2643 of SEQ ID NO:56. The amino acid sequence corresponds to amino acids 1 to 267 of SEQ ID NO:57.

FIG. 29 depicts the cDNA sequence and predicted amino acid sequence of rat 7s. The nucleotide sequence corresponds to nucleic acids 1 to 2929 of SEQ ID NO:58. The amino acid sequence corresponds to amino acids 1 to 270 of SEQ ID NO:59.

FIG. 30 depicts the cDNA sequence and predicted amino acid sequence of rat 29x. The nucleotide sequence corresponds to nucleic acids 1 to 1489 of SEQ ID NO:60. The amino acid sequence corresponds to amino acids 1 to 351 of SEQ ID NO:61.

FIG. 31 depicts the cDNA sequence of rat 25r. The nucleotide sequence corresponds to nucleic acids 1 to 1194 of SEQ ID NO:62.

FIG. 32 depicts the cDNA sequence and predicted amino acid sequence of rat 5p. The nucleotide sequence corresponds to nucleic acids 1 to 600 of SEQ ID NO:63. The amino acid sequence corresponds to amino acids 1 to 95 of SEQ ID NO:64.

FIG. 33 depicts the cDNA sequence and predicted amino acid sequence of rat 7q. The nucleotide sequence corresponds to nucleic acids 1 to 639 of SEQ ID NO:65. The amino acid sequence corresponds to amino acids 1 to 212 of SEQ ID NO:66.

FIG. 34 depicts the cDNA sequence and predicted amino acid sequence of rat 19r. The nucleotide sequence corresponds to nucleic acids 1 to 816 of SEQ ID NO:67. The amino acid sequence corresponds to amino acids 1 to 271 of SEQ ID NO:68.

FIG. 35 depicts the cDNA sequence and predicted amino acid sequence of monkey KChIP4c. The nucleotide sequence corresponds to nucleic acids 1 to 2263 of SEQ ID NO:69. The amino acid sequence corresponds to amino acids 1 to 229 of SEQ ID NO:70.

FIG. 36 depicts the cDNA sequence and predicted amino acid sequence of monkey KChIP4d. The nucleotide sequence corresponds to nucleic acids 1 to 2259 of SEQ ID NO:71. The amino acid sequence corresponds to amino acids 1 to 250 of SEQ ID NO:72.

FIG. 37 depicts an alignment of KChIP4a, KChIP4b, KChIP4c, and KChIP4d.

FIG. 38 depicts a graph showing the current traces from CHO cells which express Kv4.2 with or without KChIP2 (9ql). Cells are voltage clamped at −80 mV and stepped from −60 mV to +50 mV for 200 ms. Peak current amplitudes at the various test voltages are shown in the right panel. FIG. 38 further depicts a table showing the amplitude and kinetic effects of KChIP2 (9ql) on Kv4.2. KchIP2 expression alters the peak current amplitude, inactivation and recovery from inactivation time constants, and activation V_{½}.

FIG. 39 depicts a graph showing the current traces from CHO cells which express Kv4.2 with or without KChIP3 (p19). Cells are voltage clamped at −80 mV and stepped from −60 mV to +50 mV for 200 ms. Peak current amplitudes at the various test voltages are shown in the right panel. FIG. 39 further depicts a table showing the amplitude and kinetic effects of KchIP3 (p19) on Kv4.2. KchIP3 causes alterations in peak current and inactivation and recovery from inactivation time constants.

### DETAILED DESCRIPTION OF THE INVENTION

| | | |
|---|---|---|
| I. | Screening Assays | 16 |
| II. | Predictive Medicine | 23 |
| | 1. Diagnostic Assays | 23 |
| | 2. Prognostic Assays | 25 |
| | 3. Monitoring of Effects During Clinical Trials | 30 |
| III. | Methods of Treatment | 31 |
| | 1. Prophylactic Methods | 31 |
| | 2. Therapeutic Methods | 32 |
| | 3. Pharmacogenomics | 38 |

The present invention is based, at least in part, on the discovery of novel nucleic acid molecules which encode gene products that interact with potassium channel proteins or possess substantial homology to the gene products of the invention that interact with potassium channel proteins (paralogs). Potassium channel proteins are, for example, potassium channels having a Kv4.2 or Kv4.3 subunit. The nucleic acid molecules of the invention and their gene products are referred to herein as “Potassium Channel Interacting Proteins” “PCIP”, or “KChIP4” nucleic acid and protein molecules. The PCIP proteins of the present invention bind to and modulate a potassium channel mediated activity in a cell, e.g., a cardiac cell. Kv4 potassium channels, e.g., potassium channels having a Kv4.2 or Kv4.3 subunit, underlie the voltage-gated K⁺ current known as Iₜₒ (transient outward current) in the mammalian heart (Kaab S. et al (1998) Circulation 98(14):1383-93; Dixon J. E. et al. (1996) Circulation Research 79(4):659-68; Nerbonne J M (1998) Journal of Neurobiology 37(1):37-59; Barry D. M. et al. (1998) Circulation Research 83(5):560-7; Barry D. M. et al. (1996) Annual Review of Physiology 58:363-94. This current underlies the rapid repolarization of cardiac myocytes during an action potential. It also participates in the inter-beat interval by controlling the rate at which cardiac myocytes reach the threshold for firing a subsequent action potential.

This current is also known to be down regulated in patients with cardiac hypertrophy, resulting in prolongation of the cardiac action potential. In these patients, action potential prolongation is thought to produce changes in calcium load and calcium handling within the myocardium, which contributes to the progression of cardiac disease from hypertrophy to heart failure (Wickenden et al. (1998) Cardiovascular Research 37:312). Interestingly, sever PCIPs of the present invention (e.g., 9ql, 9qm, 9qs, shown in SEQ ID NOs:13, 15, 17, 19, 21, 23, and 25) bind to and modulate potassium channels containing a Kv4.2 or Kv4.3 subunit and contain calcium binding EF-hand domains. Because mutations in these PCIP genes, defects in the expression of these calcium-binding PCIP proteins themselves, or defects in the interaction between these PCIPs and Kv4.2 or Kv4.3 channels, might be expected to lead to decreases in KV4.3 or Kv4.3(Iₘ) currents in the myocardium, therapeutic agents that alter PCIP expression or modulate the interaction between these PCIPs and Kv4.2 or Kv4.3 may be extremely valuable agents to slow or prevent the progression of disease from hypertrophy to heart failure.

Accordingly, in one aspect, this invention provides a method for identifying a compound suitable for treating a cardiovascular disorder by contacting a PCIP polypeptide, or a cell expressing a PCIP polypeptide with a test compound and determining whether the PCIP polypeptide binds to the test compound, thereby identifying a compound suitable for treating a potassium channel associated disorder such as a cardiovascular disorder. As used herein, a “potassium channel associated disorder” includes a disorder, disease or condition which is characterized by a misregulation of a potassium channel mediated activity. Potassium channel associated disorders can, for example, detrimentally affect the generation and distribution of electrical impulses that stimulate the cardiac muscle fibers to contract. Examples of potassium channel associated disorders include cardiovascular disorders such as arteriosclerosis, ischemia reperfusion injury, restenosis, arterial inflammation, vascular wall remodeling, ventricular remodeling, rapid ventricular pacing, coronary microembolism, tachycardia, bradycardia, pressure overload, aortic bending, coronary artery ligation, vascular heart disease, atrial fibrilation, long-QT syndrome, congestive heart failure, sinus node disfunction, angina, heart failure, hypertension, atrial fibrillation, atrial flutter, dilated cardiomyopathy, idiopathic cardiomyopathy, myocardial infarction, coronary artery disease, coronary artery spasm, or arrhythmia. In a preferred embodiment, the cardiovascular disorder is associated with an abnormal Iₜₒ current.

In a preferred embodiment, the binding of the test compound to the PCIP polypeptide is detected by direct detection of test compound/polypeptide binding. In another embodiment, the binding of the test compound to the PCIP polypeptide is detected by using a competition binding assay. In yet another embodiment, the binding of the test compound to the PCIP polypeptide is detected by using an assay for PCIP activity. As used interchangeably herein, a “PCIP activity”, “biological activity of PCIP” or “functional activity of PCIP”, refers to an activity exerted by a PCIP protein, polypeptide or nucleic acid molecule on a PCIP responsive cell or on a PCIP protein substrate, as determined in vivo, or in vitro, according to standard techniques. In one embodiment, a PCIP activity is a direct activity, such as an association with a PCIP-target molecule. As used herein, a “target molecule” or “binding partner” is a molecule with which a PCIP protein binds or interacts in nature, such that PCIP-mediated function is achieved. A PCIP target molecule can be a non-PCIP molecule or a PCIP protein or polypeptide. In an exemplary embodiment, a PCIP target molecule is a PCIP ligand. Alternatively, a PCIP activity is an indirect activity, such as a cellular signaling activity mediated by interaction of the PCIP protein with a PCIP ligand.

The biological activities of PCIP are described herein. For example, the binding of the test compound to the PCIP polypeptide is detected by using an assay for one or more of the following activities: (1) interaction with (e.g., binding to) a potassium channel protein or portion thereof, e.g., a potassium channel comprising a Kv4.3 or Kv4.2 subunit; (2) regulation of the phosphorylation state of a potassium channel protein or portion thereof, (3) association with (e.g., binding to) calcium and acting as a calcium dependent kinase; (4) modulation of a potassium channel mediated activity in a cell (e.g., a cardiac cell such as a pericardial cell, a myocardial cell, or an endocardial cell); (5) modulation of chromatin formation in a cell, e.g., a cardiac cell; (6) modulation of vesicular traffic and protein transport in a cell, e.g., a cardiac cell; (7) modulation of cytokine signaling in a cell, e.g., a cardiac cell; (8) regulation of the association of a potassium channel protein or portion thereof with the cellular cytoskeleton; (9) modulation of cellular proliferation; (10) modulation of the release of neurotransmitters; (11) modulation of membrane excitability; (12) influencing the resting potential of membranes; (13) modulation of wave forms and frequencies of action potentials; and (14) modulation of thresholds of excitation.

In another aspect, the invention features a method for identifying a compound suitable for treating a cardiovascular disorder by incubating a cell expressing a potassium channel or a fragment thereof, and a PCIP polypeptide, in the presence and absence of a candidate compound; and determining whether the presence of the candidate compound modulates the interaction of the potassium channel or fragment thereof with the PCIP polypeptide, thereby identifying a compound suitable for treating a cardiovascular disorder. As used herein, a “potassium channel” includes a protein or polypeptide that is involved in receiving, conducting, and transmitting signals in an excitable cell. Potassium channels are typically expressed in electrically excitable cells, e.g., neurons, cardiac, skeletal and smooth muscle, renal, endocrine, and egg cells, and can form heteromultimeric structures, e.g., composed of pore-forming and cytoplasmic subunits. Examples of potassium channels include: (1) the voltage-gated potassium channels, (2) the ligand-gated potassium channels, and (3) the mechanically-gated potassium channels. For a detailed description of potassium channels, see Kandel E. R. et al., Principles of Neural Science, second edition, (Elsevier Science Publishing Co., Inc., N.Y. (1985)), the contents of which are incorporated herein by reference. The PCIP proteins of the present invention have been shown to interact with, for example, potassium channels having a Kv4.3 subunit or a Kv4.2 subunit.

In yet another aspect, the invention features a method for treating a cardiovascular disorder by contacting a potassium channel with an effective amount of a compound that modulates the binding of a PCIP protein to the potassium channel.

In a further aspect, the invention features a method for determining if a subject is at risk for a cardiovascular disorder by detecting, in a sample of cells from the subject an alteration in a PCIP gene which causes a mutated PCIP polypeptide to be produced, an alteration in a PCIP gene which causes abnormal expression of a PCIP polypeptide, or an alteration in a PCIP gene which causes abnormal processing of a PCIP polypeptide.

In another aspect, the invention features a method for identifying a subject suffering from a cardiovascular disorder by detecting, in a sample of cells from the subject an alteration in a PCIP gene which causes a mutated PCIP polypeptide to be produced, an alteration in a PCIP gene which causes abnormal expression of a PCIP polypeptide, or an alteration in a PCIP gene which causes abnormal processing of a PCIP polypeptide.

The PCIP molecules of the present invention were initially identified based on their ability, as determined using yeast two-hybrid assays (described in detail in Example 1), to interact with the amino-terminal 180 amino acids of rat Kv4.3 subunit. Further binding studies with other potassium subunits were performed to demonstrate specificity of the PCIP for Kv4.3 and Kv4.2. In situ localization, immuno-histochemical methods, co-immunoprecipitation and patch clamping methods were then used to clearly demonstrate that the PCIPs of the present invention interact with and modulate the activity of potassium channels, particularly those comprising a 4.3 or 4.2 subunit.

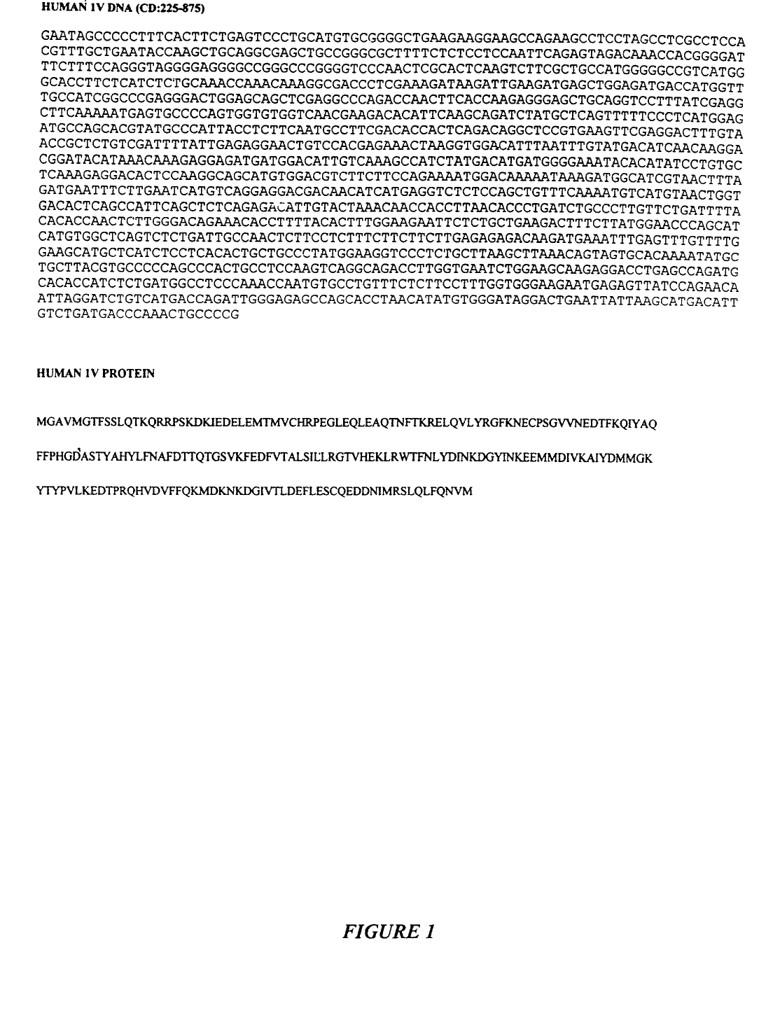

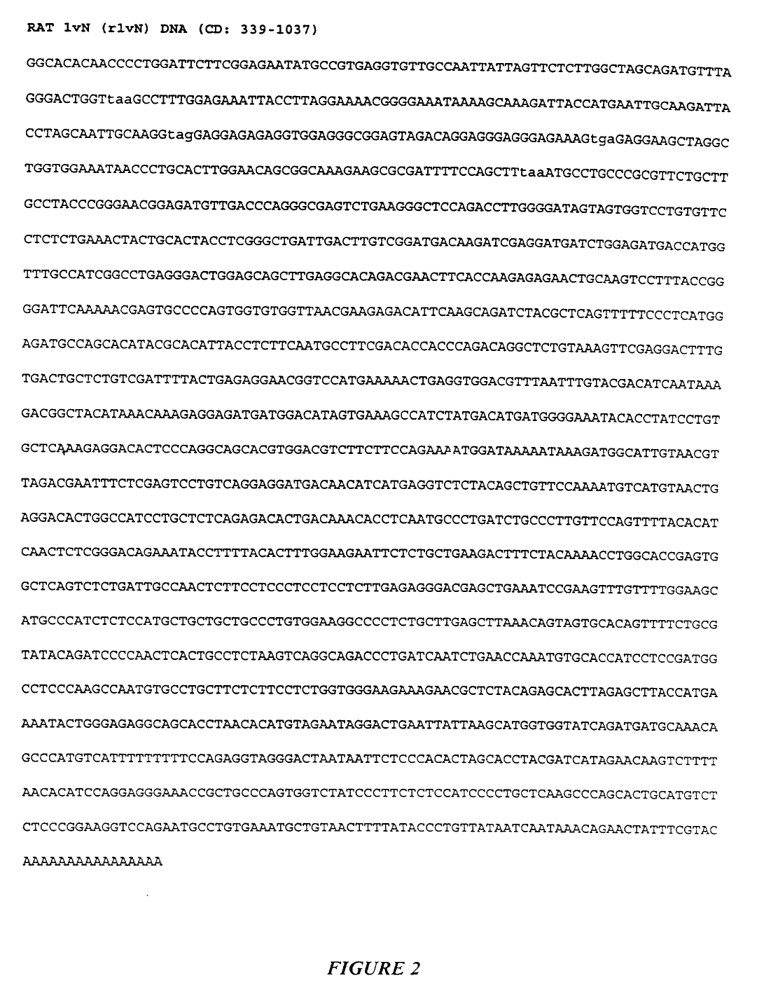

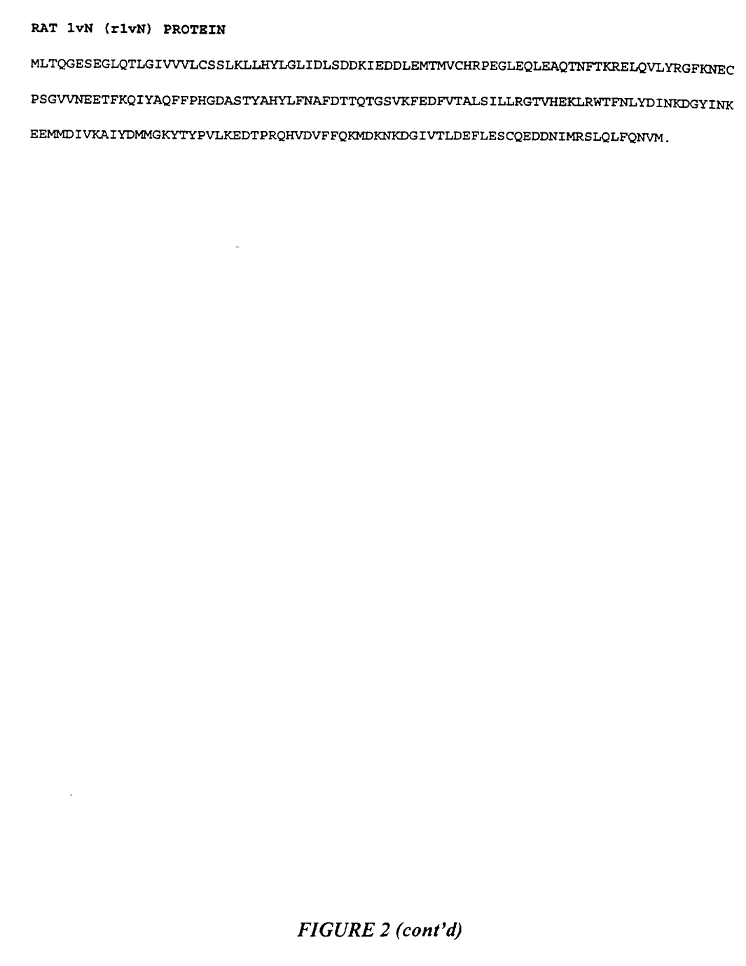

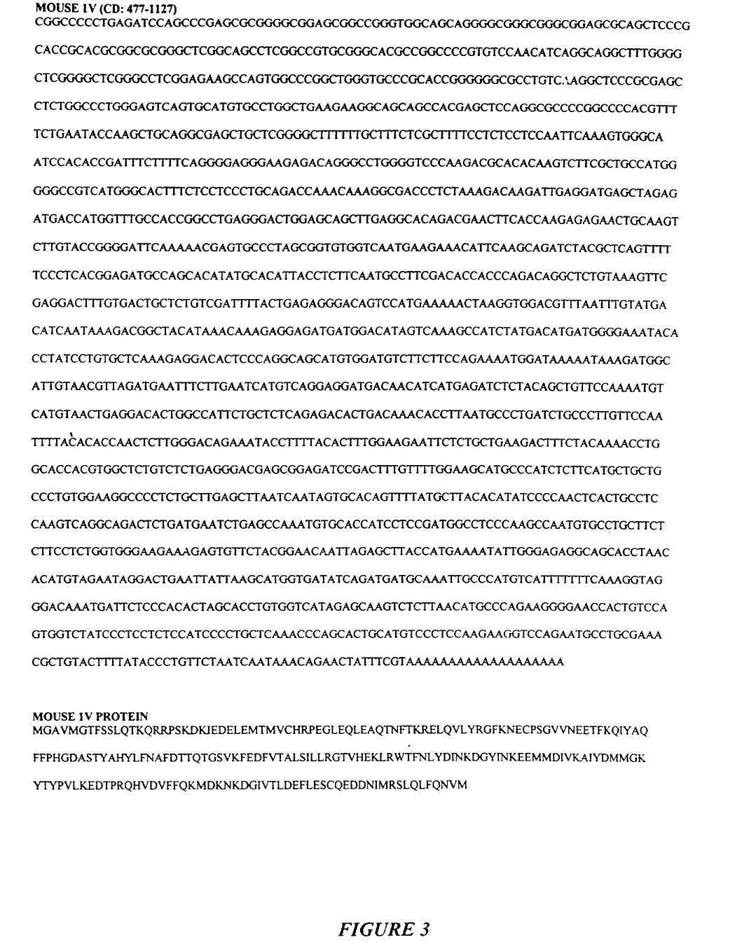

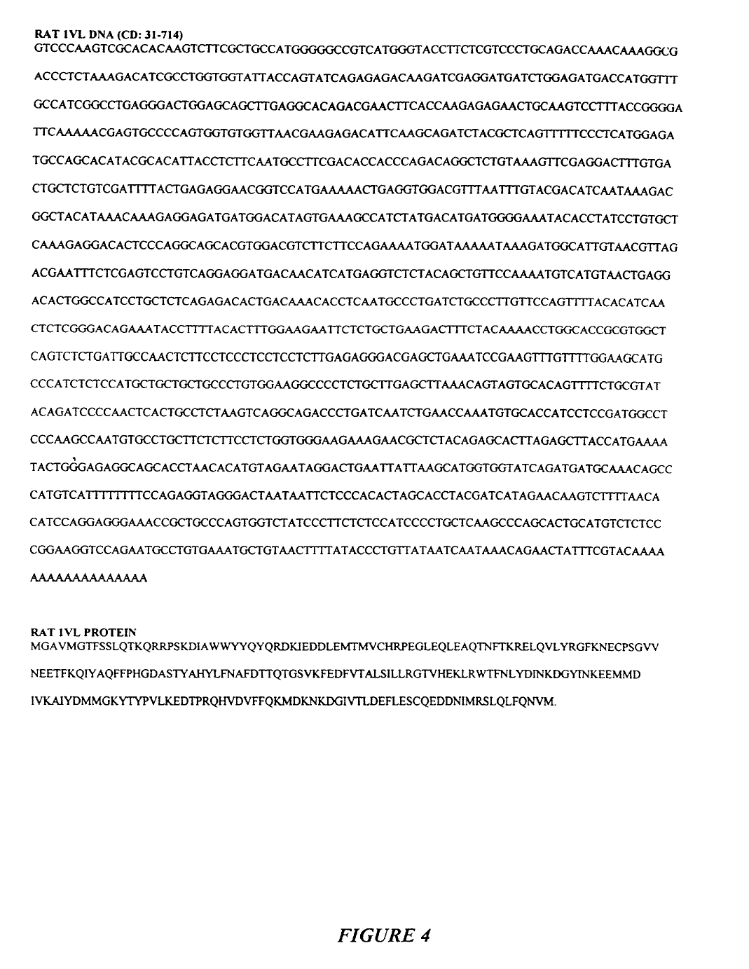

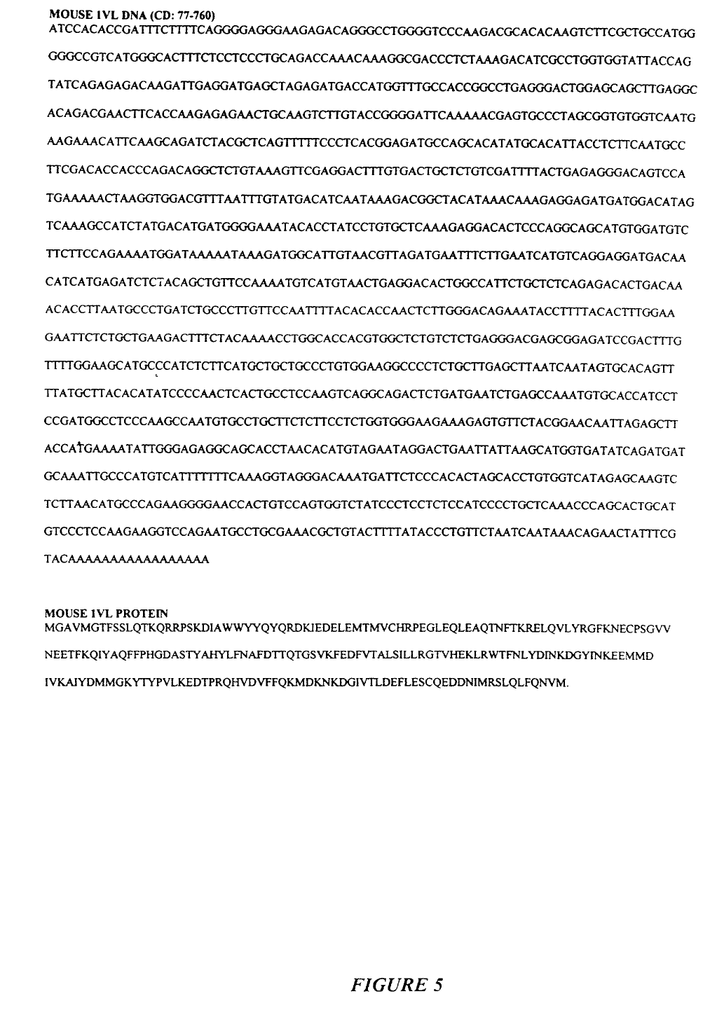

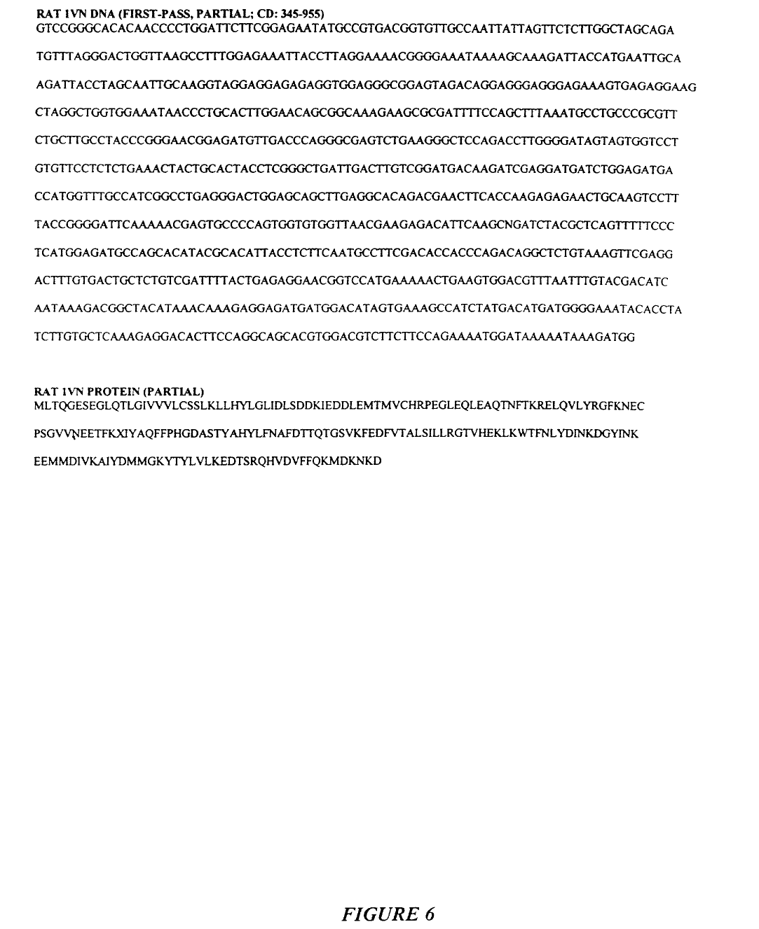

Several novel human, mouse, monkey, and rat PCIP family members have been identified, referred to herein as 1v, 9q, p19, W28559, KChIP4, 33b07, 1p, and rat 7s proteins and nucleic acid molecules. The human, rat, and mouse cDNAs encoding the 1v polypeptide are represented by SEQ ID NOs:1, 3, and 5, and shown in FIGS. 1, 2, and 3, respectively. In the brain, 1v mRNA is highly expressed in neocortical and hippocampal interneurons, in the thalamic reticular nucleus and medial habenula, in basal forebrain and striatal cholinergic neurons, in the superior colliculus, and in cerebellar granule cells. The 1v polypeptide is highly expressed in the somata, dendrites, axons and axon terminals of cells that express 1v mRNA. Splice variants of the 1v gene have been identified in rat and mouse and are represented by SEQ ID NOs:7, 9, and 11 and shown in FIGS. 4, 5, and 6, respectively. 1v polypeptide interacts with potassium channels comprising Kv4.3 or Kv4.2 subunits, but not with Kv1.1 subunits. As determined by Northern blot, the 1v transcripts (mRNA) are expressed predominantly in the brain.

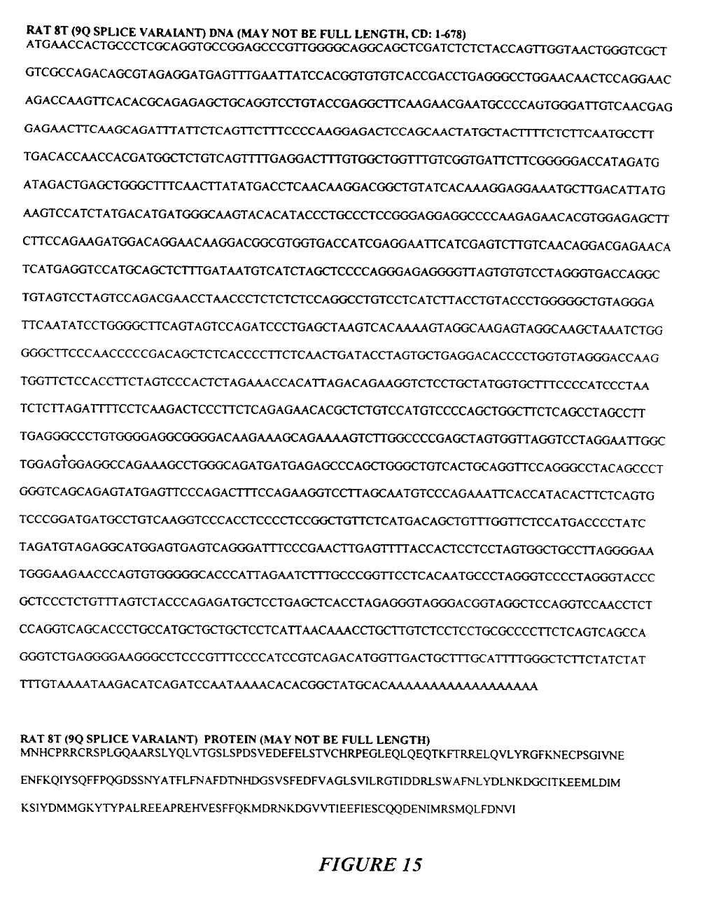

The 8t cDNA (SEQ ID NO:29) encodes a polypeptide having a molecular weight of approximately 26 kD corresponding to SEQ ID NO:30 (see FIG. 15). The 8t polypeptide interacts with potassium channel comprising Kv4.3 or Kv4.2 subunits, but not with Kv1.1 subunits. As determined by Northern blot and in situ data, the 8t mRNA is expressed predominantly in the heart and the brain. The 8t cDNA is a splice variant of 9q.

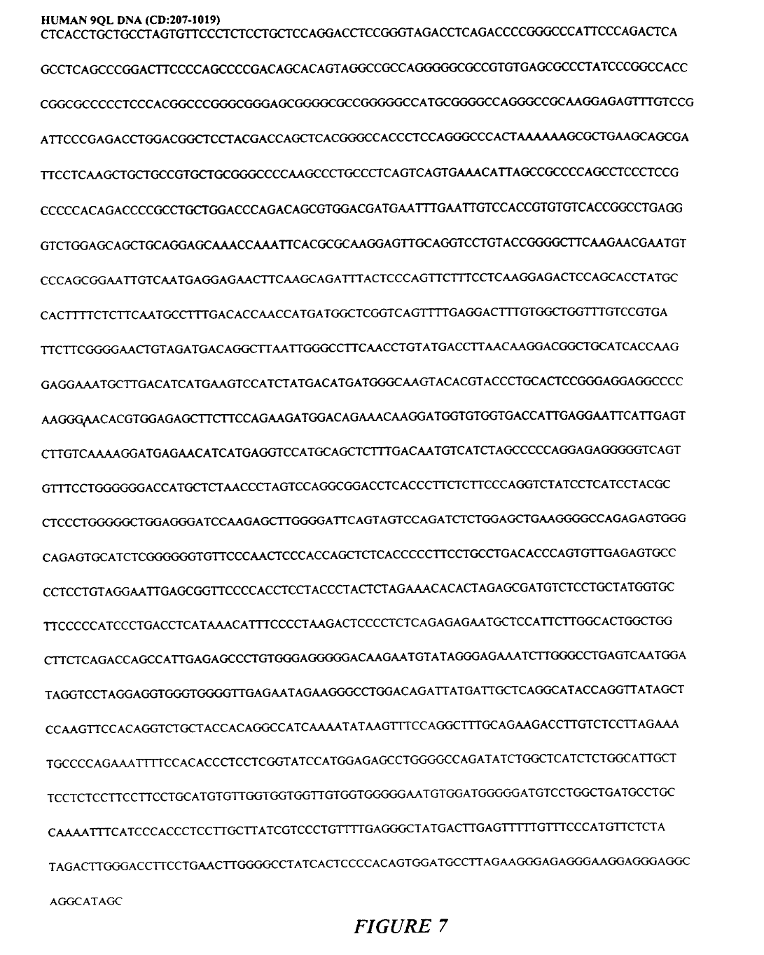

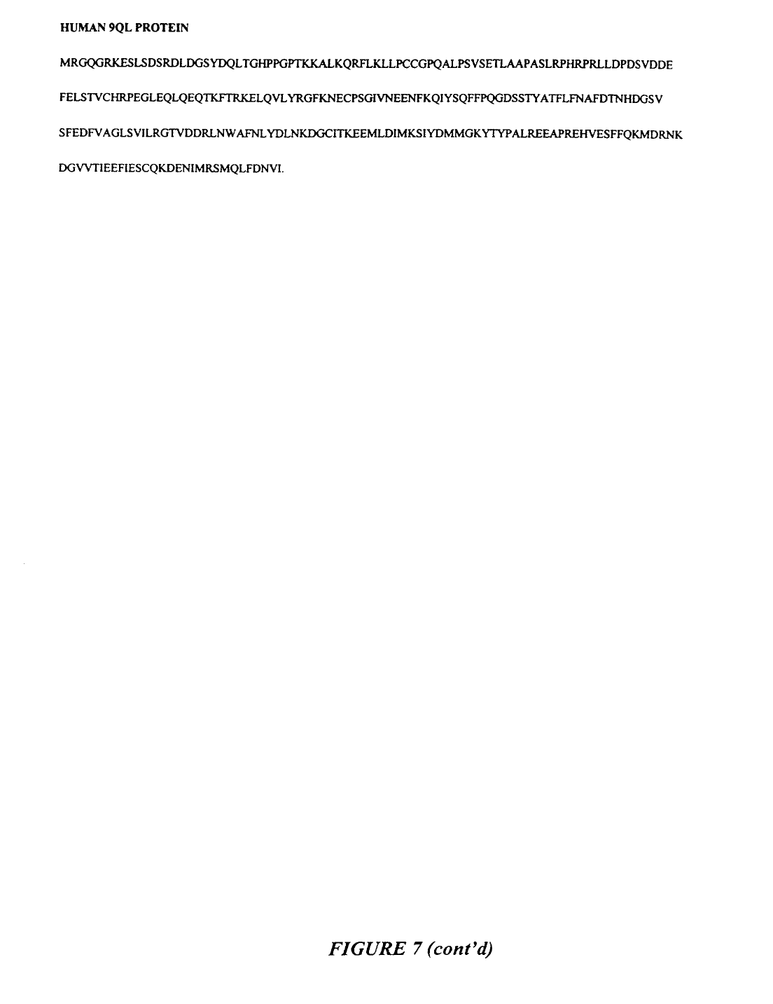

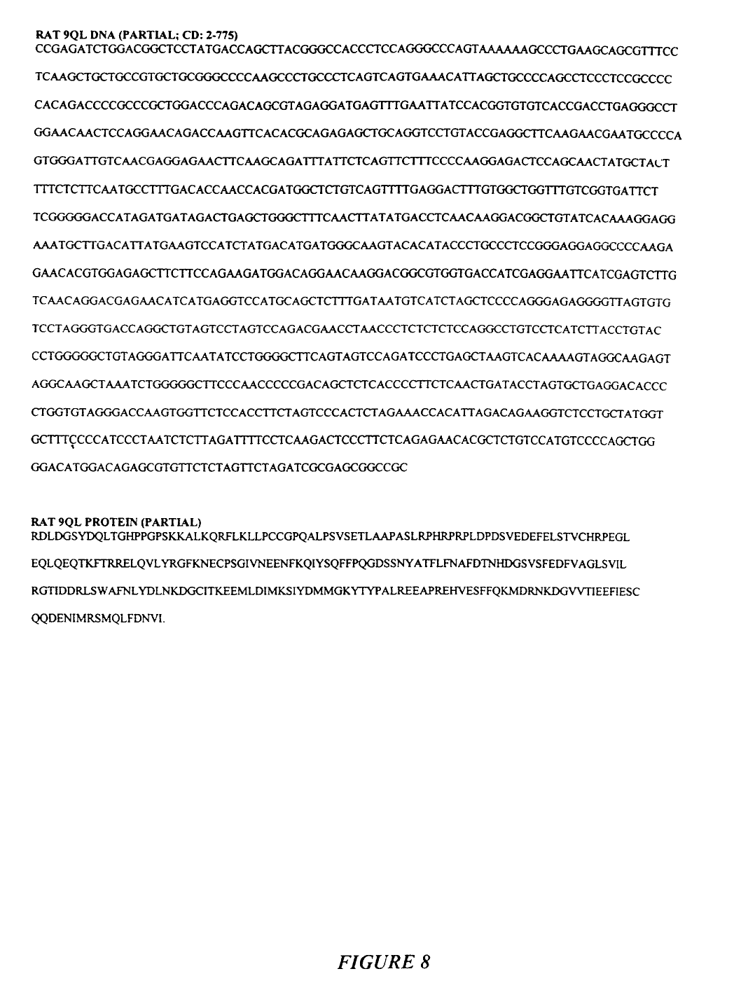

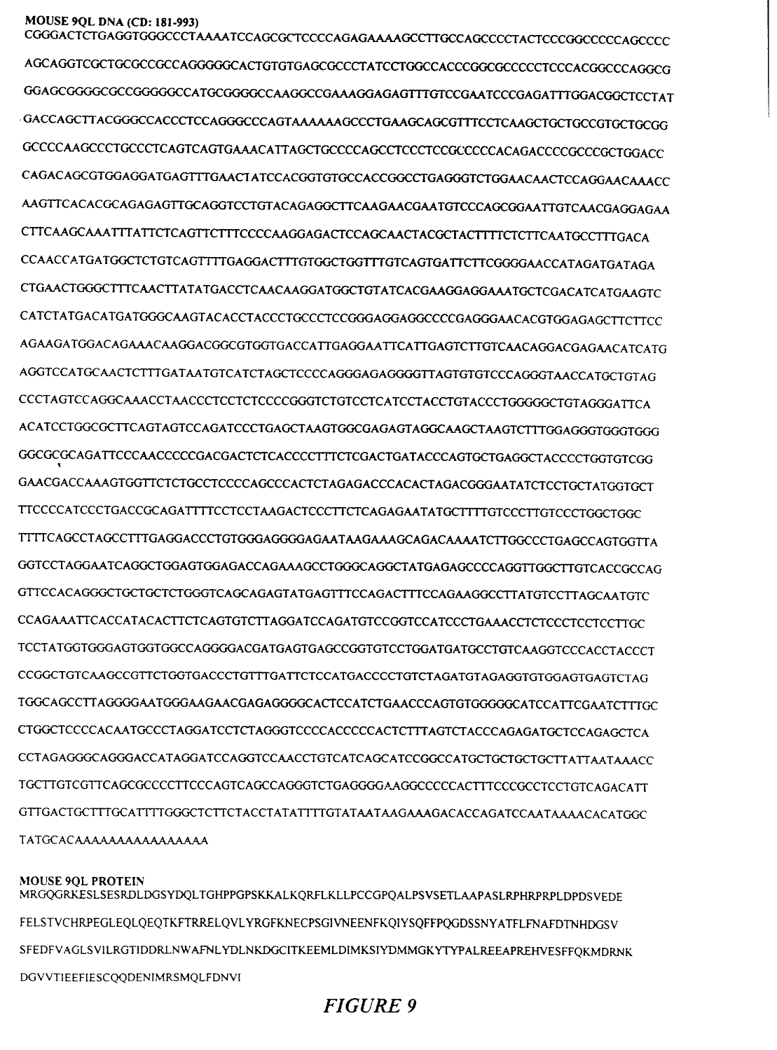

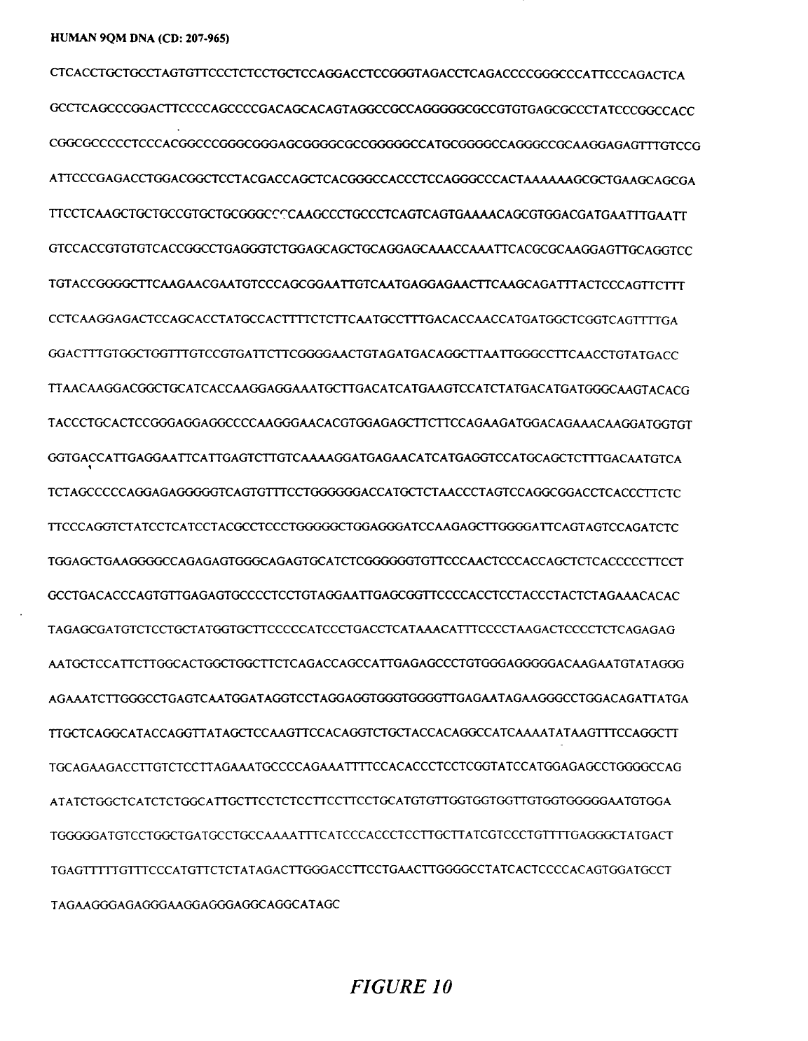

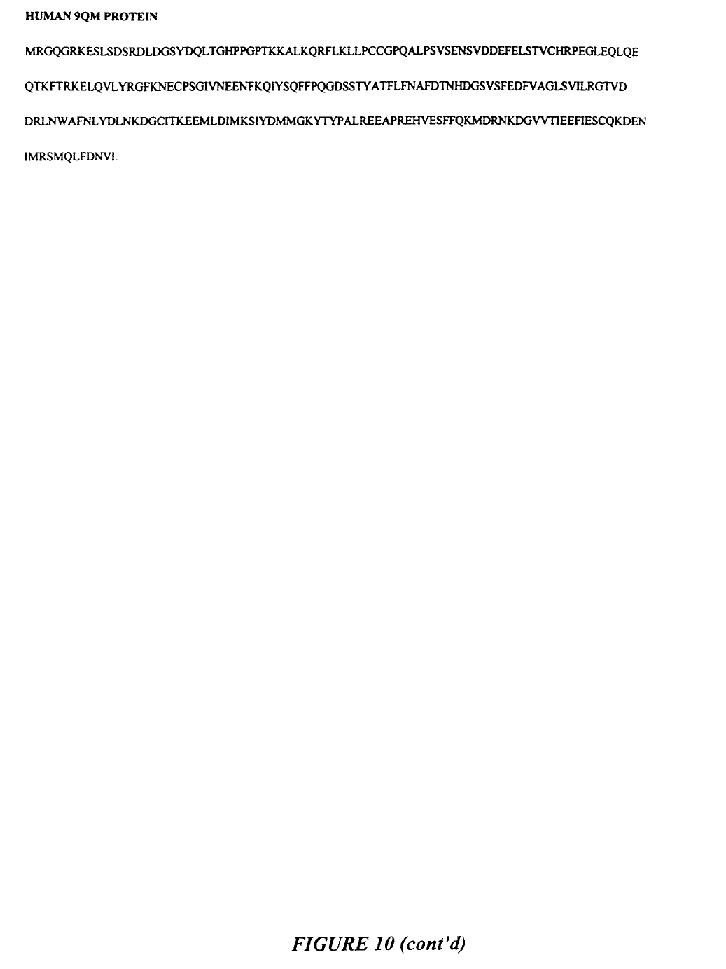

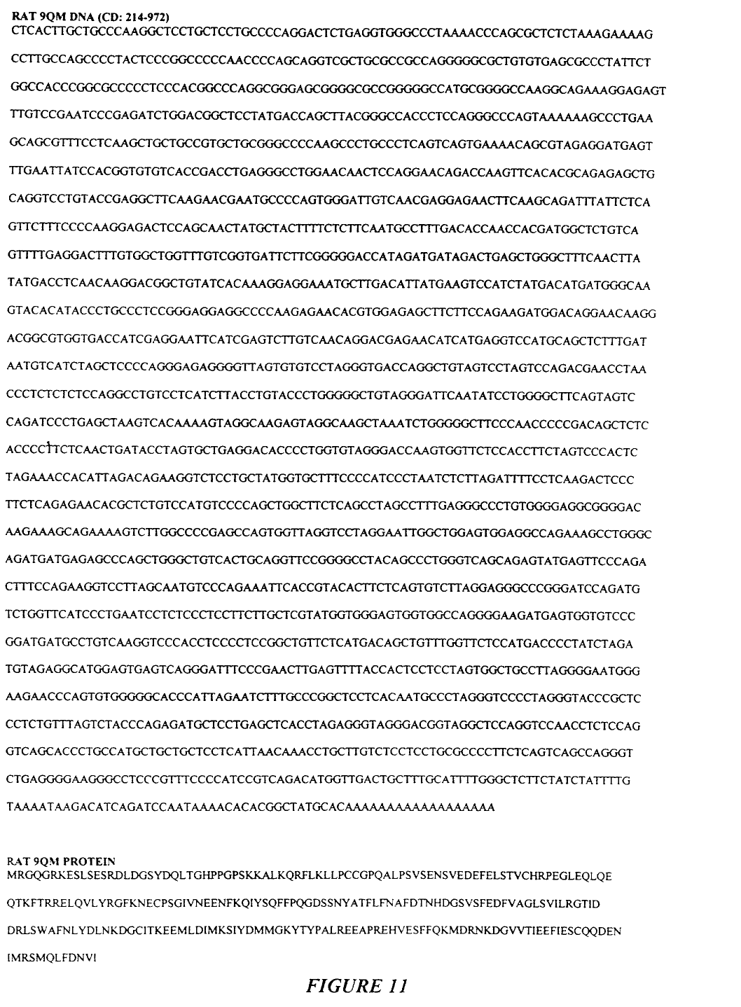

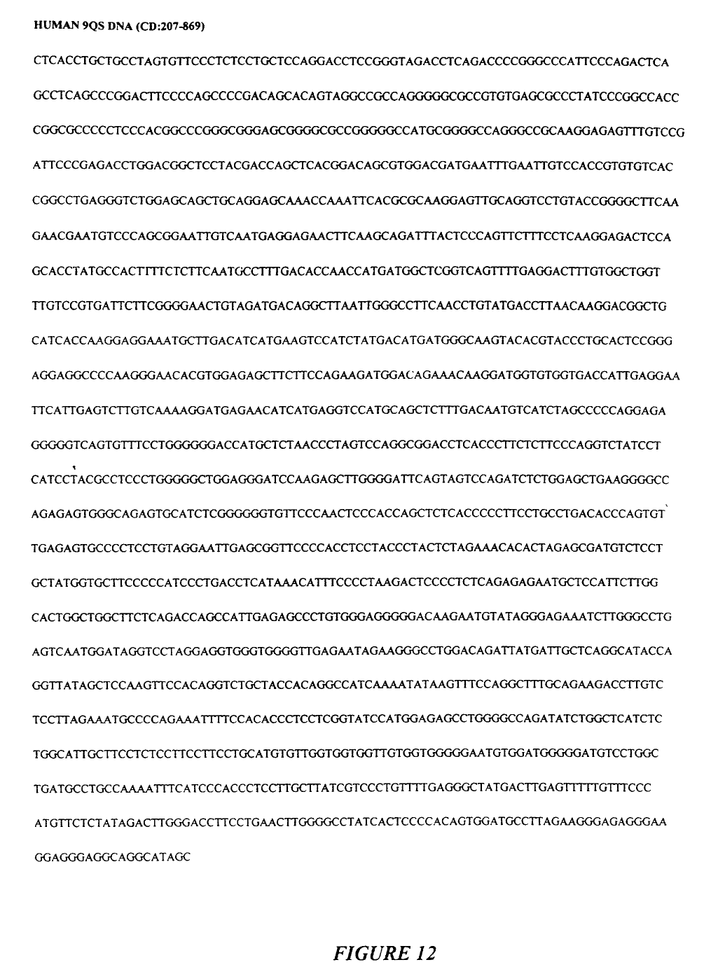

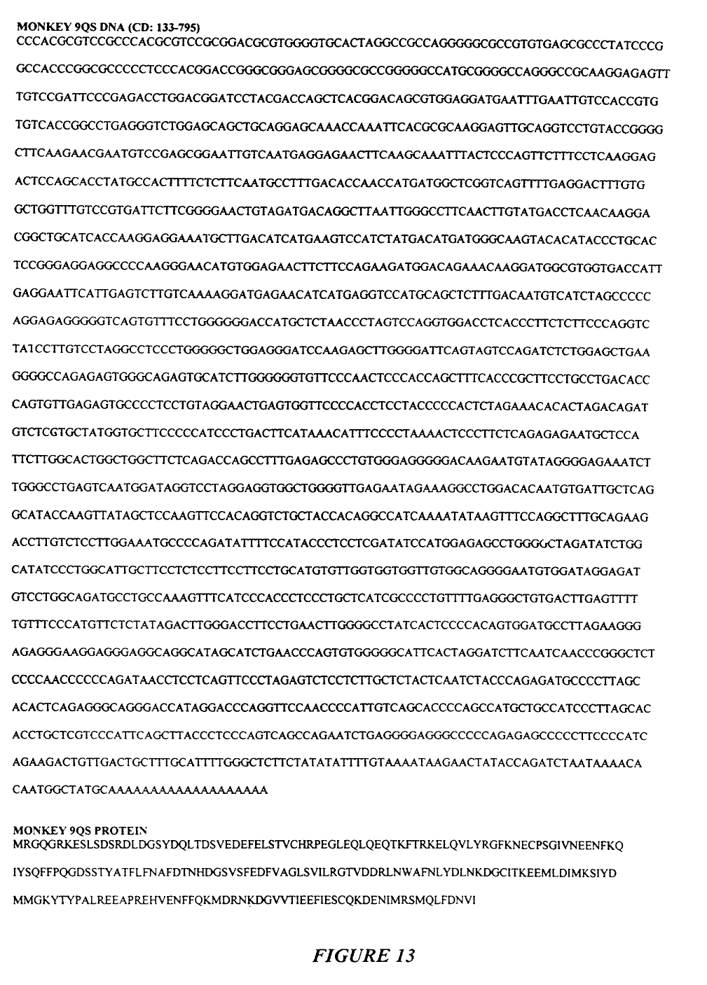

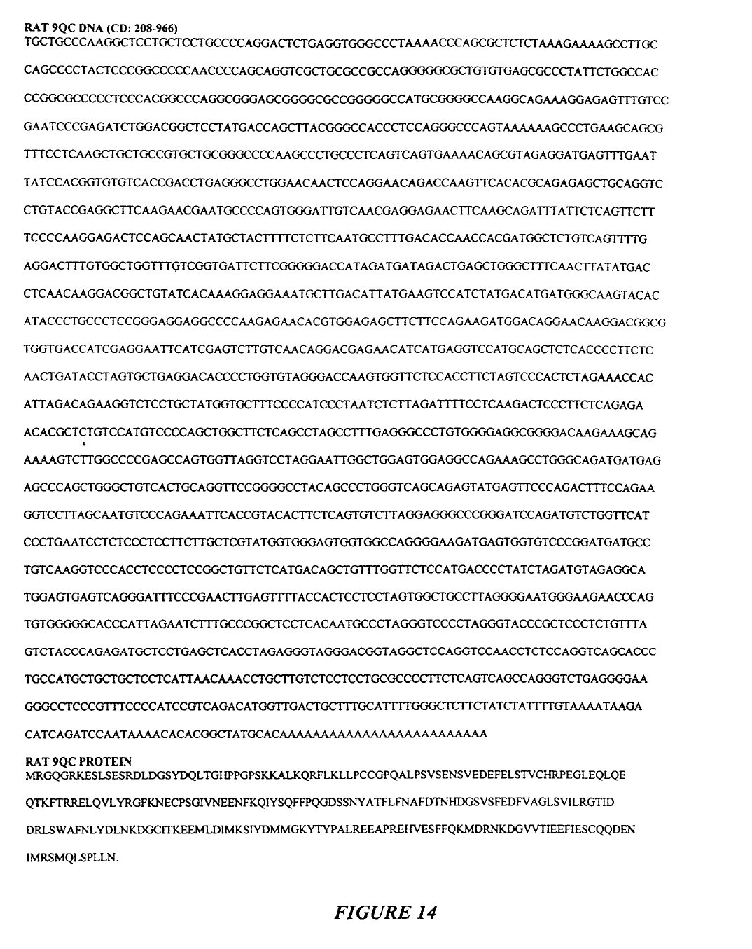

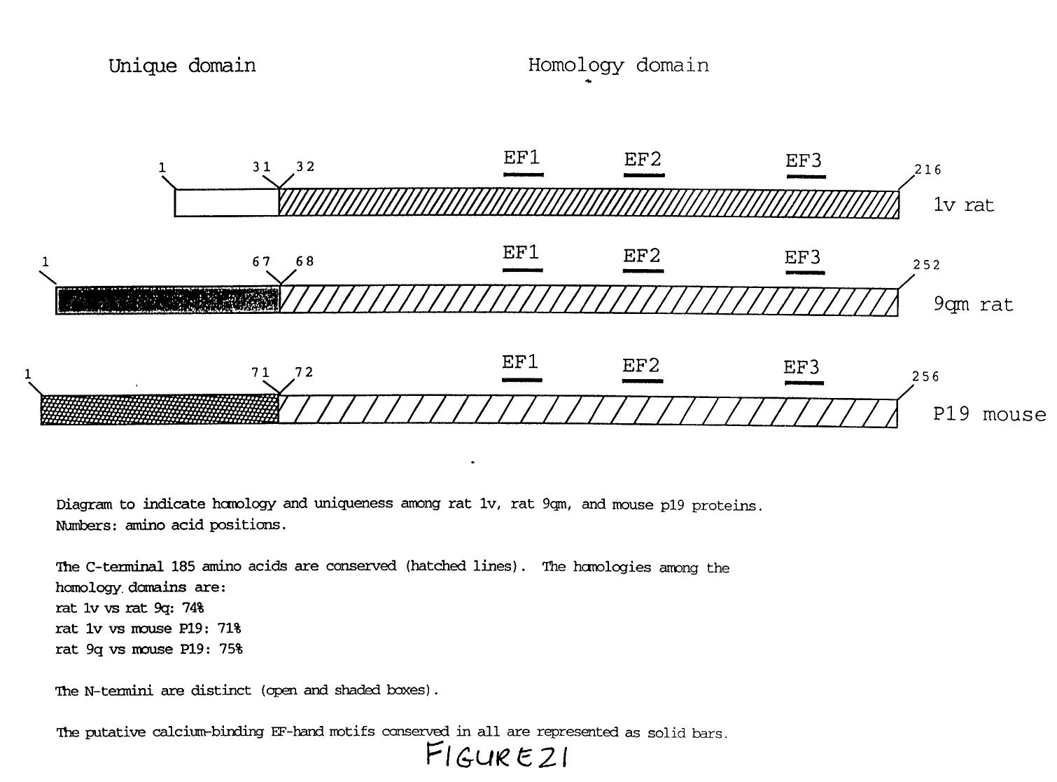

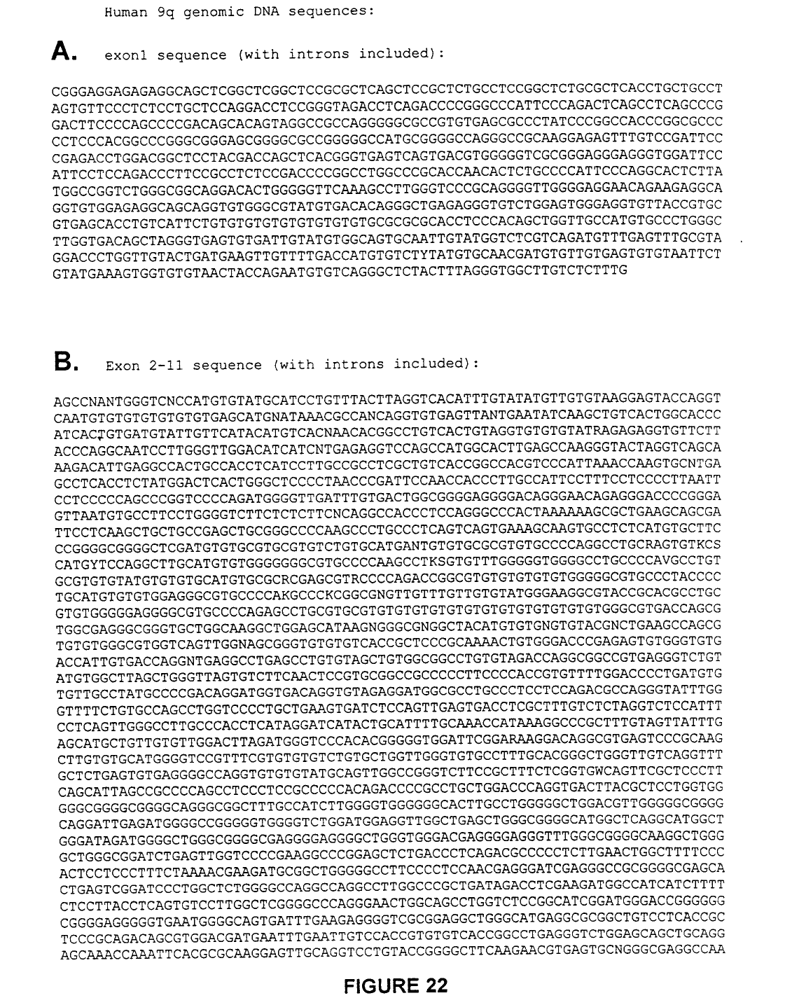

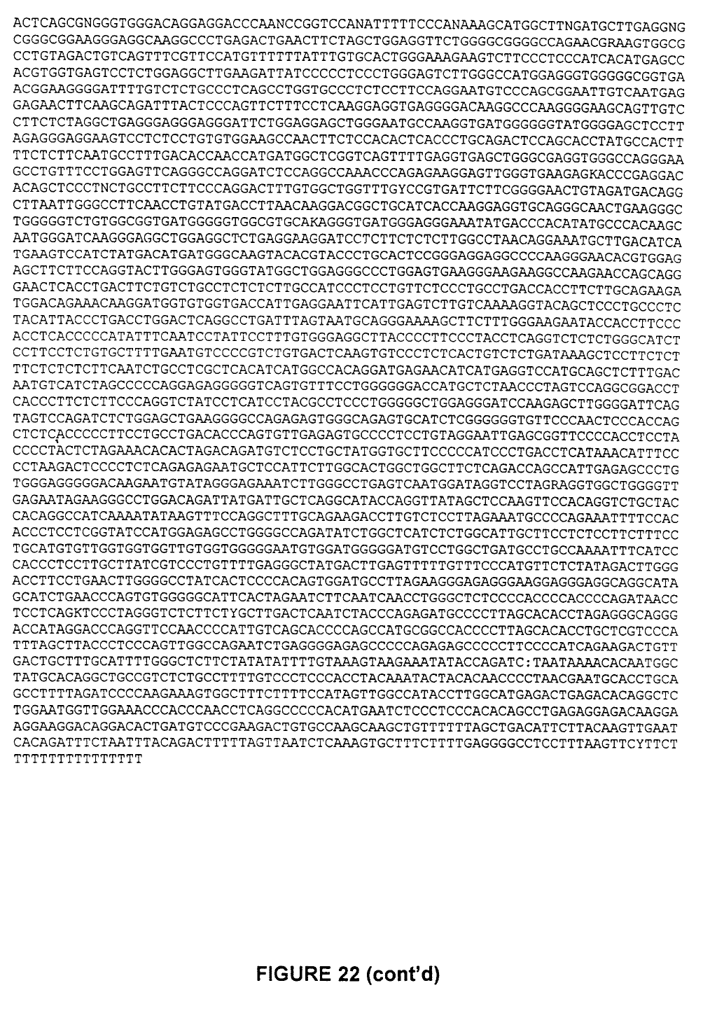

Human, rat, monkey, and mouse 9q cDNA was also isolated. Splice variants include human 9ql (SEQ ID NO:13; FIG. 7) rat 9ql (SEQ ID NO:15; FIG. 8), mouse 9ql (SEQ ID NO:17; FIG. 9), human 9qm (SEQ ID NO:19; FIG. 10), rat 9qm (SEQ ID NO:21; FIG. 11), human 9qs (SEQ ID NO:23; FIG. 12), monkey 9qs (SEQ ID NO:25; FIG. 13), and rat 9qc (SEQ ID NO:27; FIG. 14). The genomic DNA sequence of 9q has also be determined. Exon 1 and its flanking intron sequences (SEQ ID NO:46) are shown in FIG. 22A. Exons 2-11 and the flanking intron sequences (SEQ ID NO:47) are shown in FIG. 22B. 9q polypeptides interact with potassium channels comprising Kv4.3 or Kv4.2 subunits, but not with Kv1.1 subunits. As determined by Northern blot and in situ data, the 9q proteins are expressed predominantly in the heart and the brain. In the brain, 9q mRNA is highly expressed in the neostriatum, hippocampal formation, neocortical pyramidal cells and intemeurons, and in the thalamus, superior colliculus, and cerebellum.

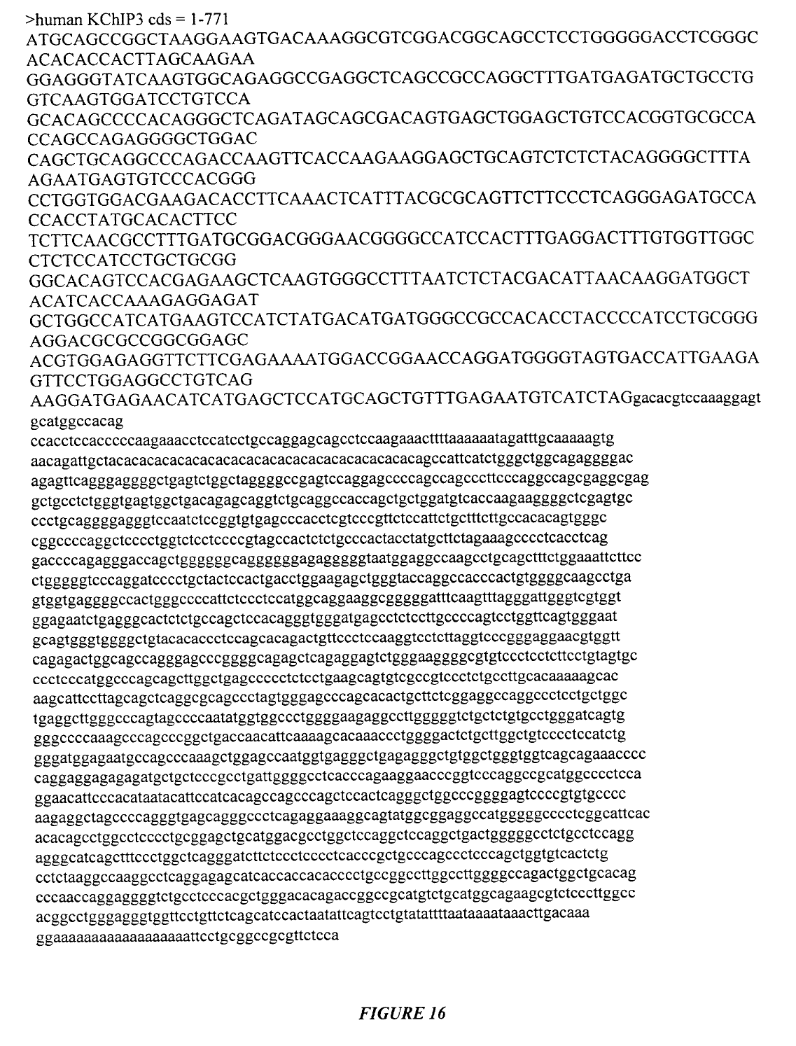

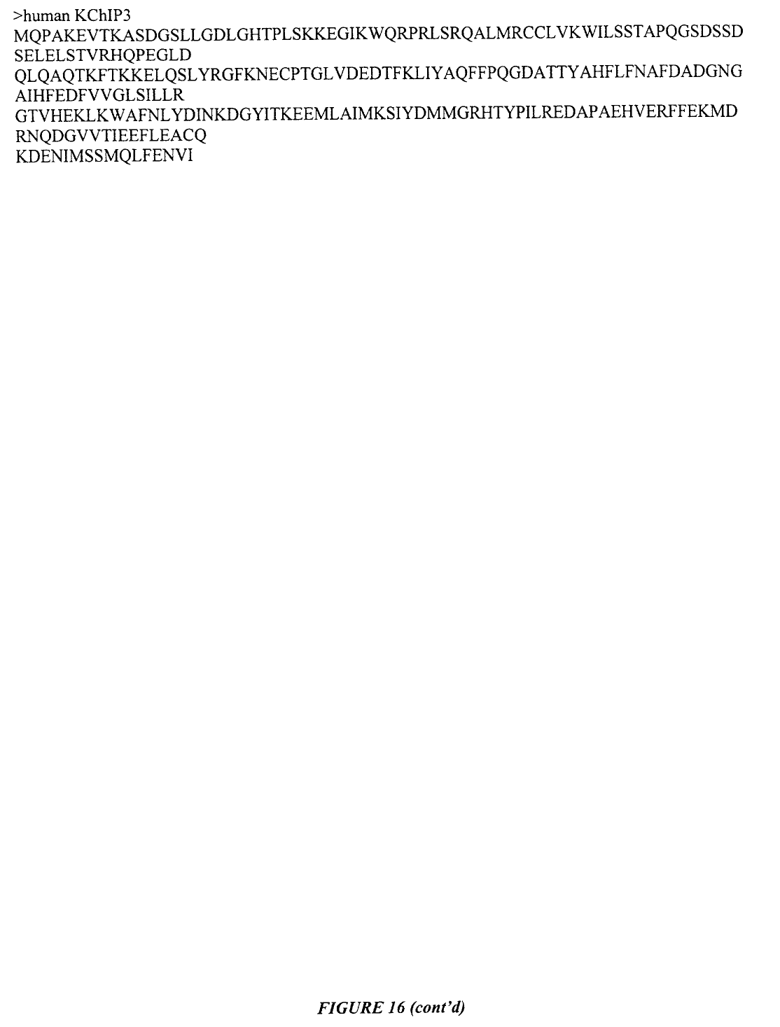

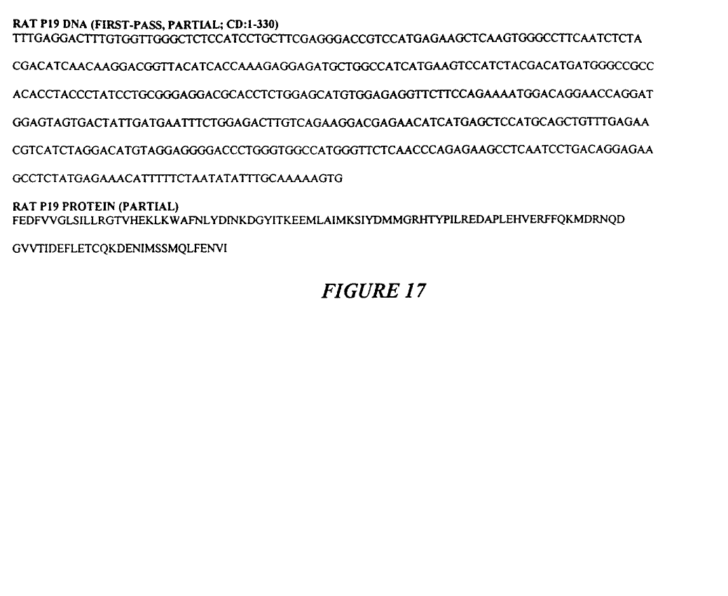

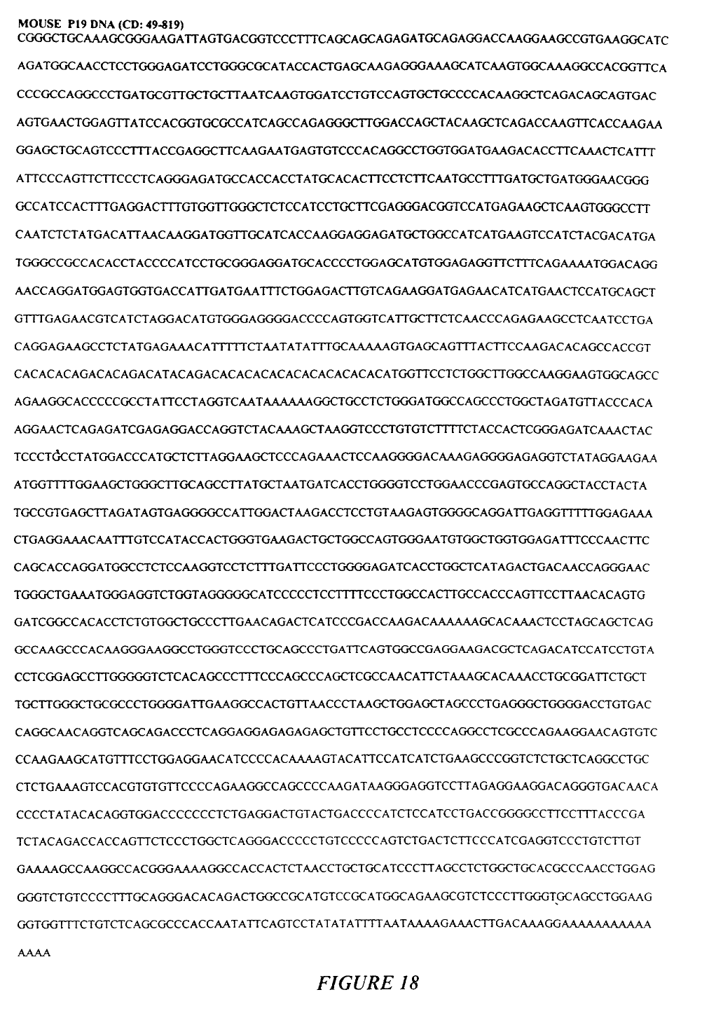

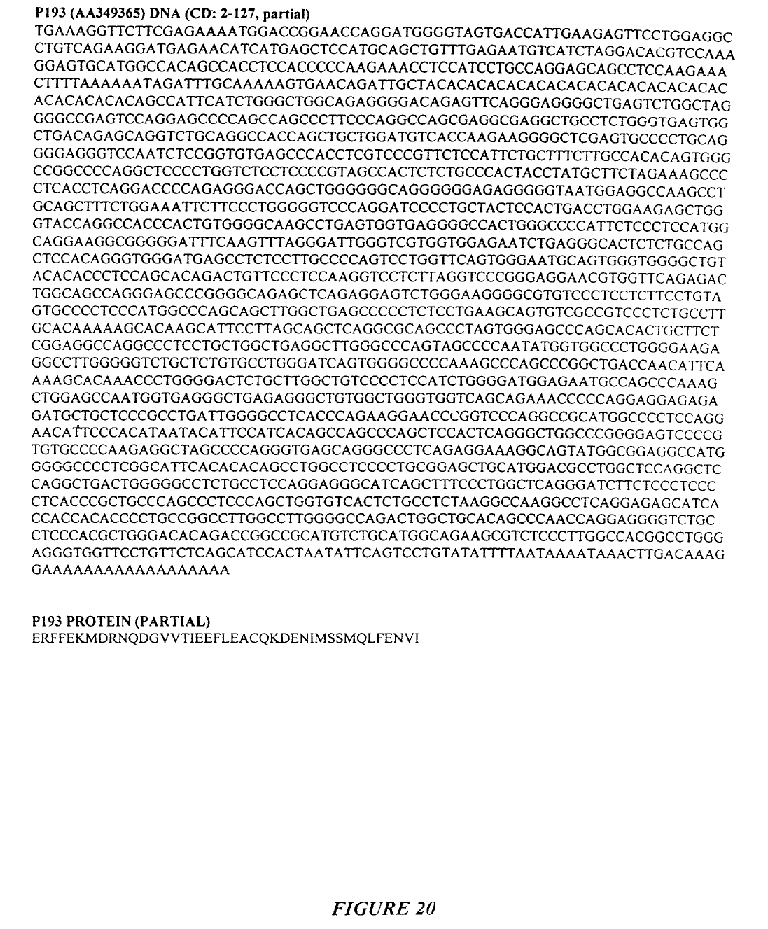

Human, rat, and mouse P19 cDNA were also isolated. Human P19 is shown in SEQ ID NO:31 and FIG. 16; and in SEQ ID NO:39 and FIG. 20 (the 3′ sequence). Rat P19 is shown in SEQ ID NO:33 and FIG. 17, and mouse P19 is shown in SEQ ID NO:35 and FIG. 18. P19 polypeptides interact with potassium channels comprising Kv4.3 or Kv4.2 subunits, but not with Kv1.1 subunits. As determined by northern blot analysis, the P19 transcripts (mRNA) are expressed predominantly in the brain and to a much lesser degree in the heart.

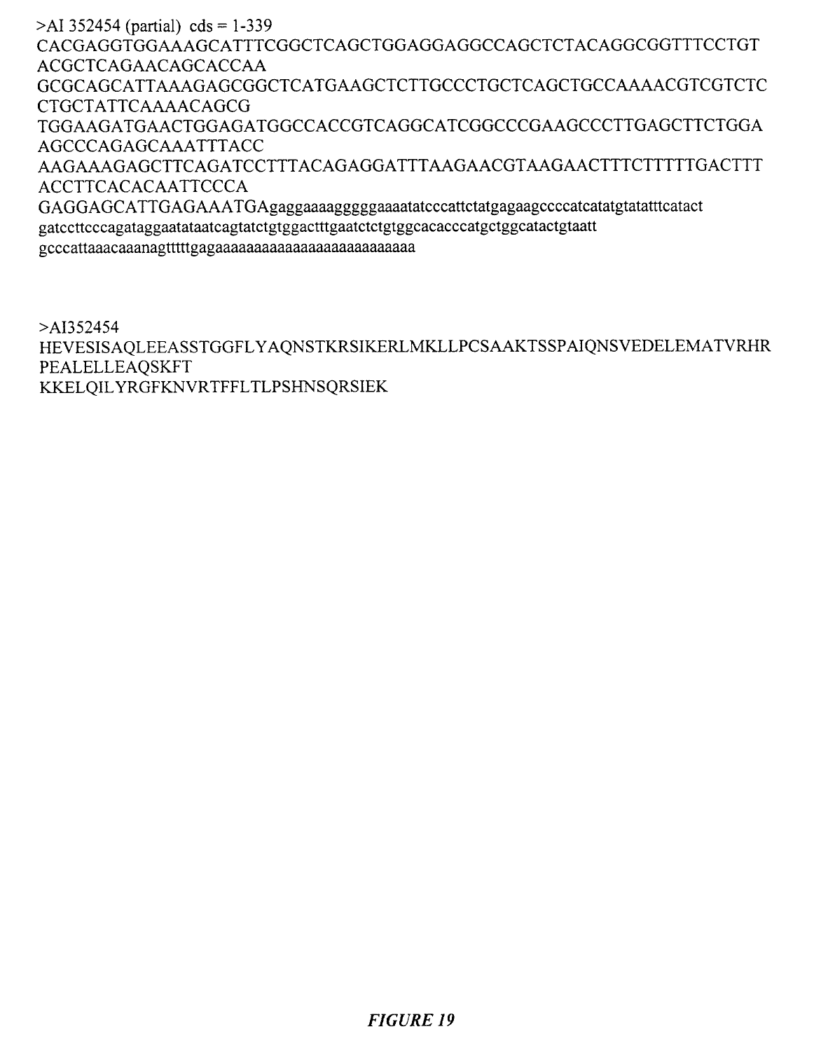

A partial human paralog of the PCIP molecules was also identified. This paralog is referred to herein as W28559 and is shown in SEQ ID NO:37 and FIG. 19.

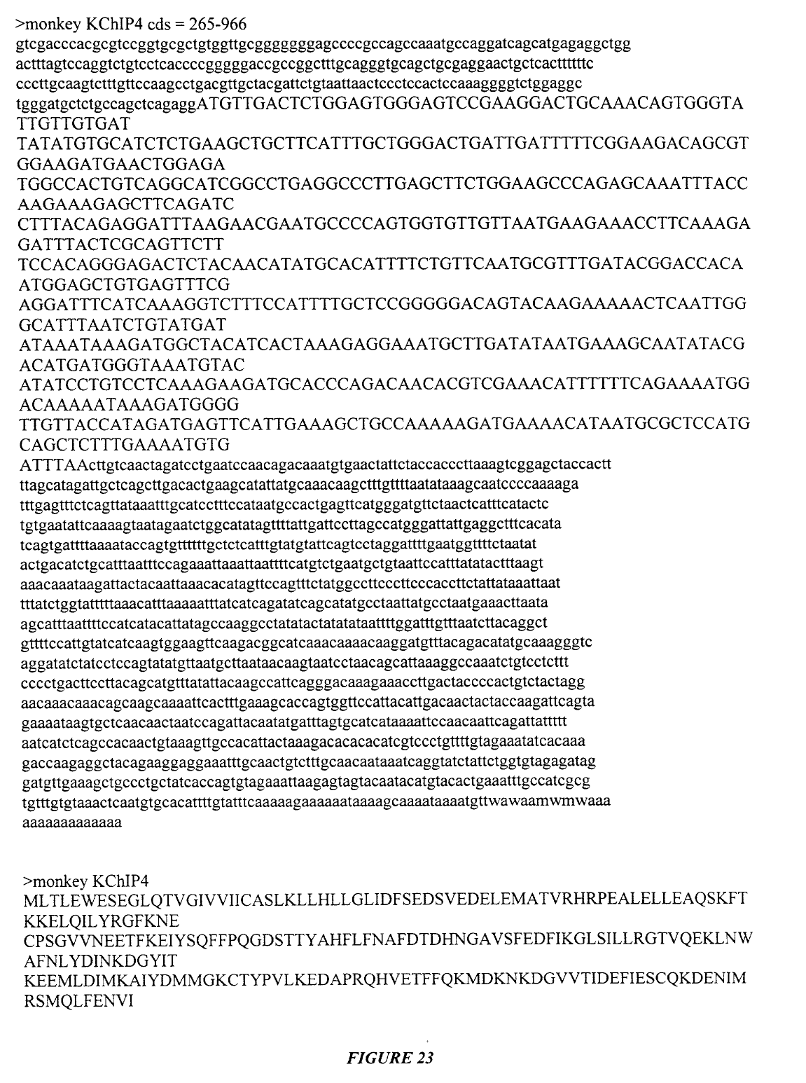

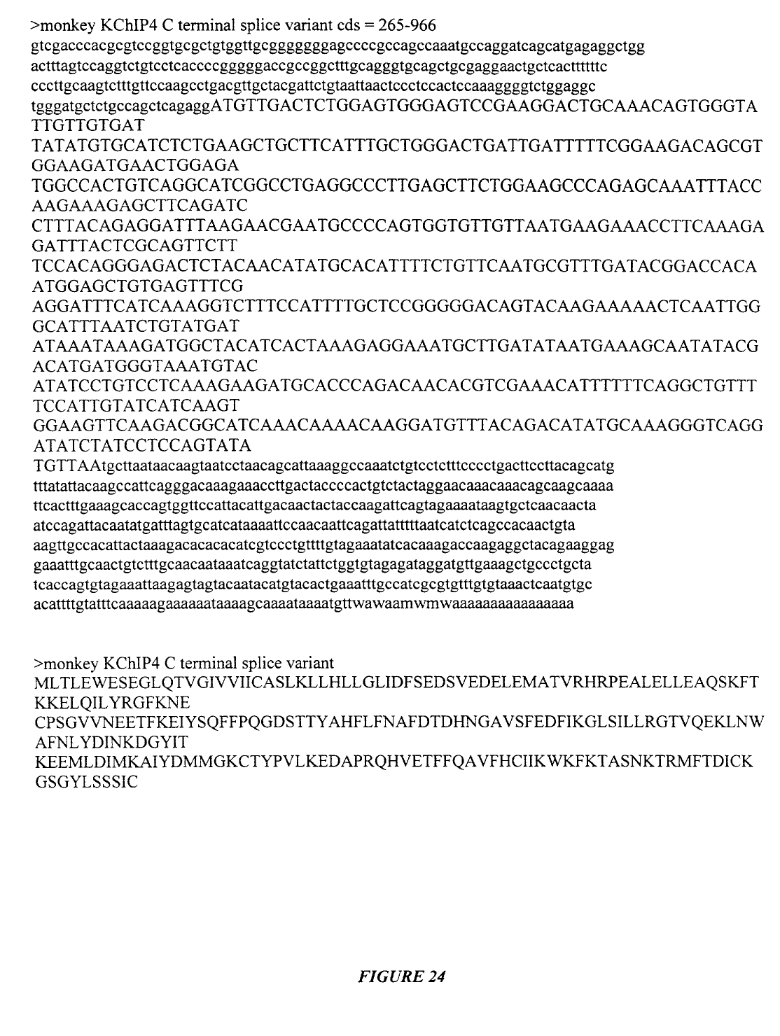

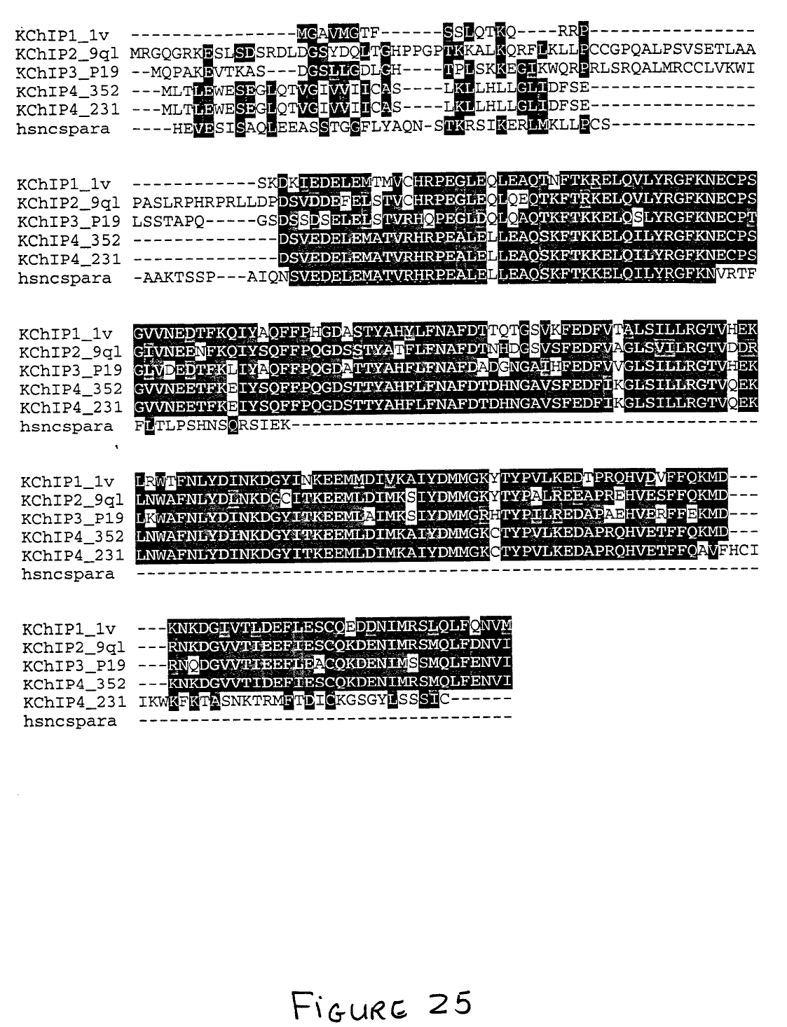

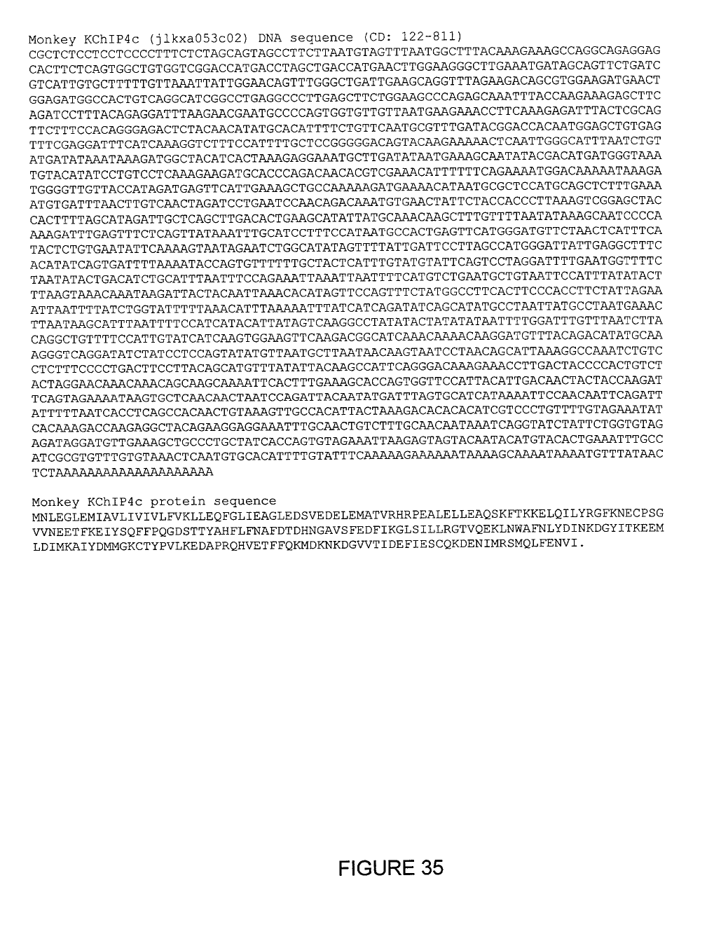

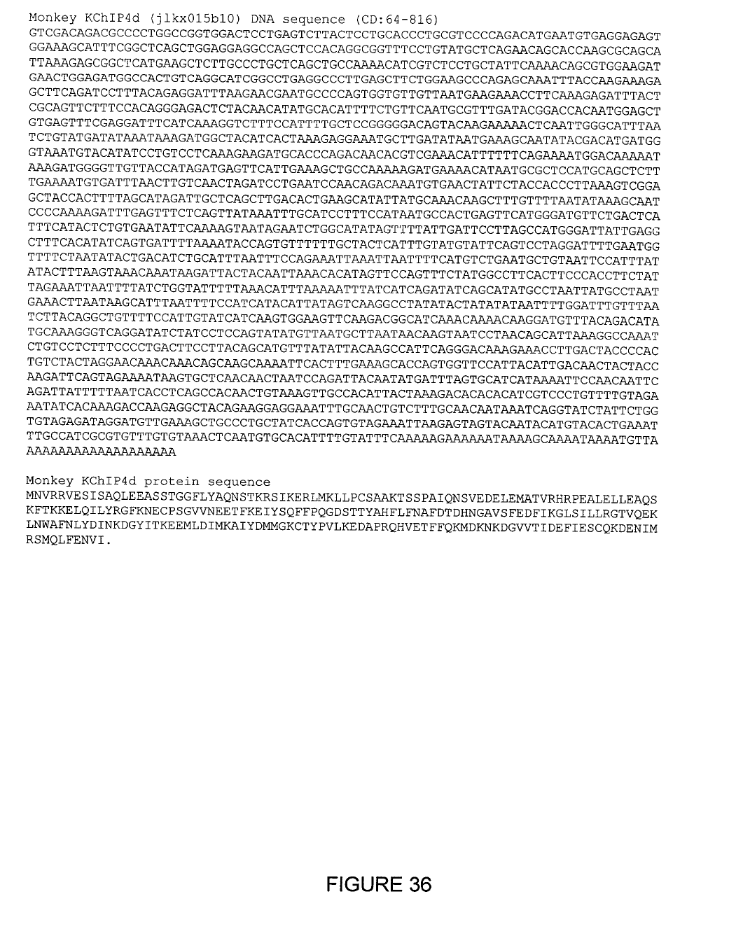

Monkey KChIP4a and its splice variants KChIP4b, KChIP4c, and KChIP4d were also identified. Monkey KChIP4a is shown in SEQ ID NO:48 and FIG. 23. Monkey KChIP4b is shown in SEQ ID NO:50 and FIG. 24. Monkey KChIP4c is shown in SEQ ID NO:69 and FIG. 35. Monkey KChIP4d is shown in SEQ ID NO:71 and FIG. 36.

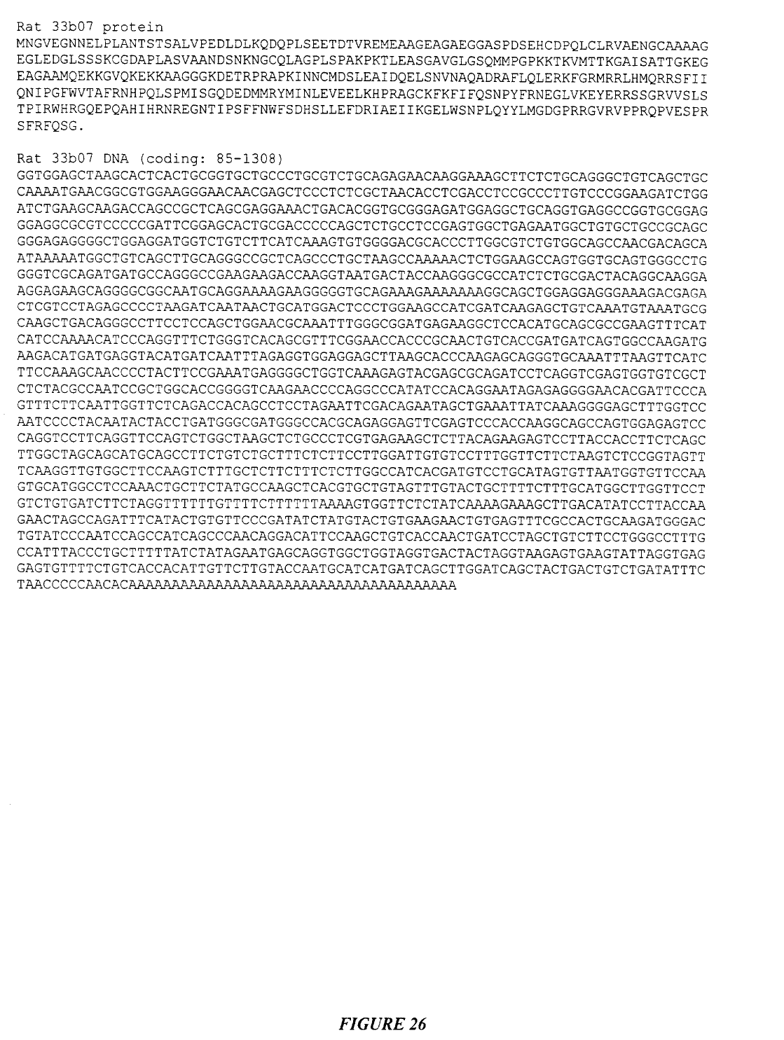

The nucleotide sequence of the full length rat 33b07 cDNA and the predicted amino acid sequence of the rat 33b07 polypeptide are shown in FIG. 26 and in SEQ ID NOs:52 and 53, respectively. The rat 33b07 cDNA encodes a protein having a molecular weight of approximately 44.7 kD and which is 407 amino acid residues in length. Rat 33b07 binds rKv4.3N and rKv4.2N with slight preference for rKv4.2N in yeast 2-hybrid assays.

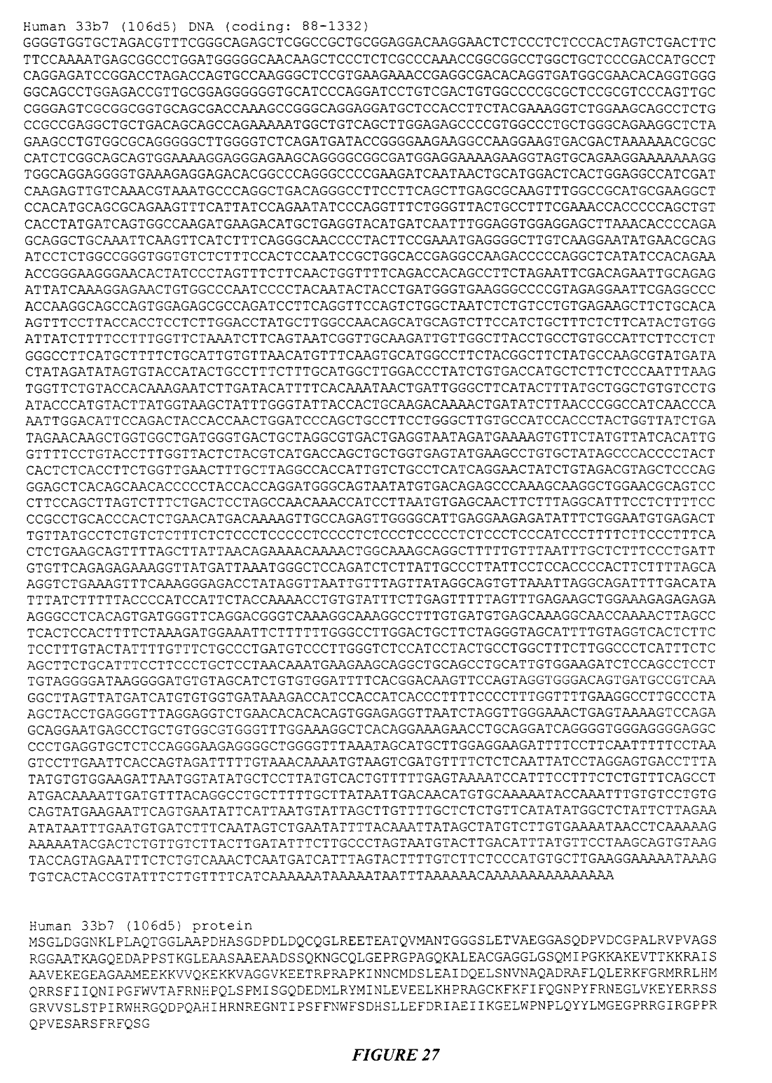

The nucleotide sequence of the full length human 33b07 cDNA and the predicted amino acid sequence of the human 33b07 polypeptide are shown in FIG. 27 and in SEQ ID NOs:54 and 55, respectively.

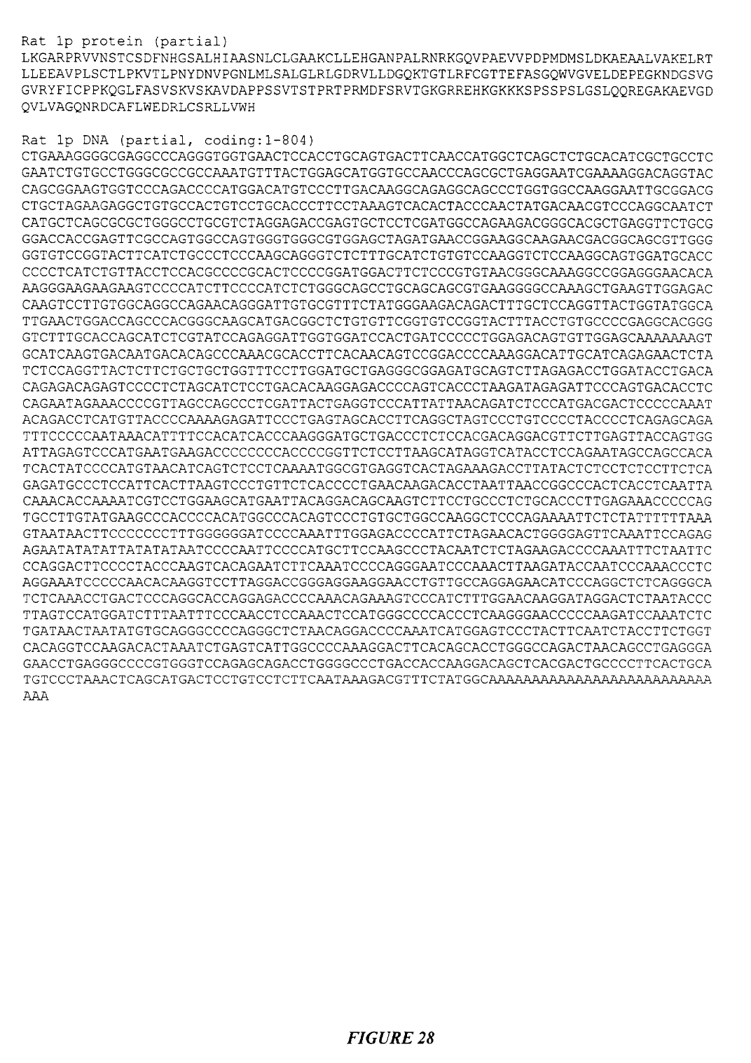

The nucleotide sequence of the partial length rat 1p cDNA and the predicted amino acid sequence of the rat 1p polypeptide are shown in FIG. 28 and in SEQ ID NOs:56 and 57, respectively. The rat 1p cDNA encodes a protein having a molecular weight of approximately 28.6 kD and which is 267 amino acid residues in length. Rat 1p binds rKv4.3N and rKv4.2N with slight preference for rKv4.3N in yeast two-hybrid assays.

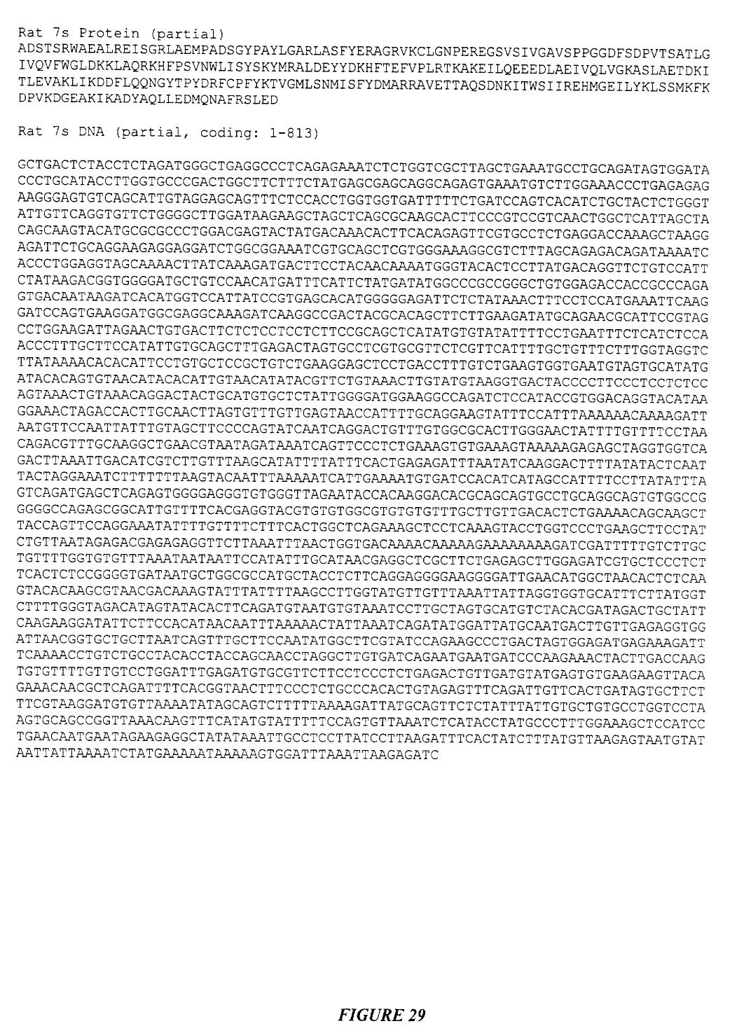

The nucleotide sequence of the partial length rat 7s cDNA and the predicted amino acid sequence of the rat 7s polypeptide are shown in FIG. 29 and in SEQ ID NOs:58 and 59, respectively. The rat 7s cDNA encodes a protein having a molecular weight of approximately 28.6 kD and which is 270 amino acid residues in length. Rat 7s binds rKv4.3N and rKv4.2N with preference for rKv4.3N in yeast two-hybrid assays.

The sequences of the PCIP molecules used in the methods of the present invention are summarized below, in Tables I and II.

TABLE I
PCIP Molecules Used in the Methods of the Present Invention

| | | | SEQ | SEQ | |
|---|---|---|---|---|---|
| | Nucleic Acid | | ID NO: | ID NO: | |
| PCIP | Molecule Form | Source | DNA | PROTEIN | ATCC |
| 1v | 1v | human | 1 | 2 | 98994 |
| | | (225-875)* | | | |
| | 1v | rat | 3 | 4 | 98946 |
| | | (210-860) | | | |
| | 1v | mouse | 5 | 6 | 98945 |
| | | (477-1127) | | | |
| | 1vl | rat | 7 | 8 | 98942 |
| | | (31-714) | | | |
| | 1vl | mouse | 9 | 10 | 98943 |
| | | (77-760) | | | |
| | 1vn | rat | 11 | 12 | 98944 |
| | (partial) | (345-955) | | | |
| 9q | Genomic DNA | human | 46 | | |
| | sequence | | | | |
| | (Exon 1 and | | | | |
| | flanking intron | | | | |
| | sequences) | | | | |
| | Genomic DNA | human | 47 | | |
| | sequence | | | | |
| | (Exons 2-11 | | | | |
| | and flanking | | | | |
| | intron | | | | |
| | sequences) | | | | |
| | 9ql | human | 13 | 14 | 98993 |
| | | (207-1019) | | | 98991 |
| | 9ql | rat (2-775) | 15 | 16 | 98948 |
| | (partial) | | | | |
| | 9ql | mouse | 17 | 18 | 98937 |
| | | (181-993) | | | |
| | 9qm | human | 19 | 20 | 98993 |
| | | (207-965) | | | 98991 |
| | 9qm | rat | 21 | 22 | 98941 |
| | | (214-972) | | | |
| | 9qs | human | 23 | 24 | 98951 |
| | | (207-869) | | | |
| | 9qs | monkey | 25 | 26 | 98950 |
| | | (133-795) | | | |
| | 9qc | rat | 27 | 28 | 98947 |
| | | (208-966) | | | |
| | 8t | rat | 29 | 30 | 98939 |
| | (partial) | (1-678) | | | |
| p19 | p19 | Human | 31 | 32 | |
| | | (1-771) | | | |
| | p19 | rat | 33 | 34 | 98936 |
| | (partial) | (1-330) | | | |
| | p19 | mouse | 35 | 36 | 98940 |
| | | (49-819) | | | |
| | p193 | Human | 39 | 40 | 98949 |
| | (partial) | (2-127) | | | |
| W28559 | W28559 | human | 37 | 38 | |
| | (partial) | (1-339) | | | |
| KChIP4 | KChIP4a | Monkey | 48 | 49 | |
| | | (265-966) | | | |
| | KChIP4b | Monkey | 50 | 51 | |
| | C-terminal | (265-966) | | | |
| | splice variant | | | | |
| | KChIP4c | Monkey | 69 | 70 | |
| | splice variant | (122-811) | | | |
| | KChIP4d | Monkey | 71 | 72 | |
| | splice variant | (64-816) | | | |

*The coordinates of the coding sequence are shown in parenthesis. The first column indicates the PCIPs which were identified and column 2 indicates the various nucleic acid forms identified for each PCIP.

TABLE II
PCIP Molecules Used in the Methods of the Present Invention

| | | | SEQ | SEQ | |
|---|---|---|---|---|---|
| | Nucleic Acid | | ID NO: | ID NO: | |
| PCIP | Molecule Form | Source | DNA | PROTEIN | ATCC |
| 33b07 | 33b07 | Human | 52 | 53 | |
| Novel | | (88-1332) | | | |
| | 33b07 | Rat | 54 | 55 | |
| | | (85-1308) | | | |
| 1p | 1p | Rat | 56 | 57 | |
| Novel | (partial) | (1-804) | | | |
| 7s | 7s | Rat | 58 | 59 | |
| Novel | (partial) | (1-813) | | | |
| 29x | 29x | Rat | 60 | 61 | |
| | | (433-1071) | | | |
| | 25r | Rat | 62 | | |
| | splice variant | (130-768) | | | |
| | of 29x | | | | |
| 5p | 5p | Rat | 63 | 64 | |
| | | (52-339) | | | |
| 7q | 7q | Rat | 65 | 66 | |
| | | (1-639) | | | |
| 19r | 19r | Rat | 67 | 68 | |
| | | (1-816) | | | |

*The coordinates of the coding sequence are shown in parenthesis. The first column indicates the four families of PCIPs which were identified and column 2 indicates the various nucleic acid forms identified for each family. Novel molecules are also indicated.

Plasmids containing the nucleotide sequences encoding human, rat and monkey PCIPs were deposited with American Type Culture Collection (ATCC), 10801 University Boulevard, Manassas, Va. 20110-2209, on Nov. 17, 1998, and assigned the Accession Numbers described above. These deposits will be maintained under the terms of the Budapest Treaty on the International Recognition of the Deposit of Microorganisms for the Purposes of Patent Procedure. These deposits were made merely as a convenience for those of skill in the art and are not an admission that a deposit is required under 35 U.S.C. §112.

Clones containing cDNA molecules encoding human p19 (clone EphP19) and human 33b07 (clone Eph33b07) were deposited with American Type Culture Collection (Manassas, Va.) on Jul. 8, 1998 as Accession Number ______, as part of a composite deposit representing a mixture of two strains, each carrying one recombinant plasmid harboring a particular cDNA clone. (The ATCC strain designation for the mixture of hP19 and h33b07 is EphP19h33b07mix).

To distinguish the strains and isolate a strain harboring a particular cDNA clone, an aliquot of the mixture can be streaked out to single colonies on LB plates supplemented with 100 ug/ml ampicillin, single colonies grown, and then plasmid DNA extracted using a standard minipreparation procedure. Next, a sample of the DNA minipreparation can be digested with NotI and the resultant products resolved on a 0.8% agarose gel using standard DNA electrophoresis conditions. The digest gives the following band patterns: EphP19: 7 kb 9 (single band), Eph33b07: 5.8 kb (single band).

Various aspects of the invention are described in further detail in the following subsections:

I. Screening Assays:

The invention provides a method (also referred to herein as a “screening assay”) for identifying modulators, i.e., candidate or test compounds or agents (e.g., peptides, peptidomimetics, small molecules or other drugs) which bind to PCIP proteins, have a stimulatory or inhibitory effect on, for example, PCIP expression or PCIP activity, or have a stimulatory or inhibitory effect on, for example, the expression or activity of a PCIP substrate.

In one embodiment, the invention provides assays for screening candidate or test compounds which are substrates of a PCIP protein or polypeptide or biologically active portion thereof. In another embodiment, the invention provides assays for screening candidate or test compounds which bind to or modulate the activity of a PCIP protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner USP '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

In one embodiment, an assay is a cell-based assay in which a cell which expresses a PCIP protein or biologically active portion thereof is contacted with a test compound and the ability of the test compound to modulate PCIP activity, e.g., binding to a potassium channel comprising a Kv4.2 or Kv4.2 subunit, or a portion thereof, is determined. Determining the ability of the test compound to modulate PCIP activity can be accomplished by monitoring, for example, the Iₜₒ current or the release of a neurotransmitter from a cell which expresses PCIP such as a cardiac cell. Currents in cells, e.g., the Iₜₒ current, can be measured using the patch-clamp technique as described in the Examples section using the techniques described in, for example, Hamill et al. 1981. Pfluegers Arch. 391:85-100). The cell, for example, can be of mammalian origin. Determining the ability of the test compound to modulate the ability of PCIP to bind to a substrate can be accomplished, for example, by coupling the PCIP substrate with a radioisotope or enzymatic label such that binding of the PCIP substrate to PCIP can be determined by detecting the labeled PCIP substrate in a complex. For example, compounds (e.g., PCIP substrates) can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound (e.g., PCIP substrate) to interact with PCIP without the labeling of any of the interactants. For example, a microphysiometer can be used to detect the interaction of a compound with PCIP without the labeling of either the compound or the PCIP. McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and PCIP.

In another embodiment, an assay is a cell-based assay comprising contacting a cell expressing a PCIP target molecule (e.g., a potassium channel comprising a Kv4.2 or Kv4.2 subunit, or a portion thereof, is determined. Determining the ability of the test compound to modulate, or a fragment thereof) with a test compound and determining the ability of the test compound to modulate (e.g., stimulate or inhibit) the activity of the PCIP target molecule. Determining the ability of the test compound to modulate the activity of a PCIP target molecule can be accomplished, for example, by determining the ability of the PCIP protein to bind to or interact with the PCIP target molecule, e.g., a potassium channel or a fragment thereof.

Determining the ability of the PCIP protein or a biologically active fragment thereof, to bind to or interact with a PCIP target molecule can be accomplished by one of the methods described above for determining direct binding. In a preferred embodiment, determining the ability of the PCIP protein to bind to or interact with a PCIP target molecule can be accomplished by determining the activity of the target molecule. For example, the activity of the target molecule can be determined by detecting induction of a cellular second messenger of the target (i.e., intracellular Ca²⁺, diacylglycerol, IP₃, and the like), detecting catalytic/enzymatic activity of the target an appropriate substrate, detecting the induction of a reporter gene (comprising a target-responsive regulatory element operatively linked to a nucleic acid encoding a detectable marker, e.g., luciferase), or detecting a target-regulated cellular response such as the release of a neurotransmitter.

In yet another embodiment, an assay of the present invention is a cell-free assay in which a PCIP protein or biologically active portion thereof is contacted with a test compound and the ability of the test compound to bind to the PCIP protein or biologically active portion thereof is determined. Preferred biologically active portions of the PCIP proteins to be used in assays of the present invention include fragments which participate in interactions with non-PCIP molecules, e.g., potassium channels comprising a Kv4.2 or Kv4.2 subunit, or a portion thereof, is determined. Determining the ability of the test compound to modulate, or fragments thereof, or fragments with high surface probability scores. Binding of the test compound to the PCIP protein can be determined either directly or indirectly as described above. In a preferred embodiment, the assay includes contacting the PCIP protein or biologically active portion thereof with a known compound which binds PCIP to form an assay mixture, contacting the assay mixture with a test compound, and determining the ability of the test compound to interact with a PCIP protein, wherein determining the ability of the test compound to interact with a PCIP protein comprises determining the ability of the test compound to preferentially bind to PCIP or biologically active portion thereof as compared to the known compound.

In another embodiment, the assay is a cell-free assay in which a PCIP protein or biologically active portion thereof is contacted with a test compound and the ability of the test compound to modulate (e.g., stimulate or inhibit) the activity of the PCIP protein or biologically active portion thereof is determined. Determining the ability of the test compound to modulate the activity of a PCIP protein can be accomplished, for example, by determining the ability of the PCIP protein to bind to a PCIP target molecule by one of the methods described above for determining direct binding. Determining the ability of the PCIP protein to bind to a PCIP target molecule can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In an alternative embodiment, determining the ability of the test compound to modulate the activity of a PCIP protein can be accomplished by determining the ability of the PCIP protein to further modulate the activity of a downstream effector of a PCIP target molecule. For example, the activity of the effector molecule on an appropriate target can be determined or the binding of the effector to an appropriate target can be determined as previously described.

In yet another embodiment, the cell-free assay involves contacting a PCIP protein or biologically active portion thereof with a known compound which binds the PCIP protein to form an assay mixture, contacting the assay mixture with a test compound, and determining the ability of the test compound to interact with the PCIP protein, wherein determining the ability of the test compound to interact with the PCIP protein comprises determining the ability of the PCIP protein to preferentially bind to or modulate the activity of a PCIP target molecule.

The cell-free assays of the present invention are amenable to use of both soluble and/or membrane-bound forms of isolated proteins. In the case of cell-free assays in which a membrane-bound form of an isolated protein is used (e.g., a potassium channel) it may be desirable to utilize a solubilizing agent such that the membrane-bound form of the isolated protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-1214, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

In more than one embodiment of the above assay methods of the present invention, it may be desirable to immobilize either PCIP or its target molecule to facilitate separation of complexed from uncomplexed forms of one or both of the proteins, as well as to accommodate automation of the assay. Binding of a test compound to a PCIP protein, or interaction of a PCIP protein with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/PCIP fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or PCIP protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of PCIP binding or activity determined using standard techniques.

Other techniques for immobilizing proteins on matrices can also be used in the screening assays of the invention. For example, either a PCIP protein or a PCIP target molecule can be immobilized utilizing conjugation of biotin and streptavidin. Biotinylated PCIP protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies reactive with PCIP protein or target molecules but which do not interfere with binding of the PCIP protein to its target molecule can be derivatized to the wells of the plate, and unbound target or PCIP protein trapped in the wells by antibody conjugation. Methods for detecting such complexes, in addition to those described above for the GST-immobilized complexes, include immunodetection of complexes using antibodies reactive with the PCIP protein or target molecule, as well as enzyme-linked assays which rely on detecting an enzymatic activity associated with the PCIP protein or target molecule.

In a preferred embodiment, candidate or test compounds or agents are tested for their ability to inhibit or stimulate a PCIP molecule's ability to modulate vesicular traffic and protein transport in a cell, e.g., a cardiac cell, using the assays described in, for example, Komada M. et al. (1999) Genes Dev.13(11):1475-85, and Roth M. G. et al. (1999) Chem. Phys. Lipids. 98(1-2):141-52, the contents of which are incorporated herein by reference.

In another preferred embodiment, candidate or test compounds or agents are tested for their ability to inhibit or stimulate a PCIP molecule's ability to regulate the phosphorylation state of a potassium channel protein or portion thereof, using for example, an in vitro kinase assay. Briefly, a PCIP target molecule, e.g., an immunoprecipitated potassium channel from a cell line expressing such a molecule, can be incubated with the PCIP protein and radioactive ATP, e.g., [γ-³²P] ATP, in a buffer containing MgCl₂ and MnCl₂, e.g., 10 mM MgCl₂ and 5 mM MnCl₂. Following the incubation, the immunoprecipitated PCIP target molecule, e.g., the potassium channel, can be separated by SDS-polyacrylamide gel electrophoresis under reducing conditions, transferred to a membrane, e.g., a PVDF membrane, and autoradiographed. The appearance of detectable bands on the autoradiograph indicates that the PCIP substrate, e.g., the potassium channel, has been phosphorylated. Phosphoaminoacid analysis of the phosphorylated substrate can also be performed in order to determine which residues on the PCIP substrate are phosphorylated. Briefly, the radiophosphorylated protein band can be excised from the SDS gel and subjected to partial acid hydrolysis. The products can then be separated by one-dimensional electrophoresis and analyzed on, for example, a phosphoimager and compared to ninhydrin-stained phosphoaminoacid standards. Assays such as those described in, for example, Tamaskovic R. et al. (1999) Biol. Chem. 380(5):569-78, the contents of which are incorporated herein by reference, can also be used.

In another preferred embodiment, candidate or test compounds or agents are tested for their ability to inhibit or stimulate a PCIP molecule's ability to associate with (e.g., bind) calcium, using for example, the assays described in Liu L. (1999) Cell Signal. 11 (5):3 17-24 and Kawai T. et al. (1999) Oncogene 18(23):3471-80, the contents of which are incorporated herein by reference.

In another preferred embodiment, candidate or test compounds or agents are tested for their ability to inhibit or stimulate a PCIP molecule's ability to modulate chromatin formation in a cell, using for example, the assays described in Okuwaki M. et al. (1998) J. Biol. Chem. 273(51):34511-8 and Miyaji-Yamaguchi M. (1999) J. Mol. Biol. 290(2):547-557, the contents of which are incorporated herein by reference.

In yet another preferred embodiment, candidate or test compounds or agents are tested for their ability to inhibit or stimulate a PCIP molecule's ability to modulate cellular proliferation, using for example, the assays described in Baker F. L. et al. (1995) Cell Prolif. 28(1):1-15, Cheviron N. et al. (1996) Cell Prolif. 29(8):437-46, Hu Z. W. et al. (1999) J. Pharmacol. Exp. Ther. 290(1):28-37 and Elliott K. et al. (1999) Oncogene 18(24):3564-73, the contents of which are incorporated herein by reference.

In a preferred embodiment, candidate or test compounds or agents are tested for their ability to inhibit or stimulate a PCIP molecule's ability to regulate the association of a potassium channel protein or portion thereof with the cellular cytoskeleton, using for example, the assays described in Gonzalez C. et al. (1998) Cell Mol. Biol. 44(7):1117-27and Chia C. P. et al. (1998) Exp. Cell Res.244(1):340-8, the contents of which are incorporated herein by reference.

In another preferred embodiment, candidate or test compounds or agents are tested for their ability to inhibit or stimulate a PCIP molecule's ability to modulate membrane excitability, using for example, the assays described in Bar-Sagi D. et al. (1985) J. Biol. Chem. 260(8):4740-4 and Barker J. L. et al. (1984) Neurosci. Lett. 47(3):313-8, the contents of which are incorporated herein by reference.

In another preferred embodiment, candidate or test compounds or agents are tested for their ability to inhibit or stimulate a PCIP molecule's ability to modulate cytokine signaling in a cell, e.g., a cardiac cell, the assays described in Nakashima Y. et al. (1999) J. Bone Joint Surg. Am. 81(5):603-15, the contents of which are incorporated herein by reference.

In another embodiment, modulators of PCIP expression are identified in a method wherein a cell is contacted with a candidate compound and the expression of PCIP mRNA or protein in the cell is determined. The level of expression of PCIP mRNA or protein in the presence of the candidate compound is compared to the level of expression of PCIP mRNA or protein in the absence of the candidate compound. The candidate compound can then be identified as a modulator of PCIP expression based on this comparison. For example, when expression of PCIP mRNA or protein is greater (statistically significantly greater) in the presence of the candidate compound than in its absence, the candidate compound is identified as a stimulator of PCIP mRNA or protein expression. Alternatively, when expression of PCIP mRNA or protein is less (statistically significantly less) in the presence of the candidate compound than in its absence, the candidate compound is identified as an inhibitor of PCIP mRNA or protein expression. The level of PCIP mRNA or protein expression in the cells can be determined by methods described herein for detecting PCIP mRNA or protein.

In yet another aspect of the invention, the PCIP proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with PCIP (“PCIP-binding proteins” or “PCIP-bp”) and are involved in PCIP activity (described in more detail in the Examples section below). Such PCIP-binding proteins are also likely to be involved in the propagation of signals by the PCIP proteins or PCIP targets as, for example, downstream elements of a PCIP-mediated signaling pathway. Alternatively, such PCIP-binding proteins are likely to be PCIP inhibitors.

The two-hybrid system is based on the modular nature of most transcription factors, which consist of separable DNA-binding and activation domains. Briefly, the assay utilizes two different DNA constructs. In one construct, the gene that codes for a PCIP protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a PCIP-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the PCIP protein.

This invention further pertains to novel agents identified by the above-described screening assays. Accordingly, it is within the scope of this invention to further use an agent identified as described herein in an appropriate animal model. For example, an agent identified as described herein (e.g., a PCIP modulating agent, an antisense PCIP nucleic acid molecule, a PCIP-specific antibody, or a PCIP-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

II. Predictive Medicine:

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining PCIP protein and/or nucleic acid expression as well as PCIP activity, in the context of a biological sample (e.g., blood, serum, cells, tissue) to thereby determine whether an individual is afflicted with a disease or disorder, or is at risk of developing a disorder, associated with aberrant PCIP expression or activity. The invention also provides for prognostic (or predictive) assays for determining whether an individual is at risk of developing a disorder associated with PCIP protein, nucleic acid expression or activity. For example, mutations in a PCIP gene can be assayed in a biological sample. Such assays can be used for prognostic or predictive purpose to thereby phophylactically treat an individual prior to the onset of a disorder characterized by or associated with PCIP protein, nucleic acid expression or activity.

Another aspect of the invention pertains to monitoring the influence of agents (e.g., drugs, compounds) on the expression or activity of PCIP in clinical trials.

These and other agents are described in further detail in the following sections.

1. Diagnostic Assays

An exemplary method for detecting the presence or absence of PCIP protein or nucleic acid in a biological sample involves obtaining a biological sample from a test subject and contacting the biological sample with a compound or an agent capable of detecting PCIP protein or nucleic acid (e.g., mRNA, genomic DNA) that encodes PCIP protein such that the presence of PCIP protein or nucleic acid is detected in the biological sample. A preferred agent for detecting PCIP mRNA or genomic DNA is a labeled nucleic acid probe capable of hybridizing to PCIP mRNA or genomic DNA. The nucleic acid probe can be, for example, a full-length PCIP nucleic acid, such as the nucleic acid of SEQ ID NO:1, SEQ ID NO:3 SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:46, SEQ ID NO:47, SEQ ID NO:48, SEQ ID NO:50, SEQ ID NO:52, SEQ ID NO:54, SEQ ID NO:56, SEQ ID NO:58, SEQ ID NO:69, or SEQ ID NO:71, or the DNA insert of the plasmid deposited with ATCC as Accession Number 98936, 98937, 98938, 98939, 98940, 98941, 98942, 98943, 98944, 98945, 98946, 98947, 98948, 98949, 98950, 98951, 98991, 98993, or 98994, or a portion thereof, such as an oligonucleotide of at least 15, 30, 50, 100, 250 or 500 nucleotides in length and sufficient to specifically hybridize under stringent conditions to PCIP mRNA or genomic DNA. Other suitable probes for use in the diagnostic assays of the invention are described herein.

A preferred agent for detecting PCIP protein is an antibody capable of binding to PCIP protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect PCIP mRNA, protein, or genomic DNA in a biological sample in vitro as well as in vivo. For example, in vitro techniques for detection of PCIP mRNA include Northern hybridizations and in situ hybridizations. In vitro techniques for detection of PCIP protein include enzyme linked immunosorbent assays (ELISAs), Western blots, immunoprecipitations and immunofluorescence. In vitro techniques for detection of PCIP genomic DNA include Southern hybridizations. Furthermore, in vivo techniques for detection of PCIP protein include introducing into a subject a labeled anti-PCIP antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a serum sample isolated by conventional means from a subject.

In another embodiment, the methods further involve obtaining a control biological sample from a control subject, contacting the control sample with a compound or agent capable of detecting PCIP protein, mRNA, or genomic DNA, such that the presence of PCIP protein, mRNA or genomic DNA is detected in the biological sample, and comparing the presence of PCIP protein, mRNA or genomic DNA in the control sample with the presence of PCIP protein, mRNA or genomic DNA in the test sample.

The invention also encompasses kits for detecting the presence of PCIP in a biological sample. For example, the kit can comprise a labeled compound or agent capable of detecting PCIP protein or mRNA in a biological sample; means for determining the amount of PCIP in the sample; and means for comparing the amount of PCIP in the sample with a standard. The compound or agent can be packaged in a suitable container. The kit can further comprise instructions for using the kit to detect PCIP protein or nucleic acid.

2. Prognostic Assays

The diagnostic methods described herein can furthermore be utilized to identify subjects having or at risk of developing a disease or disorder associated with aberrant PCIP expression or activity. For example, the assays described herein, such as the preceding diagnostic assays or the following assays, can be utilized to identify a subject having or at risk of developing a disorder associated with a misregulation in PCIP protein activity or nucleic acid expression, such as a cardiovascular disorders such as sinus node disfunction, angina, heart failure, hypertension, atrial fibrillation, atrial flutter, dilated cardiomyopathy, idiopathic cardiomyopathy, myocardial infarction, coronary artery disease, coronary artery spasm, or arrythmia.

Alternatively, the prognostic assays can be utilized to identify a subject having or at risk for developing a disorder associated with a misregulation in PCIP protein activity or nucleic acid expression, such as a potassium channel associated disorder. Thus, the present invention provides a method for identifying a disease or disorder associated with aberrant PCIP expression or activity in which a test sample is obtained from a subject and PCIP protein or nucleic acid (e.g., mRNA or genomic DNA) is detected, wherein the presence of PCIP protein or nucleic acid is diagnostic for a subject having or at risk of developing a disease or disorder associated with aberrant PCIP expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant PCIP expression or activity. For example, such methods can be used to determine whether a subject can be effectively treated with an agent for a cardiovascular disorder. Thus, the present invention provides methods for determining whether a subject can be effectively treated with an agent for a disorder associated with aberrant PCIP expression or activity in which a test sample is obtained and PCIP protein or nucleic acid expression or activity is detected (e.g., wherein the abundance of PCIP protein or nucleic acid expression or activity is diagnostic for a subject that can be administered the agent to treat a disorder associated with aberrant PCIP expression or activity).

The methods of the invention can also be used to detect genetic alterations in a PCIP gene, thereby determining if a subject with the altered gene is at risk for a disorder characterized by misregulation in PCIP protein activity or nucleic acid expression, such as a cardiovascular disorder. In preferred embodiments, the methods include detecting, in a sample of cells from the subject, the presence or absence of a genetic alteration characterized by at least one of an alteration affecting the integrity of a gene encoding a PCIP-protein, or the mis-expression of the PCIP gene. For example, such genetic alterations can be detected by ascertaining the existence of at least one of 1) a deletion of one or more nucleotides from a PCIP gene; 2) an addition of one or more nucleotides to a PCIP gene; 3) a substitution of one or more nucleotides of a PCIP gene, 4) a chromosomal rearrangement of a PCIP gene; 5) an alteration in the level of a messenger RNA transcript of a PCIP gene, 6) aberrant modification of a PCIP gene, such as of the methylation pattern of the genomic DNA, 7) the presence of a non-wild type splicing pattern of a messenger RNA transcript of a PCIP gene, 8) a non-wild type level of a PCIP-protein, 9) allelic loss of a PCIP gene, and 10) inappropriate post-translational modification of a PCIP-protein. As described herein, there are a large number of assays known in the art which can be used for detecting alterations in a PCIP gene. A preferred biological sample is a tissue or serum sample isolated by conventional means from a subject.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in the PCIP-gene (see Abravaya et al. (1995) Nucleic Acids Res .23:675-682). This method can include the steps of collecting a sample of cells from a subject, isolating nucleic acid (e.g., genomic, mRNA or both) from the cells of the sample, contacting the nucleic acid sample with one or more primers which specifically hybridize to a PCIP gene under conditions such that hybridization and amplification of the PCIP-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al., (1990) Proc. Natl. Acad. Sci. USA 87:1874-1878), transcriptional amplification system (Kwoh, D. Y. et al., (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a PCIP gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in PCIP can be identified by hybridizing a sample and control nucleic acids, e.g., DNA or RNA, to high density arrays containing hundreds or thousands of oligonucleotides probes (Cronin, M. T. et al. (1996) Human Mutation 7: 244-255; Kozal, M. J. et al. (1996) Nature Medicine 2: 753-759). For example, genetic mutations in PCIP can be identified in two dimensional arrays containing light-generated DNA probes as described in Cronin, M. T. et al. supra Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the PCIP gene and detect mutations by comparing the sequence of the sample PCIP with the corresponding wild-type (control) sequence. Examples of sequencing reactions include those based on techniques developed by Maxam and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the PCIP gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the wild-type PCIP sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl Acad Sci USA 85:4397; Saleebaet al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in PCIP cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a PCIP sequence, e.g., a wild-type PCIP sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in PCIP genes. For example, single strand conformation polymorphism (SSCP) may be used to detect differences in electrophoretic mobility between mutant and wild type nucleic acids (Orita et al. (1989) Proc Natl. Acad. Sci USA: 86:2766, see also Cotton (1993) Mutat. Res. 285:125-144; and Hayashi (1992) Genet. Anal. Tech. Appl. 9:73-79). Single-stranded DNA fragments of sample and control PCIP nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a PCIP gene.

Furthermore, any cell type or tissue in which PCIP is expressed may be utilized in the prognostic assays described herein.

3. Monitoring of Effects during Clinical Trials

Monitoring the influence of agents (e.g., drugs) on the expression or activity of a PCIP protein (e.g., the modulation of membrane excitability or resting potential) can be applied not only in basic drug screening, but also in clinical trials. For example, the effectiveness of an agent determined by a screening assay as described herein to increase PCIP gene expression, protein levels, or upregulate PCIP activity, can be monitored in clinical trials of subjects exhibiting decreased PCIP gene expression, protein levels, or downregulated PCIP activity. Alternatively, the effectiveness of an agent determined by a screening assay to decrease PCIP gene expression, protein levels, or downregulate PCIP activity, can be monitored in clinical trials of subjects exhibiting increased PCIP gene expression, protein levels, or upregulated PCIP activity. In such clinical trials, the expression or activity of a PCIP gene, and preferably, other genes that have been implicated in, for example, a potassium channel associated disorder can be used as a “read out” or markers of the phenotype of a particular cell.

For example, and not by way of limitation, genes, including PCIP, that are modulated in cells by treatment with an agent (e.g., compound, drug or small molecule) which modulates PCIP activity (e.g., identified in a screening assay as described herein) can be identified. Thus, to study the effect of agents on potassium channel associated disorders, for example, in a clinical trial, cells can be isolated and RNA prepared and analyzed for the levels of expression of PCIP and other genes implicated in the potassium channel associated disorder, respectively. The levels of gene expression (e.g., a gene expression pattern) can be quantified by northern blot analysis or RT-PCR, as described herein, or alternatively by measuring the amount of protein produced, by one of the methods as described herein, or by measuring the levels of activity of PCIP or other genes. In this way, the gene expression pattern can serve as a marker, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) including the steps of (i) obtaining a pre-administration sample from a subject prior to administration of the agent; (ii) detecting the level of expression of a PCIP protein, mRNA, or genomic DNA in the preadministration sample; (iii) obtaining one or more post-administration samples from the subject; (iv) detecting the level of expression or activity of the PCIP protein, mRNA, or genomic DNA in the post-administration samples; (v) comparing the level of expression or activity of the PCIP protein, mRNA, or genomic DNA in the pre-administration sample with the PCIP protein, mRNA, or genomic DNA in the post administration sample or samples; and (vi) altering the administration of the agent to the subject accordingly. For example, increased administration of the agent may be desirable to increase the expression or activity of PCIP to higher levels than detected, i.e., to increase the effectiveness of the agent. Alternatively, decreased administration of the agent may be desirable to decrease expression or activity of PCIP to lower levels than detected, i.e. to decrease the effectiveness of the agent. According to such an embodiment, PCIP expression or activity may be used as an indicator of the effectiveness of an agent, even in the absence of an observable phenotypic response.

III. Methods of Treatment:

The present invention provides for both prophylactic and therapeutic methods of treating a subject at risk of (or susceptible to) a disorder or having a disorder associated with aberrant PCIP expression or activity such as a cardiovascular disorder. With regard to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”.) Thus, another aspect of the invention provides methods for tailoring an individual's prophylactic or therapeutic treatment with either the PCIP molecules of the present invention or PCIP modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

1. Prophylactic Methods

In one aspect, the invention provides a method for preventing in a subject, a disease or condition associated with an aberrant PCIP expression or activity such as a cardiovascular disorder, by administering to the subject a PCIP or an agent which modulates PCIP expression or at least one PCIP activity. Subjects at risk for a disease which is caused or contributed to by aberrant PCIP expression or activity can be identified by, for example, any or a combination of diagnostic or prognostic assays as described herein. Administration of a prophylactic agent can occur prior to the manifestation of symptoms characteristic of the PCIP aberrancy, such that a disease or disorder is prevented or, alternatively, delayed in its progression. Depending on the type of PCIP aberrancy, for example, a PCIP, PCIP agonist or PCIP antagonist agent can be used for treating the subject. The appropriate agent can be determined based on screening assays described herein.

2. Therapeutic Methods

Another aspect of the invention pertains to methods of modulating PCIP expression or activity for therapeutic purposes. Accordingly, in an exemplary embodiment, the modulatory method of the invention involves contacting a cell with a PCIP or agent that modulates one or more of the activities of PCIP protein activity associated with the cell. An agent that modulates PCIP protein activity can be an agent as described herein, such as a nucleic acid or a protein, a naturally-occurring target molecule of a PCIP protein (e.g., a PCIP substrate), a PCIP antibody, a PCIP agonist or antagonist, a peptidomimetic of a PCIP agonist or antagonist, or other small molecule. In one embodiment, the agent stimulates one or more PCIP activities. Examples of such stimulatory agents include active PCIP protein and a nucleic acid molecule encoding PCIP that has been introduced into the cell. In another embodiment, the agent inhibits one or more PCIP activities. Examples of such inhibitory agents include antisense PCIP nucleic acid molecules, anti-PCIP antibodies, and PCIP inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant expression or activity of a PCIP protein or nucleic acid molecule. Examples of such disorders include cardiovascular disorders such as long-QT syndrome, sinus node disfunction, angina, heart failure, hypertension, atrial fibrillation, atrial flutter, dilated cardiomyopathy, idiopathic cardiomyopathy, myocardial infarction, coronary artery disease, coronary artery spasm, or arrythmia. In one embodiment, the method involves administering an agent (e.g., an agent identified by a screening assay described herein), or combination of agents that modulates (e.g., upregulates or downregulates) PCIP expression or activity. In another embodiment, the method involves administering a PCIP protein or nucleic acid molecule as therapy to compensate for reduced or aberrant PCIP expression or activity.

Stimulation of PCIP activity is desirable in situations in which PCIP is abnormally downregulated and/or in which increased PCIP activity is likely to have a beneficial effect. For example, stimulation of PCIP activity is desirable in situations in which a PCIP is downregulated and/or in which increased PCIP activity is likely to have a beneficial effect. Likewise, inhibition of PCIP activity is desirable in situations in which PCIP is abnormally upregulated and/or in which decreased PCIP activity is likely to have a beneficial effect.

A PCIP molecule or an agent that modulates one or more of the activities of PCIP protein activity associated with the cell can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition used in the methods of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a fragment of a PCIP protein or an anti-PCIP antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical compositions used in the methods of the invention can also be prepared in the form of suppositories (e.g., with conventional suppository bases such as cocoa butter and other glycerides) or retention enemas for rectal delivery.

In one embodiment, pharmaceutical compositions used in the methods of the invention are prepared with carriers that will protect the active compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The methods of the present invention encompasses the use of agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha.-interferon, .beta.-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to Form an antibody heteroconjugate as described by Segal in U.S. Pat. No. 4,676,980.

The nucleic acid molecules used in the methods of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

3. Pharmacogenomics

The PCIP molecules of the present invention, as well as agents, or modulators which have a stimulatory or inhibitory effect on PCIP activity (e.g., PCIP gene expression) as identified by a screening assay described herein can be administered to individuals to treat (prophylactically or therapeutically) potassium channel associated disorders associated with aberrant PCIP activity (e.g, cardiovascular disorders such as long-QT syndrome, sinus node disfunction, angina, heart failure, hypertension, atrial fibrillation, atrial flutter, dilated cardiomyopathy, idiopathic cardiomyopathy, myocardial infarction, coronary artery disease, coronary artery spasm, or arrythmia). In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a PCIP molecule or PCIP modulator as well as tailoring the dosage and/or therapeutic regimen of treatment with a PCIP molecule or PCIP modulator.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11):983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drugs target is known (e.g., a PCIP protein of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a drug (e.g., a PCIP molecule or PCIP modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with a PCIP molecule or PCIP modulator, such as a modulator identified by one of the exemplary screening assays described herein.

This invention is further illustrated by the following examples which should not be construed as limiting. The contents of all references, patents and published patent applications cited throughout this application, as well as the Figures and the Sequence Listing are incorporated herein by reference.

### EXAMPLES

The following materials and methods were used in the Examples.

Strains, Plasmids, Bait cDNAs, and General Microbiological Techniques

Basic yeast strains (HF7c, Y187,) bait (pGBT9) and fish (pACT2) plasmids used in this work were purchased from Clontech (Palo Alto, Calif.). cDNAs encoding rat Kv4.3, Kv4.2, and Kv1.1, were provided by Wyeth-Ayerst Research (865 Ridge Rd., Monmouth Junction, N.J. 08852) Standard yeast media including synthetic complete medium lacking L-leucine, L-tryptophan, and L-histidine were prepared and yeast genetic manipulations were performed as described (Sherman (1991) Meth. Enzymol. 194:3-21). Yeast transformations were performed using standard protocols (Gietz et al. (1992) Nucleic Acids Res. 20:1425; Ito et al (1983) J. Bacteriol. 153:163-168). Plasmid DNAs were isolated from yeast strains by a standard method (Hoffinan and Winston (1987) Gene 57:267-272).

Bait and Yeast Strain Construction

The first 180 amino acids of rKv4.3 (described in Serdio P. et al. (1996) J. Neurophys 75:2174-2179) were amplified by PCR and cloned in frame into pGBT9 resulting in plasmid pFWA2, (hereinafter “bait”). This bait was transformed into the two-hybrid screening strain HF7c and tested for expression and self-activation. The bait was validated for expression by Western blotting. The rKv4.3 bait did not self-activate in the presence of 10 mM 3-amino-1,2,3-Triazole (3-AT).

Library Construction

Rat mid brain tissue was provided by Wyeth-Ayerst Research (Monmouth Junction, N.J.). Total cellular RNA was extracted from the tissues using standard techniques (Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., (1989)). mRNA was prepared using a Poly-A Spin mRNA Isolation Kit from New England Biolabs (Beverly, Mass.). cDNA from the mRNA sample was synthesized using a cDNA Synthesis Kit from Stratagene (La Jolla, Calif.) and ligated into pACT2's EcoRi and XhoI sites, giving rise to a two-hybrid library.

Two-hybrid Screening

Two-hybrid screens were carried out essentially as described in Bartel, P. et al. (1993) “Using the Two-Hybrid System to Detect Polypeptide-Polypeptide Interactions” in Cellular Interactions in Development: A Practical Approach, Hartley, D. A. ed. Oxford University Press, Oxford, pp. 153-179, with a bait-library pair of rkv4.3 bait-rat mid brain library. A filter disk beta-galactosidase (beta-gal) assay was performed essentially as previously described (Brill et al. (1994) Mol. Biol. Cell. 5:297-312). Clones that were positive for both reporter gene activity (His and beta-galactosidase) were scored and fish, plasmids were isolated from yeast, transformed into E. coli strain KC8, DNA plasmids were purified and the resulting plasmids were sequenced by conventional methods (Sanger F. et al. (1977) PNAS, 74:5463-67).

Specificity Test

Positive interactor clones were subjected to a binding specificity test where they were exposed to a panel of related and unrelated baits by a mating scheme previously described (Finley R. L. Jr. et al. (1994) PNAS, 91(26):12980-12984). Briefly, positive fish plasmids were transformed into Y187 and the panel of baits were transformed into HF7c. Transformed fish and bait cells were streaked out as stripes on selective medium plates, mated on YPAD plates, and tested for reporter gene activity.

Analysis

PCIP nuleotides were analyzed for nucleic acid hits by the BLASTN 1.4.8MP program (Altschul et al. (1990) Basic Local Alignment Search Tool. J. Mol. Biol. 215:403-410). PCIP proteins were analyzed for polypeptide hits by the BLASTP 1.4.9MP program.

### Example 1

### Identification of Rat PCIP cDNAs

The Kv4.3 gene coding sequence (coding for the first 180 amino acids) was amplified by PCR and cloned into pGBT9 creating a GAL4 DNA-binding domain-Kv4.3(1-180) gene fusion (plasmid pFWA2). HF7c was transformed with this construct. The resulting strain grew on synthetic complete medium lacking L-tryptophan but not on synthetic complete medium lacking L-tryptophan and L-histidine in the presence of 10 mM 3-AT demonstrating that the {GAL4 DNA-binding domain}-{vKv4.3(1-180)} gene fusion does not have intrinsic transcriptional activation activity higher than the threshhold allowed by 10 mM 3-AT.

In this example, a yeast two-hybrid assay was performed in which a plasmid containing a {GAL4 DNA-binding domain}-{rKv4.3(1-180)} gene fusion was introduced into the yeast two-hybrid screening strain HF7c described above. HF7c was then transformed with the rat mid brain two-hybrid library. Approximately six million transformants were obtained and plated in selection medium. Colonies that grew in the selection medium and expressed the beta-galactosidase reporter gene were further characterized and subjected to retransformation and specificity assays. The retransformation and specificity tests yielded three PCIP clones (rat 1v, 8t, and 9qm) that were able to bind to the Kv4.3 polypeptide.

The full length sequences for the rat 1v gene, and partial sequences for 8t and 9q genes were derived as follows. The partial rat PCIP sequences were used to prepare probes, which were then used to screen, for example, rat mid brain cDNA libraries. Positive clones were identified, amplified and sequenced using standard techniques, to obtain the full length sequence. Additionally, a rapid amplification of the existing rat PCIP cDNA ends (using for example, 5′ RACE, by Gibco, BRL) was used to complete the 5′ end of the transcript.

### Example 2

### Identification of Human 1v cDNA

To obtain the human 1v nucleic acid molecule, a cDNA library made from a human hippocampus (Clontech, Palo Alto, Calif.) was screened under low stringency conditions as follows: Prehybridization for 4 hours at 42° C. in Clontech Express Hyb solution, followed by overnight hybridization at 42° C. The probe used was a PCR-generated fragment including nucletides 49-711 of the rat sequence labeled with 3²p dCTP. The filters were washed 6 times in 2× SSC/0.1% SDS at 55° C. The same conditions were used for secondary screening of the positive isolates. Clones thus obtained were sequenced using an ABI automated DNA Sequencing system, and compared to the rat sequences shown in SEQ ID NO:3 as well as to known sequences from the GenBank database. The largest clone from the library screen was subsequently subcloned into pBS-KS+ (Stratagene, La Jolla, Calif.) for sequence verification. The 515 base pair clone was determined to represent the human homolog of the 1v gene, encompasing 211 base pairs of 5′ UTR and a 304 base pair coding region. To generate the full-length cDNA, 3′ RACE was used according to the manufacturers instructions (Clontech Advantage PCR kit).

### Example 3

### Isolation and Characterization of 1V Splice Variants

The mouse 1v shown in SEQ ID NO:5 and the rat 1vl splice variant shown in SEQ ID NO:7 was isolated using a two-hybrid assay as described in Example 1. The mouse 1vl splice variant shown in SEQ ID NO:7 was isolated by screening a mouse brain cDNA library, and the rat 1vn splice variant shown in SEQ ID NO:11 was isolated by BLAST searching.

### Example 4

### Isolation and Identification of 9Q and Other PCIPs

Rat 9ql (SEQ ID NO:15) was isolated by database mining, rat 9qm (SEQ ID NO: 21) was isolated by a two-hybrid assay, and rat 9qc (SEQ ID NO:27) was identified by database mining. Human 9ql (SEQ ID NO: 13), and human 9qs (SEQ ID NO: 23) were identified as described in Example 2. Mouse 9ql (SEQ ID NO:17), monkey 9qs (SEQ ID NO:25), human p195 (SEQ ID NO:31), W28559 (SEQ ID NO:37), human p193 (SEQ ID NO:39), rat p19 (SEQ ID NO:33), and mouse p19 (SEQ ID NO:35) were identified by database mining. Rat 8t (SEQ ID NO:29) was identified using a two-hybrid assay.

The human genomic 9q sequence (SEQ ID NOs:46 and 47) was isolated by screening a BAC genomic DNA library (Reasearch Genetics) using primers which were designed based on the sequence of the human 9qm cDNA. Two positive clones were identified (44802 and 721117) and sequenced.

### Example 5

### Expression of p19, 1V, 8T, and 9Q mRNA in Rat Tissues

PCIP molecules, e.g., 9q and 8t, were demonstrated to be predominantly expressed in the heart. Briefly, rator mouse multiple tissue Northern blots (Clontech) were probed with a [³²P]-labeled cDNA probe directed at the p19 sequence, the 5′-untranslated and 5′-coding region of the rat 1v sequence (nucleotides 35-124; SEQ ID NO:3) (this probe is specific for rat 1v and rat 1vl), the 5′ coding region of the 8t sequence (nucleotides 1-88; SEQ ID NO:29) (this probe is specific for 8t), or the 5′ end of the rat 9qm sequence (nucleotides 1-195; SEQ ID NO:21) (this probe is specific for all 9q isoforms, besides 8t). Blots were hybridized using standard techniques.

The results indicated that p19 is expressed predominantly in the brain, but also in the heart. Moreover, northern blots hybridized with the rat 1v probe revealed a single band at 2.3 kb only in the lane containing brain RNA, suggesting that 1v expression is brain specific. Northern blots probed with the rat 8t probe revealed a major band at 2.4 kb. The rat 8t band was most intense in the lane containing heart RNA and there was also a weaker band in the lane containing brain RNA. Northern blots hybridized with the 9q cDNA probe revealed a major band at 2.5 kb and a minor band at over 4 kb with predominant expression in heart and brain. The minor band may represent incompletely spliced or processed 9q mRNA.

### Example 6

### Expression of 1V, 8T, and 9Q in Brain

Expression of the rat 1v and 8t/9q genes in the brain was examined by in situ hybridization histochemistry (ISHH) using [³⁵S]-labeled cRNA probes and a hybridization procedure identical to that described in Rhodes et al. (1996) J. Neurosci., 16:4846-4860. Templates for preparing the cRNA probes were generated by standard PCR methods. Briefly, oligonucleotide primers were designed to amplify a fragment of 3′- or 5′-untranslated region of the target cDNA and in addition, add the promoter recognition sequences for T7 and T3 polymerase. Thus, to generate a 300 nucleotide probe directed at the 3′-untranslated region of the 1v mRNA, we used the following primers:

5-TAATACGACTCACTATAGGGACTGGCCATCCTGCTCTCAG-3 (T7, forward, sense; SEQ ID NO:42)

5-ATTAACCCTCACTAAAGGGACACTACTGTTTAAGCTCAAG-3 (T3, reverse, antisense; SEQ ID NO:43). The underlined bases correspond to the T7 and T3 promoter sequences. To generate a probe directed at a 325 bp region of 3′-untranslated sequence shared by the 8t and 9q mRNAs, the following primers were used:

5-TAATACGACTCACTATAGGGCACCTCCCCTCCGGCTGTTC-3 (T7, forward, sense; SEQ ID NO:44)

5-ATTAACCCTCACTAAAGGGAGAGCAGCAGCATGGCAGGGT-3 (T3, reverse, antisense; SEQ ID NO:45).

Autoradiograms of rat brain tissue sections processed for ISHH localization of 1v or 8t/9q mRNA expression revealed that 1v mRNA is expressed widely in brain in a pattern consistent with labeling of neurons as opposed to glial or endothelial cells. 1v mRNA is highly expressed in cortical, hippocampal, and striatal interneurons, the reticlar nucleus of the thalamus, the medial habenula, and in cerebellar granule cells. 1v mRNA is expressed at moderate levels in midbrain nuclei including the substantia nigra and superior colliculus, in several other thalamic nuclei, and in the medial septal and diagonal band nuclei of the basal forebrain.

Because the probe used to analyze the expression of 8t and 9q hybridizes to a region of the 3-untranslated region that is identical in the 8t and 9q mRNAs, this probe generates a composite image that reveals that 8t/9q mRNA is expressed widely in brain in a pattern that partly overlaps with that for 1v as described above. However, 8t/9q mRNA is highly expressed in the striatum, hippocampal formation, cerebellar granule cells, and neocortex. 8t/9q mRNA is expressed at moderate levels in the midbrain, thalamus, and brainstem. In may of these areas, 8t./9q mRNA appears to be concentrated in interneurons in addition to principal cells, and in all regions 8t/9q expression appears to be concentrated in neurons as apposed to glial cells.

Single- and double-label immunohistochemistry revealed that the PCIP and Kv4 polypeptides are precisely colocalized in many of the cell types and brain regions where PCIP and Kv4 mRNAs are coexpressed. For example, 9qm colocalized with Kv4.2 in the somata and dendrites of hippocampal granule and pyramidal cells, neurons in the medial habenular nucleus and in cerebellar basket cells, while 1v colocalized with Kv4.3 in layer II neurons of posterior cingulate cortex, hippocampal interneurons, and in a subset of cerebellar granule cells. Immunoprecipitation analyses indicated that 1v and 9qm are coassociated with Kv4 α-subunits in rat brain membranes.

### Example 7

### Co-association OF 1V AND Kv4.3 in COS Cells

COS1 cells were transiently transfected with rat Kv4.3 alone, rat Kv4.3+rat 1v, and rat 1v alone using the lipofectamine plus procedure essentially as described by the manufacturer (Boehringer Mannheim). Forty-eight hours after the transfection, cells were washed, fixed, and processed for immunofluorescent visualization as described previously (Bekele-Arcuri et al. (1996) Neuropharmacology, 35:851-865). Affinity-purified rabbit polyclonal or mouse monoclonal antibodies to the Kv4.3 or rat 1v protein were used for immunofluorescent detection of the target proteins. Cells transfected with 1v alone and stained with 1v-specific revealed that 1v is diffusely distributed throughout the cytoplasm of transiently transfected cells, as expected for a cytoplasmic protein. Cells transiently transfected with Kv4.3 alone and stained with antibodies specific for Kv4.3 revealed that although much of the expressed channel protein is trapped within intracellular organelles, Kv4.3 expression is also concentrated at the outer margins of the cell and is presumed to be associated with the cell membrane. When the 1v protein is coexpressed with Kv4.3 in COS1 cells, the subcellular distribution of 1v is dramatically different than it is in cells transfected with 1v alone. In cells cotransfected with 1v and Kv4.3, 1v protein expression appears to be trapped in intracellular organelles and becomes concentrated at the outer margins of the cell. Double-label immunofluorescence of these co-transfected cells indicates that the pattern of 1v immunofluorecence is identical that for Kv4.3, indicating that these two proteins are extensively colocalized in cotransfected cells. Moreover, the extensive and dramatic change in the subcellular distribution of 1v when it is coexpressed with Kv4.3 suggests that the proteins coassociate when they are coexpressed.

To further demonstrate that 1v and Kv4.3 directly associate in cotransfected cells, COS1 cells were cotransfected with 1v and Kv4.3 cDNAs as described above. The cells were then lysed in buffer containing detergent and protease inhibitors, and prepared for immunoprecipitation reactions essentially as described previously (Nakahira et al. (1996) J. Biol. Chem., 271:7084-7089). Antibodies specific for 1v or Kv4.3 were used to immunoprecipitate the corresponding polypeptide from the transfected cell lysates essentially as described in Nakahira et al. (1996) J. Biol. Chem., 271:7084-7089 and in Harlow E. and Lane, D., Antibodies: A Laboratory Manual, Cold Spring Harbor Laboratory, c1988. The products resulting from the immunoprecipitation were size fractionated by SDS-PAGE and transferred to nitrocellulose filters using standard procedures. Immunoblots performed using Kv4.3-specific antibodies revealed that 1v co-immunoprecipitates Kv4.3 from lysates prepared from co-transfected cells, indicating that the two proteins are tightly co-associated. Taken together, these data suggest that 1v may promote the transit of the Kv4.3 subunits to the cell surface, and that this chaperone-like effect may underlie the enhancement of Kv4 current density by 1v.

### Example 8

### Electrophysiological Characterization of PCIPs

Currents flowing through rKv4.2 were measured electrophysiologically after transiently transfecting the channels (with or without rat 1v) in CHO cells or microinjecting in vitro transcribed mRNA into Xenopus oocytes. Currents in CHO cells were measured using the patch-clamp technique (Hamill et al. 1981. Pfluegers Arch. 391:85-100), while currents in oocytes were measured with two-electrode voltage clamp. CHO cells were transiently-transfected with cDNA using the DOTAP lipofection method as described by the manufacturer. (Boehringer Mannheim, Inc.). Transfected cells were identified by cotransfecting enhanced GFP along with the genes of interest and subsequently determining if the cells contained green GFP fluorescence. Alternatively, oocytes were injected with 1-3 ng/oocyte cRNA which was prepared using standard in vitro transcription techniques (Sambrook et al. 1989. Molecular Cloning: a laboratory manual, Cold Spring Harbor Press). When CHO cells were transfected with 1 μg rKv4.2 cDNA, current levels averaged about 539 pA/cell, or 23.1 pA/pF (Table 2). When 1v was coexpressed with rKv4.2, however, the current amplitude increased by 8.5 fold to an average of 3076 pA/cell or 197.2 pA/pF (see below).

TABLE 2

| | CHO | | Oocytes | | Oocytes | |
|---|---|---|---|---|---|---|
| | | rKv4.2 | | hKv4.3 | | hKv1.4 |
| Parameter | rKv4.2 | + 1v | hKv4.3 | + 1v | hKv1.4 | + 1v |
| Peak Current | 538.8 | 3076.3 | 7.7 A | 18.1 A | 8.3 A | 6.5 A |
| (pA/cell) | | | | | | |
| Peak Current | 3.1 | 197.2 | — | — | — | — |
| (pA/pF) | | | | | | |
| Inactivation | 0.4 | 90.9 | 58.5 | 137.0 | 52.3 | 57.8 |
| time constant | | | | | | |
| (ms, at 40 mV) | | | | | | |
| Recovery from | 47.3 | 39.7 | 327.0 | 34.5 | 132.6, | 210.7, |
| Inactivation | | | | | | |
| time constant | | | | | | |
| (ms, | | | | | 666.8 | 821.9 |
| at −80 mV) | | | | | | |
| Activation | 13.1 | −15.9 | −19.2 | −45.5 | −21.0 | −13.5 |
| V_{1/2}(mV) | | | | | | |
| Steady-state | −54.1 | −59.7 | −57.4 | −56.8 | −47.5 | −48.1 |
| Inactivation | | | | | | |
| V_{1/2}(mV) | | | | | | |

Coexpression of 1v also caused a number of changes in other kinetic parameters of the rKv4.2 current. The voltage at which half of the channels are activated is a measure of the voltage dependence of the channels. This half activation voltage for rKv4.2 was relatively high at 13 mV. Coexpression of 1v with rKv4.2 shifted the half activation voltage by 29 mV to the more negative potential of -16 mV (Table 2). The voltage at which channels inactivate during a long (1 second) pulse only shifted slightly from −54 to -60 mV with 1v coexpression.

The modulatory effects observed with 1v coexpression were not limited to the rKv4.2 channel or to CHO cells. A similar modulation by 1v of hKv4.3 expressed in Xenopus oocytes has also been observed (see Table 2). Co-injection of 1v into oocytes induced an increase in hKv4.3 current, a slowing of inactivation, a speeding of the recovery from inactivation, and a leftward shift in the activation curve. The effects of 1v, however, did not translate to all inactivating channel types, as the inactivating hKv1.4 channel was not effected by coinjection of 1v mRNA into oocytes (Table 2).

Co-expression of 1v or 9qm with Kv4 α-subunits in CHO cells or Xenopus oocytes revealed that the corresponding polypeptides co-associate with Kv4 subunits and dramatically modulate the current density, rate of inactivation and rate of recovery from inactivation of Kv4 channels.

Deletion of the N-terminus of the two PCIP proteins 1v and 9qm (the first 31 amino acids were deleted from 1v and the first 67 amino acids were deleted from 9qm) did not alter their modulatory actions on Kv4.2 current amplitude and kinetics when co-expressed in CHO cells. Thus, the variable N-terminus of these genes is not responsible for their modulatory actions on Kv4 channels. Point mutations were then constructed in the EF-hand domains of the 1v gene to remove its putative ability to bind calcium. Two different mutants were created: one has point mutations in the first two EF hands (D₁₉₉ to A, G₁₀₄ to A, D₁₃₅ to A, and G₁₄₀ to A) and the other one has point muations in all three EF hands (D₁₉₉ to A, G₁₀₄ to A, D₁₃₅ to A, G₁₄₀ to A, D₁₈₃ to A, and G₁₈₈ to A). These mutations had a large effect on the modulatory function of this gene; co-expression with Kv4.2 produced a much smaller increase in current than the wild type 1v and very little effect on the other kinetic parameters of the channel. Thus, the EF-hand, or putative Ca²⁺ binding domains, of 1v appear to have a critical role in the modulatory actions of the PCIP genes.

### Example 9

### Characterization of the PCIP Proteins

In this example, the amino acid sequences of the PCIP proteins were compared to amino acid sequences of known proteins and various motifs were identified.

The 1v polypeptide, the amino acid sequence of which is shown in SEQ ID NO:3 is a novel polypeptide which includes 216 amino acid residues. Domains that are putatively involved in calcium binding (Linse, S. and Forsen, S. (1995) Advances in Second Messenger and Phosphoprotein Research 30, Chapter 3, p89-151, edited by Means, A R., Raven Press, Ltd., New York), were identified by sequence alignment (see FIG. 21).

The 8t polypeptide, the amino acid sequence of which is shown in SEQ ID NO:30 is a novel polypeptide which includes 225 amino acid residues. Calcium binding domains that are putatively involved in calcium binding (Linse, S. and Forsen, S. (1995) Advances in Second Messenger and Phosphoprotein Research 30, Chapter 3, p89-151, edited by Means, A R., Raven Press, Ltd., New York), were identified by sequence alignment (see FIG. 21).

The 9q polypeptide is a novel polypeptide which includes calcium binding domains that are putatively involved in calcium binding (Linse, S. and Forsen, S. (1995) Advances in Second Messenger and Phosphoprotein Research 30, Chapter 3, p89-151, edited by Means, A R., Raven Press, Ltd., New York (see FIG. 21).

The p19 polypeptide is a novel polypeptide which includes calcium binding domains that are putatively involved in calcium binding (Linse, S. and Forsen, S. (1995) Advances in Second Messenger and Phosphoprotein Research 30, Chapter 3, p89-151, edited by Means, A R., Raven Press, Ltd., New York (see FIG. 21).

A BLASTN 2.0.7 search (Altschul et al. (1990) J. Mol. Biol. 215:403) of the nucleotide sequence of rat 1vl revealed that the rat 1vl is similar to the rat cDNA clone RMUAH89 (Accession Number AA849706). The rat 1 vl nucleic acid molecule is 98% identical to the rat cDNA clone RMUAH89 (Accession Number AA849706) over nucleotides 1063 to 1488.

A BLASTN 2.0.7 search (Altschul et al. (1990) J. Mol. Biol. 215:403) of the nucleotide sequence of human 9ql revealed that the human 9ql is similar to the human cDNA clone 1309405 (Accession Number AA757119). The human 9 ql nucleic acid molecule is 98% identical to the human cDNA clone 1309405 (Accession Number AA757119) over nucleotides 937 to 1405.

A BLASTN 2.0.7 search (Altschul et al. (1990) J. Mol. Biol. 215:403) of the nucleotide sequence of mouse P19 revealed that the mouse P19 is similar to the Mus musculus cDNA clone MNCb-7005 (Accession Number AU035979). The mouse P19 nucleic acid molecule is 98% identical to the Mus musculus cDNA clone MNCb-7005 (Accession Number AU035979) over nucleotides 1 to 583.

### Example 10

### Expression of Recombinant PCIP Proteins in Bacterial Cells

In this example, PCIP is expressed as a recombinant glutathione-S-transferase (GST) fusion polypeptide in E. coli and the fusion polypeptide is isolated and characterized. Specifically, PCIP is fused to GST and this fusion polypeptide is expressed in E. coli, e.g., strain BI21. Expression of the GST-PCIP fusion protein in BI21 is induced with IPTG. The recombinant fusion polypeptide is purified from crude bacterial lysates of the induced BI21 strain by affinity chromatography on glutathione beads. Using polyacrylamide gel electrophoretic analysis of the polypeptide purified from the bacterial lysates, the molecular weight of the resultant fusion polypeptide is determined.

Rat 1v and 9ql were cloned into pGEX-6p-2 (Pharmacia). The resulting recombinant fusion proteins were expressed in E. coli cells and purified following art known methods (described in, for example, Current Protocols in Molecular Biology, eds. Ausubel et al. John Wiley & Sons: 1992). The identities of the purified proteins were verified by western blot analysis using antibodies raised against peptide epitopes of rat 1v and 9ql.

### Example 11

### Expression of Recombinant PCIP Proteins in COS Cells

To express the PCIP gene in COS cells, the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire PCIP protein and an HA tag (Wilson et al. (1984) Cell 37:767) or a FLAG tag fused in-frame to its 3′ end of the fragment is cloned into the polylinker region of the vector, thereby placing the expression of the recombinant protein under the control of the CMV promoter.

To construct the plasmid, the PCIP DNA sequence is amplified by PCR using two primers. The 5′ primer contains the restriction site of interest followed by approximately twenty nucleotides of the PCIP coding sequence starting from the initiation codon; the 3′ end sequence contains complementary sequences to the other restriction site of interest, a translation stop codon, the HA tag or FLAG tag and the last 20 nucleotides of the PCIP coding sequence. The PCR amplified fragment and the pCDNA/Amp vector are digested with the appropriate restriction enzymes and the vector is dephosphorylated using the CIAP enzyme (New England Biolabs, Beverly, Mass.). Preferably the two restriction sites chosen are different so that the PCIP gene is inserted in the correct orientation. The ligation mixture is transformed into E. coli cells (strains HB101, DH5a, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the PCIP-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989. The expression of the PCIP polypeptide is detected by radiolabelling (³⁵S-methionine or ³⁵S-cysteine available from NEN, Boston, Mass., can be used) and immunoprecipitation (Harlow, E. and Lane, D. Antibodies: A Laboratory Manual, Cold Spring Harbor Laboratory Press, Cold Spring Harbor, N.Y., 1988) using an HA specific monoclonal antibody. Briefly, the cells are labelled for 8 hours with ³⁵S-methionine (or ³⁵S-cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA specific monoclonal antibody. Precipitated polypeptides are then analyzed by SDS-PAGE.

Alternatively, DNA containing the PCIP coding sequence is cloned directly into the polylinker of the pCDNA/Amp vector using the appropriate restriction sites. The resulting plasmid is transfected into COS cells in the manner described above, and the expression of the PCIP polypeptide is detected by radiolabelling and immunoprecipitation using a PCIP specific monoclonal antibody.

Rat 1v was cloned into the mammalian expresssion vector pRBG4. Transfections into COS cells were performed using LipofectAmine Plus (Gibco BRL) following the manufacturer's instructions. The expressed 1v protein was detected by immunocytochemistry and/or western blot analysis using antibodies raised against 1v in rabbits or mice.

### Example 12

### Identification and Characterization of Human Full Length p19

The human full length p19 sequence was identified using RACE PCR. The sequence of p19 (also referred to as KChIP3) is shown in FIG. 16. The amino acid sequence of human p19 is 92% identical to the mouse p19 gene (SEQ ID NO:35).

TBLASTN searches using the protein sequence of human p19 revealed that human p19 is homologous to two sequences, Calsenilin (described in (1998) Nature Medicine 4: 1177-1181) and DREAM (described in Carrion et al. (1999) Nature 398:80-84). Human p19 is 100% identical at the nucleotide level to Calsenilin (but extends 3′ to the published sequence) and 99% identical at the nucleotide level to DREAM.

The ability of p19 (as well as other PCIP family members) to co-localize with presenilin and act as transcription factors is determined using art known techniques such as northern blots, in situ hybridization, β-gal assays, DNA mobility assays (described in, for example, Carrion et al. (1999) Nature 398:80) and DNA mobility supershift assays, using antibodies specific for KchIPs.

Other assays suitable for evaluating the association of PCIP family members with presenilins is co-immunoprecipitation (described in, for example, Buxbaum et al. (1998) Nature Medicine 4:1177).

### Example 13

### Identification and Characterization of Monkey KChIP4

In this example, the identification and characterization of the genes encoding monkey KChIP4a (jlkbd352e01t1) and alternatively spliced monkey KChIP4b (jlkbb231c04t1), KChIP4c (jlkxa053c02), and KChIP4d (jlkx015b10) is described. TBLASTN searches in proprietary databases with the sequence of the known PCIP family members, lead to the identification of four clones jlkbb231c04t1, jlkbd352e01t1, jlkxa053c02, and jlkx015b10. The four monkey clones were obtained and sequenced.

The sequences of proprietary monkey clones jlkbb231c04t1 and jlkbd352e01t1 were found to correspond to alternately spliced variants of an additional PCIP family member, referred to herein as KChIP4. Clone jlkbb231c04t1 contains a 822 bp deletion relative to jlkbd352e01t1 presumably due to splicing out of an exon), resulting in the loss of the final EF hand domain. In clone jlkbd352e01t1, the final EF hand domain is preserved, and the C-terminus is highly homologous to that of PCIP family members 1v, 9ql, and p19. Overall identity in the homologous C-termini among KChIP4, 1v, 9ql, and p19 ranged from 71%-80% at the amino acid level (alignments were performed using the CLUSTALW).

Monkey KChIP4c and KChIP4d were discovered by BLASTN search using monkey KChIP4a as a query for searching a proprietary database.

The nucleotide sequence of the monkey KChIP4a cDNA and the predicted amino acid sequence of the KChIP4a polypeptide are shown in FIG. 23 and in SEQ ID NOs:48 and 49, respectively.

The nucleotide sequence of the monkey KChIP4b cDNA and the predicted amino acid sequence of the KChIP4b polypeptide are shown in FIG. 24 and in SEQ ID NOs:50 and 51, respectively.

The nucleotide sequence of the monkey KChIP4c cDNA and the predicted amino acid sequence of the KChIP4c polypeptide are shown in FIG. 35 and in SEQ ID NOs:69 and 70, respectively.

The nucleotide sequence of the monkey KChIP4d cDNA and the predicted amino acid sequence of the KChIP4d polypeptide are shown in FIG. 36 and in SEQ ID NOs:71 and 72, respectively.

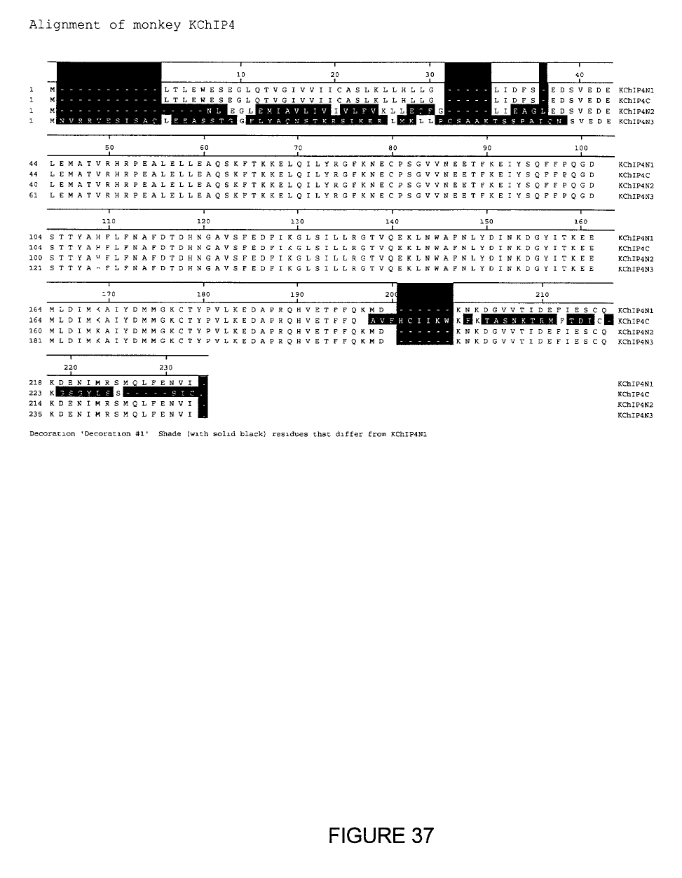

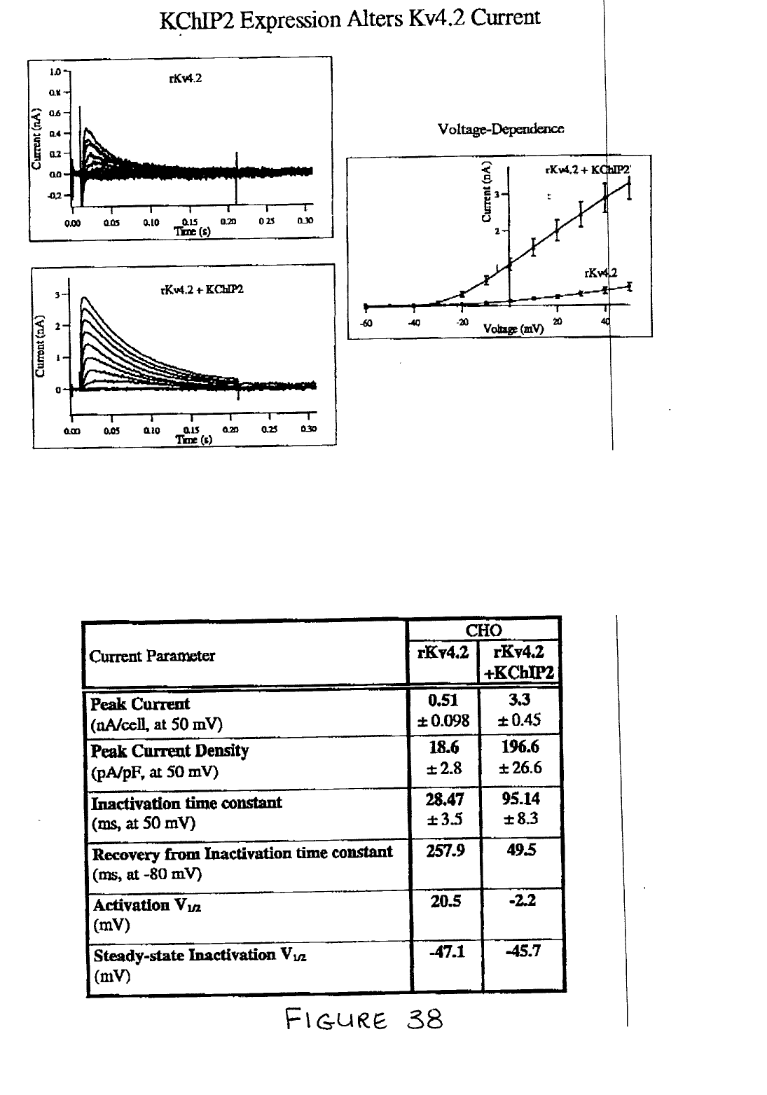

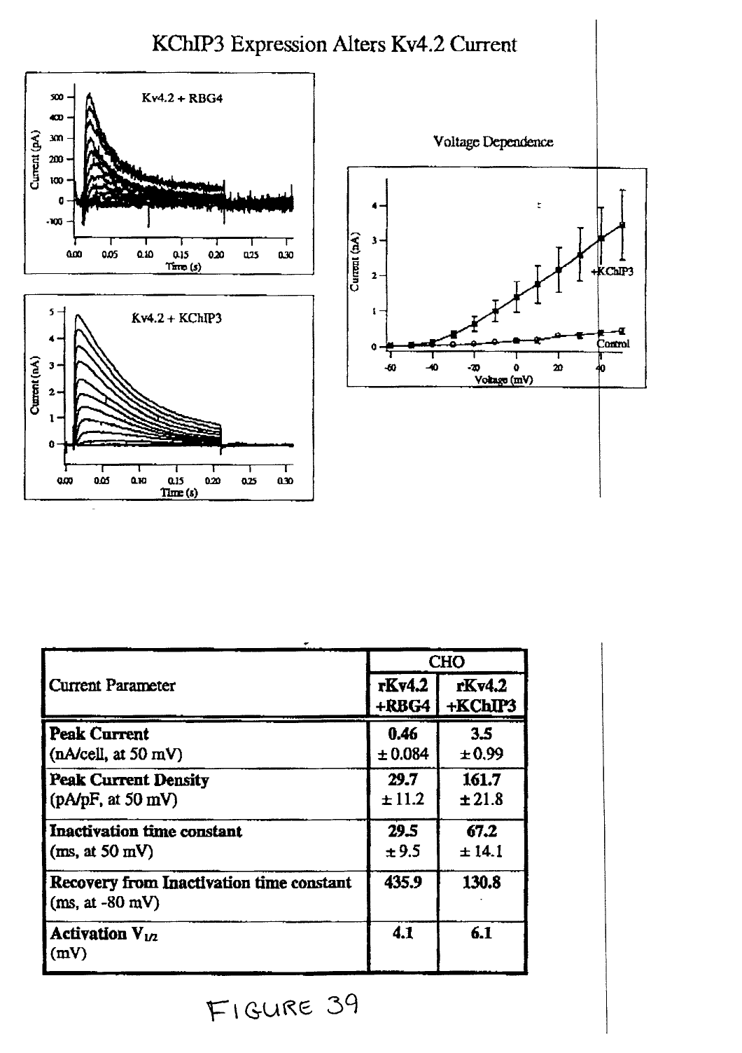

FIG. 37 depicts an alignment of the protein sequences of KChIP4a, KChIP4b, KChIP4c, and KChIP4d.

### Example 14

### Identification and Characterization of Human and Rat 33b07

In this example, the identification and characterization of the genes encoding rat and human 33b07 is described. Partial rat 33b07 (clone name 9o) was isolated as a positive clone from the yeast two-hybrid screen described above, using rKv4.3N as bait. The full length rat 33b07 clone was identified by mining of proprietary databases.

The nucleotide sequence of the full length rat 33b07 cDNA and the predicted amino acid sequence of the rat 33b07 polypeptide are shown in FIG. 26 and in SEQ ID NOs:52 and 53, respectively. The rat 33b07 cDNA encodes a protein having a molecular weight of approximately 44.7 kD and which is 407 amino acid residues in length.

Rat 33b07 binds rKv4.3N and rKv4.2N with slight preference for rKv4.2N in yeast 2-hybrid assays. In contrast, rat 33b07 does not bind rKv1.1N, indicating that the rat 33b07-Kv4N interaction is specific.

Rat 33b07 is expressed predominantly in the brain as determined by northern blot analysis.

The human 33b07 ortholog (clone 106d5) was also identified by mining of proprietary databases. The nucleotide sequence of the full length human 33b07 cDNA and the predicted amino acid sequence of the human 33b07 polypeptide are shown in FIG. 27 and in SEQ ID NOs:54 and 55, respectively. The human 33b07 cDNA encodes a protein having a molecular weight of approximately 45.1 kD and which is 414 amino acid residues in length.

Human 33b07 is 99% identical to the human KIAA0721 protein (GenBank Accession Number: ABOL 8264) at the amino acid level. However, GenBank Accession Number: AB018264 does not have a functional annotation. Human 33b07 is also homologous to Testes-specific (Y-encoded) proteins (TSP(Y)s), SET, and Nucleosome Assembly Proteins (NAPs). The human 33b07 is 38% identical to human SET protein (GenBank Accession Number Q01105=U51924) over amino acids 204 to 337 and 46% identical over amino acids 334 to 387.

Human SET is also called HLA-DR associated protein II (PHAPII) (Hoppe-Seyler (1994) Biol. Chem. 375:113-126) and in some cases is associated with acute undifferentiated leukemia (AUL) as a result of a translocation event resulting in the formation of a SET-CAN fusion gene (Von Lindern M. et al. (1992) Mol. Cell. Biol. 12:3346-3355). An alternative spliced form of SET is also called Template Activating Factor-I alpha (TAF). TAF is found to be associated with myeloid leukemogenesis (Nagata K. et al. (1995) Proc. Natl. Acad. Sci. U.S.A. 92 (10), 4279-4283). Human SET is also a potent protein inhibitor of phosphatase 2A (Adachi Y. et al. (1994) J. Biol. Chem. 269:2258-2262). NAPs may be involved in modulating chromatin formation and contribute to regulation of cell proliferation (Simon H. U. et al. (1994) Biochem. J. 297, 389-397).

Thus, due to its homology to the above identified proteins, 33b07 may function as a protein inhibitor of phosphatase, an oncogene, and/or a chromatin modulator. The homology of 33b07 to SET, a protein phosphatase inhibitor, is of particular interest. Many channels, in particular the Kv4 channels (with which 33b07 is associated), are known to be regulated by phosphorylation by PKC and PKA ((1998) J Neuroscience 18(10):3521-3528; Am J Physiol 273: H1775-86 (1997)). Thus, 33b07 may modulate Kv4 activity by regulating the phosphorylation status of the potassium channel.

### Example 15

### Identification and Characterization of Rat 1p

In this example, the identification and characterization of the gene encoding rat 1p is described. Partial rat 1p was isolated as a positive clone from the yeast two-hybrid screen described above, using rKv4.3N as a bait.

The nucleotide sequence of the partial length rat 1p cDNA and the predicted amino acid sequence of the rat 1p polypeptide are shown in FIG. 28 and in SEQ ID NOs:56 and 57, respectively. The rat 1p cDNA encodes a protein having a molecular weight of approximately 28.6 kD and which is 267 amino acid residues in length.

Rat 1p binds rKv4.3N and rKv4.2N with slight preference for rKv4.3N in yeast two-hybrid assays. In contrast, 1p does not bind rKv1.1N, indicating that the 11p-Kv4N interaction is specific.

Rat 1p is predominantly expressed in the brain as determined by northern blot analysis.

A BLASTP 1.4 search, using a score of 100 and a word length of 3 (Altschul et al. (1990) J. Mol. Biol. 215:403) of the amino acid sequences of rat 1p revealed that rat 1p is similar to the human Restin (GenBank Accession Number P30622; also named cytoplasmic linker protein-170 alpha-2 (CLIP-170), M97501)). The rat 1p protein is 58% identical to the human Restin over amino acid residues 105 to 182, 55% identical to the human Restin over amino acid residues 115 to 186, 22% identical to the human Restin over amino acid residues 173 to 246, 22% identical to the human Restin over amino acid residues 169 to 218, and 58% identical to the human Restin over amino acid residues 217 to 228.

Restin is also named Reed-Sternberg intermediate filament associated protein. Reed-Sternberg cells are the tumoral cells diagnostic for Hodgkin's disease. It is suggested that Restin overexpression may be a contributing factor in the progression of Hodgkin's disease (Bilbe G. et al. (1992) EMBO J. 11:2103-13) and Restin appears to be an intermediate filament associated protein that links endocytic vesicles to microtubules (Pierre P, et al. (1992) Cell 70 (6), 887-900).

The cytoskeleton regulates the activity of potassium channels (see, for example, Honore E, et al. (1992) EMBO J. 11:2465-2471 and Levin G, et al. (1996) J. Biol. Chem. 271:29321-29328), as well as the activity of other channels, e.g., Ca⁺⁺ channels (Johnson B. D. et al. (1993) Neuron 10:797-804); or Na⁺ channels (Fukuda J. et al. (1981) Nature 294:82-85).

Accordingly, based on its homology to the Restin protein, the rat 1p protein may be associated with the cytoskeleton and may modulate the activity of potassium channels, e.g., Kv4, via its association to the cytoskeleton.

### Example 16

### Identification and Characterization of Rat 7s

In this example, the identification and characterization of the gene encoding rat 7s is described. Partial rat 7s was isolated as a positive clone from the yeast two-hybrid screen described above, using rKv4.3N as a bait. Rat 7s is the rat ortholog of the human vacuolar H(+)-ATPase catalytic subunit A (Accession Number P38606 and B46091) described in, for example, van Hille B. et al. (1993) J. Biol. Chem. 268 (10), 7075-7080.

The nucleotide sequence of the partial length rat 7s cDNA and the predicted amino acid sequence of the rat 7s polypeptide are shown in FIG. 29 and in SEQ ID NOs:58 and 59, respectively. The rat 7s cDNA encodes a protein having a molecular weight of approximately 28.6 kD and which is 270 amino acid residues in length.

Rat 7s binds rKv4.3N and rKv4.2N with preference for rKv4.3N in yeast two-hybrid assays. In contrast, 7s does not bind rKv1.1N, indicating that the 7s-Kv4N interaction is specific.

Rat 7s is expressed at significantly higher levels in the brain and the kidney than in the lung, liver, heart, testes, and skeletal muscle, as determined by northern blot analysis.

### Example 17

### Identification and Characterization of Rat 29x and 25r

In this example, the identification and characterization of the gene encoding rat 29x is described. Rat 29x was isolated as a positive clone from the yeast two-hybrid screen described above, using rKv4.3N as a bait. Rat 25r is a splice variant of 29x. They differ in the 5′ untranslated region, but are identical in the coding region and at the amino acid level.

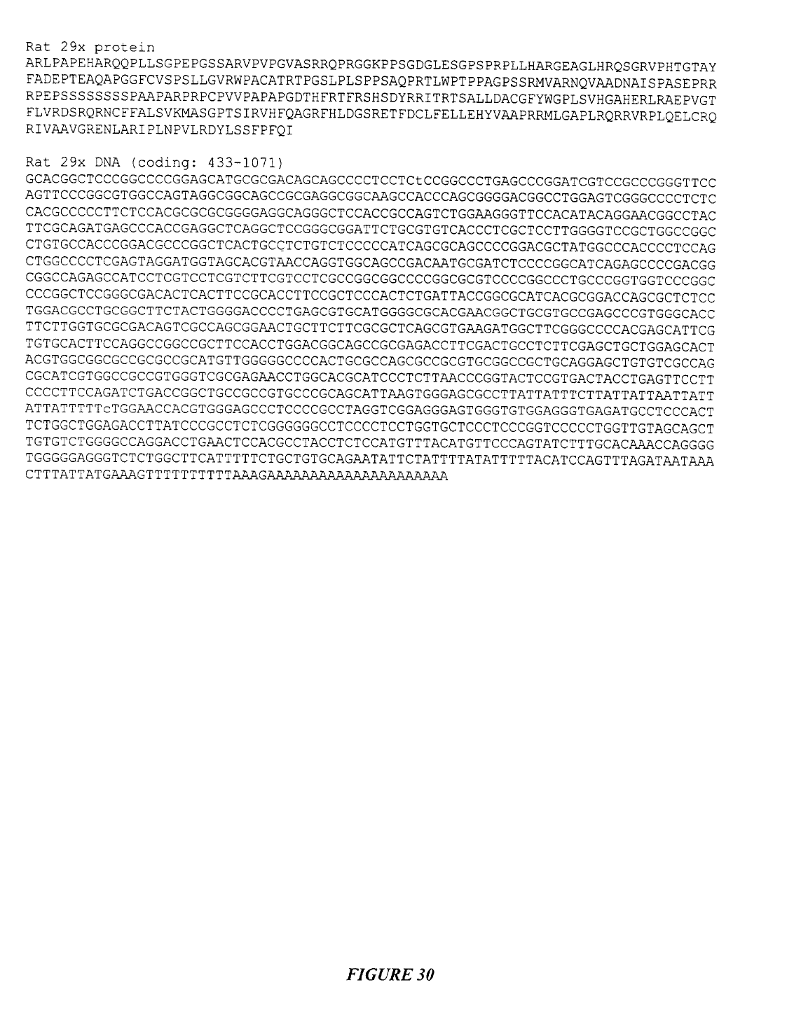

The nucleotide sequence of the rat 29x cDNA and the predicted amino acid sequence of the rat 29x polypeptide are shown in FIG. 30 and in SEQ ID NOs:60 and 61, respectively. The rat 29x cDNA encodes a protein having a molecular weight of approximately 40.4 kD and which is 351 amino acid residues in length.

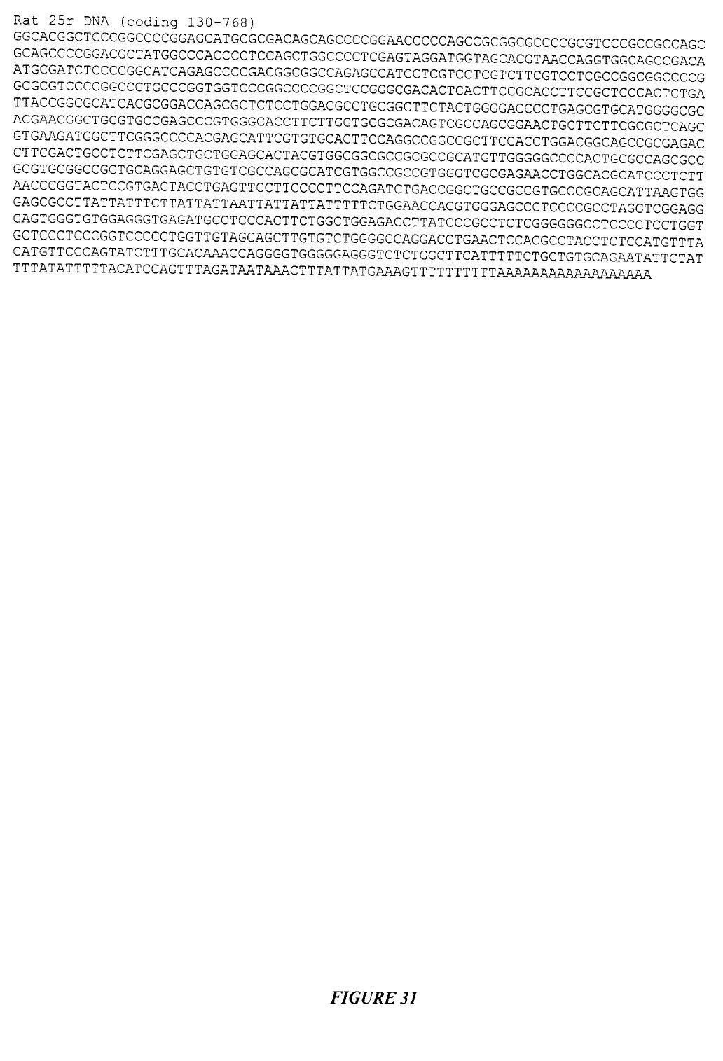

The nucleotide sequence of the rat 25r cDNA is shown in FIG. 31 and in SEQ ID NO:62. The rat 25r cDNA encodes a protein having a molecular weight of approximately 40.4 kD and which is 351 amino acid residues in length.

Rat 29x is expressed in the spleen, lung, kidney, heart, brain, testes, skeletal muscle and liver, with the highest level of expression being in the spleen and the lowest being in the liver.

Rat 29x binds rKv4.3N and rKv4.2N with slight preference for rKv4.3N in yeast two-hybrid assays. In contrast, 29x does not bind rKv1.1N, indicating that the 29x-Kv4N interaction is specific.

Rat 29x is identical at the amino acid level to rat SOCS-1 (Suppressor Of Cytokine Signaling) described in Starr R. et al. (1997) Nature 387:917-921; to JAB described in Endo T. A. et al. (1997) Nature 387:921-924; and to SSI-1 (STAT-induced STAT inhibitor-1) described in Naka T. et al. (1997) Nature 387:924-928. These proteins are characterized in that they have an SH2 domain, bind to and inhibit JAK kinase, and, as a result, regulate cytokine signaling.

As used herein, the term “SH2 domain”, also referred to a Src Homology 2 domain, includes a protein domain of about 100 amino acids in length which is involved in binding of phosphotyrosine residues, e.g., phosphotyrosine residues in other proteins. The target site is called an SH2-binding site. The SH2 domain has a conserved 3D structure consisting of two alpha helices and six to seven beta-strands. The core of the SH2 domain is formed by a continuous beta-meander composed of two connected beta-sheets (Kuriyan J. et al. (1997) Curr. Opin. Struct. Biol. 3:828-837). SH2 domains function as regulatory modules of intracellular signaling cascades by interacting with high affinity to phosphotyrosine-containing target peptides in a sequence-specific and strictly phosphorylation-dependent manner (Pawson T. (1995) Nature 373:573-580). Some proteins contain multiple SH2 domains, which increases their affinity for binding to phosphoproteins or confers the ability to bind to different phosphoproteins. Rat 29x contains an SH2 domain at amino acid residues 219-308 of SEQ ID NO:61.

Tyrosine phosphorylation regulates potassium channel activity (Prevarskaya N.B. et al. (1995) J. Biol. Chem. 270:24292-24299). JAK kinase phoshorylates proteins at tyrosines and is implicated in the regulation of channel activity (Prevarskaya N.B. et al. supra). Accordingly, based on its homology to SOCS-1, JAB, and SSI-1, rat 29x may modulate the activity of potassium channels, e.g., Kv4, by modulating JAK kinase activity.

### Example 18

### Identification and Characterization of Rat 5p

In this example, the identification and characterization of the gene encoding rat 5p is described. Rat 5p was isolated as a positive clone from the yeast two-hybrid screen described above, using rKv4.3N as a bait.

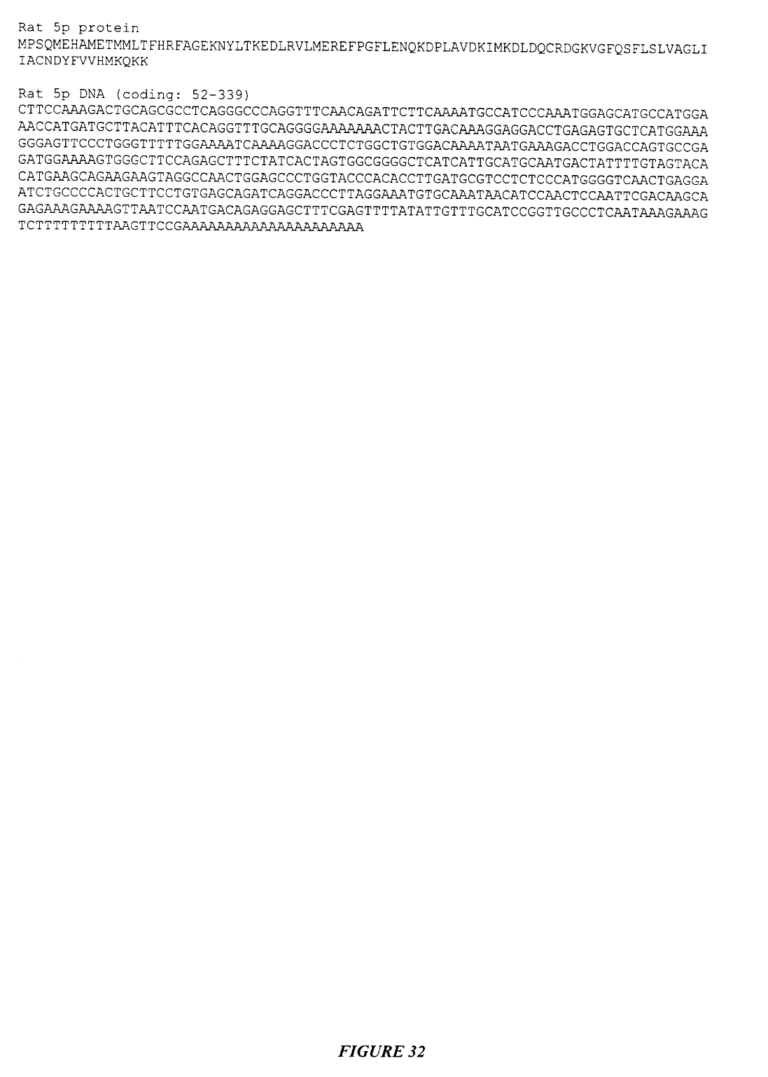

The nucleotide sequence of the rat 5pc DNA and the predicted amino acid sequence of the rat 5p polypeptide are shown in FIG. 32 and in SEQ ID NOs:63 and 64, respectively. The rat 5p cDNA encodes a protein having a molecular weight of approximately 11.1 kD and which is 95 amino acid residues in length.

Rat 5p binds rKv4.3N and rKv4.2N with similar strength in yeast two-hybrid assays. In contrast, 5p does not bind rKv1.1N, indicating that the 5p-Kv4N interaction is specific.

Rat 5p is expressed in the spleen, lung, skeletal muscle, heart, kidney, brain, liver, and testes, as determined by northern blot analysis.

The rat 5p is identical to rat Calpactin I light chain or P10 (Accession Number P05943). P10 binds and induces the dimerization of annexin II (p36). P10 may function as a regulator of protein phosphorylation in that the p36 monomer is the preferred target of a tyrosine-specific kinase (Masiakowski P. et al. (1998) Proc. Natl. Acad. Sci. U.S.A. 85 (4): 1277-1281).

Tyrosine phosphorylation regulates the activity of potassium channels (Prevarskaya N. B. et al. supra). Thus, due to its identity to P10, rat 5p may modulate the activity of potassium channels, e.g., Kv4, by modulating the activity of a tyrosine-specific kinase.

### Example 19

### Identification and Characterization of Rat 7q

In this example, the identification and characterization of the gene encoding rat 7q is described. Rat 7q was isolated as a positive clone from the yeast two-hybrid screen described above, using rKv4.3N as a bait. Full length rat 7q was obtained by RACE PCR.

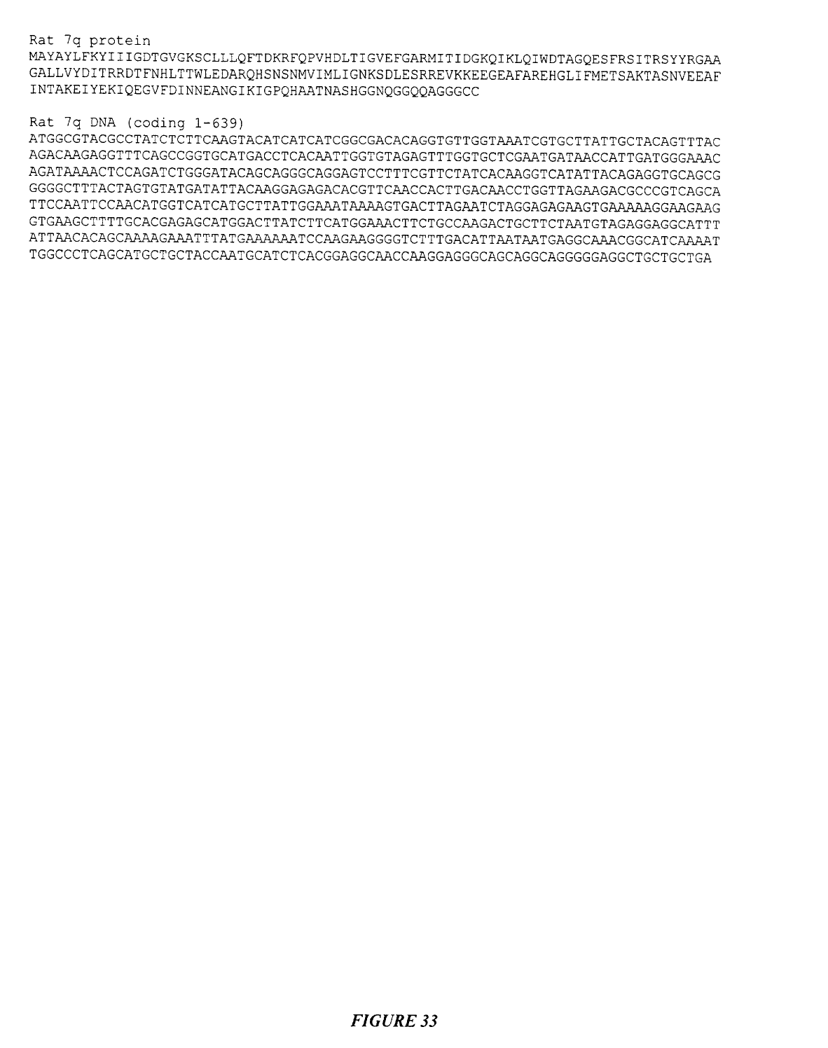

The nucleotide sequence of the rat 7q cDNA and the predicted amino acid sequence of the rat 7q polypeptide are shown in FIG. 33 and in SEQ ID NOs:65 and 66, respectively. The rat 7q cDNA encodes a protein having a molecular weight of approximately 23.5 kD and which is 212 amino acid residues in length.

Rat 7q binds rKv4.3N and rKv4.2N with same strength in yeast two-hybrid assays. In contrast, 7q does not bind rKv1.1N, indicating that the 7q-Kv4N interaction is specific.

Rat 7q is expressed in the heart, brain, spleen, lung, liver, skeletal muscle, kidney, and testes, as determined by northern blot analysis.

Rat 7q is identical to RAB2 (rat RAS-related protein, Accession Number P05712) at the amino acid level. RAB2 appears to be involved in vesicular traffic and protein transport (Touchot N. et al. (1987) Proc. Natl. Acad. Sci. U.S.A. 84 (23):8210-8214). Accordingly, based on its homology to RAB2, rat 7q may be involved in potassium channel, e.g., Kv4, trafficking.

### Example 20

### Identification and Characterization of Rat 19r

In this example, the identification and characterization of the gene encoding rat 19r is described. Partial rat 19r was isolated as a positive clone from the yeast two-hybrid screen described above, using rKv4.3N as a bait. Full length rat 19r was obtained by RACE PCR.

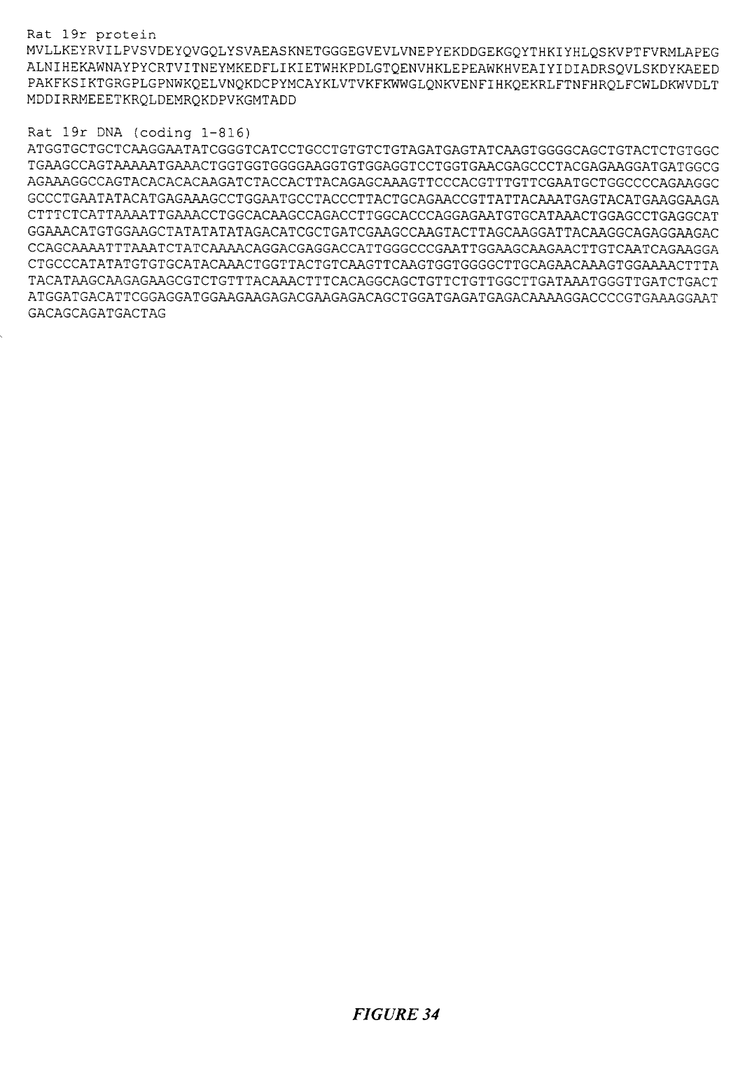

The nucleotide sequence of the rat 19r cDNA and the predicted amino acid sequence of the rat 19r polypeptide are shown in FIG. 34 and in SEQ ID NOs:67 and 68, respectively. The rat 19r cDNA encodes a protein having a molecular weight of approximately 31.9 kD and which is 271 amino acid residues in length.

Rat 19r is expressed in the heart, brain, spleen, lung, liver, skeletal muscle, kidney, and testes, as determined by northern blot analysis.

Rat 19r binds rKv4.3N and rKv4.2N with slight preference for rKv4.3N in yeast two-hybrid assays. In contrast, 19r does not bind rKv1.1N, indicating that the 19r-Kv4N interaction is specific.

Rat 19r is identical to Rat phosphatidylinositol (PTDINS) transfer protein alpha (PTDINSTP, Accession Number M25758 or P16446) described in Dickeson S. K. et al. (1989) J. Biol. Chem. 264:16557-16564. PTDINSTP is believed to be involved in phospholipase C-beta (PLC-beta) signaling, phosphatidylinositol transfer protein (PtdIns-TP) synthesis, secrettory vesicle formation, and enhancement of phosphatidylinositol 3-kinase (PtdIns 3-kinase) activity (Cunningham E. et al. (1995) Curr. Biol. 5 (7):775-783; (1995) Nature 377 (6549):544-547; and Panaretou C. et al. (1997) J. Biol. Chem. 272 (4): 2477-2485).

Accordingly, based on its homology with PTDINSTP, rat 19r may modulate potassium channel, e.g., Kv4, activity via the PLC-beta signaling pathway and/or the PtdIns 3-kinase signaling pathway. Rat p19r may also be involved in potassium channel, e.g., Kv4, trafficking.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method for identifying a compound suitable for treating a cardiovascular disorder comprising:
a) contacting a PCIP polypeptide or a fragment thereof, or a cell expressing a PCIP polypeptide or a fragment thereof, with a test compound; and
b) determining whether said PCIP polypeptide or fragment thereof, binds to said test compound, thereby identifying a compound suitable for treating a cardiovascular disorder.

2. The method of claim 1, wherein the binding of said test compound to said PCIP polypeptide or fragment thereof, is detected by a method selected from the group consisting of:
a) detection of binding by direct detection of test compound/polypeptide binding;
b) detection of binding using a competition binding assay; and
c) detection of binding using an assay for PCIP activity.

3. A method for identifying a compound suitable for treating a cardiovascular disorder, comprising:
a) incubating a cell expressing i) a potassium channel comprising a Kv4.3 or Kv4.2 subunit, or a fragment of a potassium channel comprising a Kv4.3 or Kv4.2 subunit, and ii) a PCIP polypeptide or a fragment thereof, in the presence and absence of a candidate compound; and
b) determining whether the presence of the candidate compound modulates the interaction of the potassium channel or fragment thereof with said PCIP polypeptide or fragment thereof, thereby identifying a compound suitable for treating a cardiovascular disorder.

4. A method for treating a cardiovascular disorder comprising contacting a potassium channel with an effective amount of a compound that modulates the binding of a PCIP protein to said potassium channel.

5. A method for determining if a subject is at risk for a cardiovascular disorder comprising detecting, in a sample of cells from the subject an alteration in a PCIP gene which causes a mutated PCIP polypeptide to be produced.

6. A method for determining if a subject is at risk for a cardiovascular disorder comprising detecting, in a sample of cells from the subject an alteration in a PCIP gene which causes abnormal expression of a PCIP polypeptide.

7. A method for determining if a subject is at risk for a cardiovascular disorder comprising detecting, in a sample of cells from the subject an alteration in a PCIP gene which causes abnormal processing of a PCIP polypeptide.

8. A method for identifying a subject suffering from a cardiovascular disorder comprising detecting, in a sample of cells from the subject an alteration in a PCIP gene which causes a mutated PCIP polypeptide to be produced.

9. A method for identifying a subject suffering from a cardiovascular disorder comprising detecting, in a sample of cells from the subject an alteration in a PCIP gene which causes abnormal expression of a PCIP polypeptide.

10. A method for identifying a subject suffering from a cardiovascular disorder comprising detecting, in a sample of cells from the subject an alteration in a PCIP gene which causes abnormal processing of a PCIP polypeptide.

11. The method of any one of claims 1, 3, 4, 5, 6, 7, 8, 9, or 10, wherein said cardiovascular disorder is associated with an abnormal Iₜₒ current.

12. The method of any one of claims 1, 3, 4, 5, 6, 7, 8, 9, or 10, wherein said PCIP is 9q.

13. The method of any one of claims 1, 3, 4, 5, 6, 7, 8, 9, or 10, wherein said PCIP is 8t.

14. The method of any one of claims 1, 3, 4, 5, 6, 7, 8, 9, or 10, wherein said PCIP is p19.

15. The method of any one of claims 1, 3, 4, 5, 6, 7, 8, 9, or 10, wherein said cardiovascular disorder is long-QT syndrome.

16. The method of any one of claims 1, 3, 4, 5, 6, 7, 8, 9, or 10, wherein said cardiovascular disorder is congestive heart failure.


SEQUENCE LISTING (72 sequences)

SEQ ID NO: 1 - DNA, 1463 bp, Homo sapiens
  CDS (225)..(872)
gaatagccccctttcacttctgagtccctgcatgtgcggggctgaagaaggaagccagaagcctcctagcctcgcctccacgtttgctgaataccaagctgcaggcgagctgccgggcgcttttctctcctccaattcagagtagacaaaccacggggatttctttccagggtaggggaggggccgggcccggggtcccaactcgcactcaagtcttcgctgccatgggggccgtcatgggcaccttctcatctctgcaaaccaaacaaaggcgaccctcgaaagataagattgaagatgagctggagatgaccatggtttgccatcggcccgagggactggagcagctcgaggcccagaccaacttcaccaagagggagctgcaggtcctttatcgaggcttcaaaaatgagtgccccagtggtgtggtcaacgaagacacattcaagcagatctatgctcagtttttccctcatggagatgccagcacgtatgcccattacctcttcaatgccttcgacaccactcagacaggctccgtgaagttcgaggactttgtaaccgctctgtcgattttattgagaggaactgtccacgagaaactaaggtggacatttaatttgtatgacatcaacaaggacggatacataaacaaagaggagatgatggacattgtcaaagccatctatgacatgatggggaaatacacatatcctgtgctcaaagaggacactccaaggcagcatgtggacgtcttcttccagaaaatggacaaaaataaagatggcatcgtaactttagatgaatttcttgaatcatgtcaggaggacgacaacatcatgaggtctctccagctgtttcaaaatgtcatgtaactggtgacactcagccattcagctctcagagacattgtactaaacaaccaccttaacaccctgatctgcccttgttctgattttacacaccaactcttgggacagaaacaccttttacactttggaagaattctctgctgaagactttcttatggaacccagcatcatgtggctcagtctctgattgccaactcttcctctttcttcttcttgagagagacaagatgaaatttgagtttgttttggaagcatgctcatctcctcacactgctgccctatggaaggtccctctgcttaagcttaaacagtagtgcacaaaatatgctgcttacgtgcccccagcccactgcctccaagtcaggcagaccttggtgaatctggaagcaagaggacctgagccagatgcacaccatctctgatggcctcccaaaccaatgtgcctgtttctcttcctttggtgggaagaatgagagttatccagaacaattaggatctgtcatgaccagattgggagagccagcacctaacatatgtgggataggactgaattattaagcatgacattgtctgatgacccaaactgccccg

SEQ ID NO: 2 - protein, 216 aa, Homo sapiens
MGAVMGTFSSLQTKQRRPSKDKIEDELEMTMVCHRPEGLEQLEAQTNFTKRELQVLYRGFKNECPSGVVNEDTFKQIYAQFFPHGDASTYAHYLFNAFDTTQTGSVKFEDFVTALSILLRGTVHEKLRWTFNLYDINKDGYINKEEMMDIVKAIYDMMGKYTYPVLKEDTPRQHVDVFFQKMDKNKDGIVTLDEFLESCQEDDNIMRSLQLFQNVM

SEQ ID NO: 3 - DNA, 1856 bp, Rattus sp.
  CDS (300)..(1034)
ggcacacaacccctggattcttcggagaatatgccgtgaggtgttgccaattattagttctcttggctagcagatgtttagggactggttaagcctttggagaaattaccttaggaaaacggggaaataaaagcaaagattaccatgaattgcaagattacctagcaattgcaaggtaggaggagagaggtggagggcggagtagacaggagggagggagaaagtgagaggaagctaggctggtggaaataaccctgcacttggaacagcggcaaagaagcgcgattttccagctttaaatgcctgcccgcgttctgcttgcctacccgggaacggagatgttgacccagggcgagtctgaagggctccagaccttggggatagtagtggtcctgtgttcctctctgaaactactgcactacctcgggctgattgacttgtcggatgacaagatcgaggatgatctggagatgaccatggtttgccatcggcctgagggactggagcagcttgaggcacagacgaacttcaccaagagagaactgcaagtcctttaccggggattcaaaaacgagtgccccagtggtgtggttaacgaagagacattcaagcagatctacgctcagtttttccctcatggagatgccagcacatacgcacattacctcttcaatgccttcgacaccacccagacaggctctgtaaagttcgaggactttgtgactgctctgtcgattttactgagaggaacggtccatgaaaaactgaggtggacgtttaatttgtacgacatcaataaagacggctacataaacaaagaggagatgatggacatagtgaaagccatctatgacatgatggggaaatacacctatcctgtgctcaaagaggacactcccaggcagcacgtggacgtcttcttccagaaaatggataaaaataaagatggcattgtaacgttagacgaatttctcgagtcctgtcaggaggatgacaacatcatgaggtctctacagctgttccaaaatgtcatgtaactgaggacactggccatcctgctctcagagacactgacaaacacctcaatgccctgatctgcccttgttccagttttacacatcaactctcgggacagaaataccttttacactttggaagaattctctgctgaagactttctacaaaacctggcaccgagtggctcagtctctgattgccaactcttcctccctcctcctcttgagagggacgagctgaaatccgaagtttgttttggaagcatgcccatctctccatgctgctgctgccctgtggaaggcccctctgcttgagcttaaacagtagtgcacagttttctgcgtatacagatccccaactcactgcctctaagtcaggcagaccctgatcaatctgaaccaaatgtgcaccatcctccgatggcctcccaagccaatgtgcctgcttctcttcctctggtgggaagaaagaacgctctacagagcacttagagcttaccatgaaaatactgggagaggcagcacctaacacatgtagaataggactgaattattaagcatggtggtatcagatgatgcaaacagcccatgtcatttttttttccagaggtagggactaataattctcccacactagcacctacgatcatagaacaagtcttttaacacatccaggagggaaaccgctgcccagtggtctatcccttctctccatcccctgctcaagcccagcactgcatgtctctcccggaaggtccagaatgcctgtgaaatgctgtaacttttataccctgttataatcaataaacagaactatttcgtacaaaaaaaaaaaaaaaa

SEQ ID NO: 4 - protein, 245 aa, Rattus sp.
MPARVLLAYPGTEMLTQGESEGLQTLGIVVVLCSSLKLLHYLGLIDLSDDKIEDDLEMTMVCHRPEGLEQLEAQTNFTKRELQVLYRGFKNECPSGVVNEETFKQIYAQFFPHGDASTYAHYLFNAFDTTQTGSVKFEDFVTALSILLRGTVHEKLRWTFNLYDINKDGYINKEEMMDIVKAIYDMMGKYTYPVLKEDTPRQHVDVFFQKMDKNKDGIVTLDEFLESCQEDDNIMRSLQLFQNVM

SEQ ID NO: 5 - DNA, 1907 bp, Mus musculus
  CDS (477)..(1124)
cggccccctgagatccagcccgagcgcggggcggagcggccgggtggcagcaggggcgggcgggcggagcgcagctcccgcaccgcacgcggcgcgggctcggcagcctcggccgtgcgggcacgccggccccgtgtccaacatcaggcaggctttggggctcggggctcgggcctcggagaagccagtggcccggctgggtgcccgcaccggggggcgcctgtgaaggctcccgcgagcctctggccctgggagtcagtgcatgtgcctggctgaagaaggcagcagccacgagctccaggcgccccggccccacgttttctgaataccaagctgcaggcgagctgctcggggcttttttgctttctcgcttttcctctcctccaattcaaagtgggcaatccacaccgatttcttttcaggggagggaagagacagggcctggggtcccaagacgcacacaagtcttcgctgccatgggggccgtcatgggcactttctcctccctgcagaccaaacaaaggcgaccctctaaagacaagattgaggatgagctagagatgaccatggtttgccaccggcctgagggactggagcagcttgaggcacagacgaacttcaccaagagagaactgcaagtcttgtaccggggattcaaaaacgagtgccctagcggtgtggtcaatgaagaaacattcaagcagatctacgctcagtttttccctcacggagatgccagcacatatgcacattacctcttcaatgccttcgacaccacccagacaggctctgtaaagttcgaggactttgtgactgctctgtcgattttactgagagggacagtccatgaaaaactaaggtggacgtttaatttgtatgacatcaataaagacggctacataaacaaagaggagatgatggacatagtcaaagccatctatgacatgatggggaaatacacctatcctgtgctcaaagaggacactcccaggcagcatgtggatgtcttcttccagaaaatggataaaaataaagatggcattgtaacgttagatgaatttcttgaatcatgtcaggaggatgacaacatcatgagatctctacagctgttccaaaatgtcatgtaactgaggacactggccattctgctctcagagacactgacaaacaccttaatgccctgatctgcccttgttccaattttacacaccaactcttgggacagaaataccttttacactttggaagaattctctgctgaagactttctacaaaacctggcaccacgtggctctgtctctgagggacgagcggagatccgactttgttttggaagcatgcccatctcttcatgctgctgccctgtggaaggcccctctgcttgagcttaatcaatagtgcacagttttatgcttacacatatccccaactcactgcctccaagtcaggcagactctgatgaatctgagccaaatgtgcaccatcctccgatggcctcccaagccaatgtgcctgcttctcttcctctggtgggaagaaagagtgttctacggaacaattagagcttaccatgaaaatattgggagaggcagcacctaacacatgtagaataggactgaattattaagcatggtgatatcagatgatgcaaattgcccatgtcatttttttcaaaggtagggacaaatgattctcccacactagcacctgtggtcatagagcaagtctcttaacatgcccagaaggggaaccactgtccagtggtctatccctcctctccatcccctgctcaaacccagcactgcatgtccctccaagaaggtccagaatgcctgcgaaacgctgtacttttataccctgttctaatcaataaacagaactatttcgtaaaaaaaaaaaaaaaaaaa

SEQ ID NO: 6 - protein, 216 aa, Mus musculus
MGAVMGTFSSLQTKQRRPSKDKIEDELEMTMVCHRPEGLEQLEAQTNFTKRELQVLYRGFKNECPSGVVNEETFKQIYAQFFPHGDASTYAHYLFNAFDTTQTGSVKFEDFVTALSILLRGTVHEKLRWTFNLYDINKDGYINKEEMMDIVKAIYDMMGKYTYPVLKEDTPRQHVDVFFQKMDKNKDGIVTLDEFLESCQEDDNIMRSLQLFQNVM

SEQ ID NO: 7 - DNA, 1534 bp, Rattus sp.
  CDS (31)..(711)
gtcccaagtcgcacacaagtcttcgctgccatgggggccgtcatgggtaccttctcgtccctgcagaccaaacaaaggcgaccctctaaagacatcgcctggtggtattaccagtatcagagagacaagatcgaggatgatctggagatgaccatggtttgccatcggcctgagggactggagcagcttgaggcacagacgaacttcaccaagagagaactgcaagtcctttaccggggattcaaaaacgagtgccccagtggtgtggttaacgaagagacattcaagcagatctacgctcagtttttccctcatggagatgccagcacatacgcacattacctcttcaatgccttcgacaccacccagacaggctctgtaaagttcgaggactttgtgactgctctgtcgattttactgagaggaacggtccatgaaaaactgaggtggacgtttaatttgtacgacatcaataaagacggctacataaacaaagaggagatgatggacatagtgaaagccatctatgacatgatggggaaatacacctatcctgtgctcaaagaggacactcccaggcagcacgtggacgtcttcttccagaaaatggataaaaataaagatggcattgtaacgttagacgaatttctcgagtcctgtcaggaggatgacaacatcatgaggtctctacagctgttccaaaatgtcatgtaactgaggacactggccatcctgctctcagagacactgacaaacacctcaatgccctgatctgcccttgttccagttttacacatcaactctcgggacagaaataccttttacactttggaagaattctctgctgaagactttctacaaaacctggcaccgcgtggctcagtctctgattgccaactcttcctccctcctcctcttgagagggacgagctgaaatccgaagtttgttttggaagcatgcccatctctccatgctgctgctgccctgtggaaggcccctctgcttgagcttaaacagtagtgcacagttttctgcgtatacagatccccaactcactgcctctaagtcaggcagaccctgatcaatctgaaccaaatgtgcaccatcctccgatggcctcccaagccaatgtgcctgcttctcttcctctggtgggaagaaagaacgctctacagagcacttagagcttaccatgaaaatactgggagaggcagcacctaacacatgtagaataggactgaattattaagcatggtggtatcagatgatgcaaacagcccatgtcattttttttccagaggtagggactaataattctcccacactagcacctacgatcatagaacaagtcttttaacacatccaggagggaaaccgctgcccagtggtctatcccttctctccatcccctgctcaagcccagcactgcatgtctctcccggaaggtccagaatgcctgtgaaatgctgtaacttttataccctgttataatcaataaacagaactatttcgtacaaaaaaaaaaaaaaaaaa

SEQ ID NO: 8 - protein, 227 aa, Rattus sp.
MGAVMGTFSSLQTKQRRPSKDIAWWYYQYQRDKIEDDLEMTMVCHRPEGLEQLEAQTNFTKRELQVLYRGFKNECPSGVVNEETFKQIYAQFFPHGDASTYAHYLFNAFDTTQTGSVKFEDFVTALSILLRGTVHEKLRWTFNLYDINKDGYINKEEMMDIVKAIYDMMGKYTYPVLKEDTPRQHVDVFFQKMDKNKDGIVTLDEFLESCQEDDNIMRSLQLFQNVM

SEQ ID NO: 9 - DNA, 1540 bp, Mus musculus
  CDS (77)..(757)
atccacaccgatttcttttcaggggagggaagagacagggcctggggtcccaagacgcacacaagtcttcgctgccatgggggccgtcatgggcactttctcctccctgcagaccaaacaaaggcgaccctctaaagacatcgcctggtggtattaccagtatcagagagacaagattgaggatgagctagagatgaccatggtttgccaccggcctgagggactggagcagcttgaggcacagacgaacttcaccaagagagaactgcaagtcttgtaccggggattcaaaaacgagtgccctagcggtgtggtcaatgaagaaacattcaagcagatctacgctcagtttttccctcacggagatgccagcacatatgcacattacctcttcaatgccttcgacaccacccagacaggctctgtaaagttcgaggactttgtgactgctctgtcgattttactgagagggacagtccatgaaaaactaaggtggacgtttaatttgtatgacatcaataaagacggctacataaacaaagaggagatgatggacatagtcaaagccatctatgacatgatggggaaatacacctatcctgtgctcaaagaggacactcccaggcagcatgtggatgtcttcttccagaaaatggataaaaataaagatggcattgtaacgttagatgaatttcttgaatcatgtcaggaggatgacaacatcatgagatctctacagctgttccaaaatgtcatgtaactgaggacactggccattctgctctcagagacactgacaaacaccttaatgccctgatctgcccttgttccaattttacacaccaactcttgggacagaaataccttttacactttggaagaattctctgctgaagactttctacaaaacctggcaccacgtggctctgtctctgagggacgagcggagatccgactttgttttggaagcatgcccatctcttcatgctgctgccctgtggaaggcccctctgcttgagcttaatcaatagtgcacagttttatgcttacacatatccccaactcactgcctccaagtcaggcagactctgatgaatctgagccaaatgtgcaccatcctccgatggcctcccaagccaatgtgcctgcttctcttcctctggtgggaagaaagagtgttctacggaacaattagagcttaccatgaaaatattgggagaggcagcacctaacacatgtagaataggactgaattattaagcatggtgatatcagatgatgcaaattgcccatgtcatttttttcaaaggtagggacaaatgattctcccacactagcacctgtggtcatagagcaagtctcttaacatgcccagaaggggaaccactgtccagtggtctatccctcctctccatcccctgctcaaacccagcactgcatgtccctccaagaaggtccagaatgcctgcgaaacgctgtacttttataccctgttctaatcaataaacagaactatttcgtacaaaaaaaaaaaaaaaaa

SEQ ID NO: 10 - protein, 227 aa, Mus musculus
MGAVMGTFSSLQTKQRRPSKDIAWWYYQYQRDKIEDELEMTMVCHRPEGLEQLEAQTNFTKRELQVLYRGFKNECPSGVVNEETFKQIYAQFFPHGDASTYAHYLFNAFDTTQTGSVKFEDFVTALSILLRGTVHEKLRWTFNLYDINKDGYINKEEMMDIVKAIYDMMGKYTYPVLKEDTPRQHVDVFFQKMDKNKDGIVTLDEFLESCQEDDNIMRSLQLFQNVM

SEQ ID NO: 11 - DNA, 955 bp, Rattus sp.
  CDS (345)..(953)
  Xaa at position 92 of the corresponding amino acid sequence may be any amino acid
gtccgggcacacaacccctggattcttcggagaatatgccgtgacggtgttgccaattattagttctcttggctagcagatgtttagggactggttaagcctttggagaaattaccttaggaaaacggggaaataaaagcaaagattaccatgaattgcaagattacctagcaattgcaaggtaggaggagagaggtggagggcggagtagacaggagggagggagaaagtgagaggaagctaggctggtggaaataaccctgcacttggaacagcggcaaagaagcgcgattttccagctttaaatgcctgcccgcgttctgcttgcctacccgggaacggagatgttgacccagggcgagtctgaagggctccagaccttggggatagtagtggtcctgtgttcctctctgaaactactgcactacctcgggctgattgacttgtcggatgacaagatcgaggatgatctggagatgaccatggtttgccatcggcctgagggactggagcagcttgaggcacagacgaacttcaccaagagagaactgcaagtcctttaccggggattcaaaaacgagtgccccagtggtgtggttaacgaagagacattcaagcngatctacgctcagtttttccctcatggagatgccagcacatacgcacattacctcttcaatgccttcgacaccacccagacaggctctgtaaagttcgaggactttgtgactgctctgtcgattttactgagaggaacggtccatgaaaaactgaagtggacgtttaatttgtacgacatcaataaagacggctacataaacaaagaggagatgatggacatagtgaaagccatctatgacatgatggggaaatacacctatcttgtgctcaaagaggacacttccaggcagcacgtggacgtcttcttccagaaaatggataaaaataaagatgg

SEQ ID NO: 12 - protein, 203 aa, Rattus sp.
MLTQGESEGLQTLGIVVVLCSSLKLLHYLGLIDLSDDKIEDDLEMTMVCHRPEGLEQLEAQTNFTKRELQVLYRGFKNECPSGVVNEETFKXIYAQFFPHGDASTYAHYLFNAFDTTQTGSVKFEDFVTALSILLRGTVHEKLKWTFNLYDINKDGYINKEEMMDIVKAIYDMMGKYTYLVLKEDTSRQHVDVFFQKMDKNKD

SEQ ID NO: 13 - DNA, 2009 bp, Homo sapiens
  CDS (207)..(1016)
ctcacctgctgcctagtgttccctctcctgctccaggacctccgggtagacctcagaccccgggcccattcccagactcagcctcagcccggacttccccagccccgacagcacagtaggccgccagggggcgccgtgtgagcgccctatcccggccacccggcgccccctcccacggcccgggcgggagcggggcgccgggggccatgcggggccagggccgcaaggagagtttgtccgattcccgagacctggacggctcctacgaccagctcacgggccaccctccagggcccactaaaaaagcgctgaagcagcgattcctcaagctgctgccgtgctgcgggccccaagccctgccctcagtcagtgaaacattagccgccccagcctccctccgcccccacagaccccgcctgctggacccagacagcgtggacgatgaatttgaattgtccaccgtgtgtcaccggcctgagggtctggagcagctgcaggagcaaaccaaattcacgcgcaaggagttgcaggtcctgtaccggggcttcaagaacgaatgtcccagcggaattgtcaatgaggagaacttcaagcagatttactcccagttctttcctcaaggagactccagcacctatgccacttttctcttcaatgcctttgacaccaaccatgatggctcggtcagttttgaggactttgtggctggtttgtccgtgattcttcggggaactgtagatgacaggcttaattgggccttcaacctgtatgaccttaacaaggacggctgcatcaccaaggaggaaatgcttgacatcatgaagtccatctatgacatgatgggcaagtacacgtaccctgcactccgggaggaggccccaagggaacacgtggagagcttcttccagaagatggacagaaacaaggatggtgtggtgaccattgaggaattcattgagtcttgtcaaaaggatgagaacatcatgaggtccatgcagctctttgacaatgtcatctagcccccaggagagggggtcagtgtttcctggggggaccatgctctaaccctagtccaggcggacctcacccttctcttcccaggtctatcctcatcctacgcctccctgggggctggagggatccaagagcttggggattcagtagtccagatctctggagctgaaggggccagagagtgggcagagtgcatctcggggggtgttcccaactcccaccagctctcacccccttcctgcctgacacccagtgttgagagtgcccctcctgtaggaattgagcggttccccacctcctaccctactctagaaacacactagagcgatgtctcctgctatggtgcttcccccatccctgacctcataaacatttcccctaagactcccctctcagagagaatgctccattcttggcactggctggcttctcagaccagccattgagagccctgtgggagggggacaagaatgtatagggagaaatcttgggcctgagtcaatggataggtcctaggaggtgggtggggttgagaatagaagggcctggacagattatgattgctcaggcataccaggttatagctccaagttccacaggtctgctaccacaggccatcaaaatataagtttccaggctttgcagaagaccttgtctccttagaaatgccccagaaattttccacaccctcctcggtatccatggagagcctggggccagatatctggctcatctctggcattgcttcctctccttccttcctgcatgtgttggtggtggttgtggtgggggaatgtggatgggggatgtcctggctgatgcctgccaaaatttcatcccaccctccttgcttatcgtccctgttttgagggctatgacttgagtttttgtttcccatgttctctatagacttgggaccttcctgaacttggggcctatcactccccacagtggatgccttagaagggagagggaaggagggaggcaggcatagc

SEQ ID NO: 14 - protein, 270 aa, Homo sapiens
MRGQGRKESLSDSRDLDGSYDQLTGHPPGPTKKALKQRFLKLLPCCGPQALPSVSETLAAPASLRPHRPRLLDPDSVDDEFELSTVCHRPEGLEQLQEQTKFTRKELQVLYRGFKNECPSGIVNEENFKQIYSQFFPQGDSSTYATFLFNAFDTNHDGSVSFEDFVAGLSVILRGTVDDRLNWAFNLYDLNKDGCITKEEMLDIMKSIYDMMGKYTYPALREEAPREHVESFFQKMDRNKDGVVTIEEFIESCQKDENIMRSMQLFDNVI

SEQ ID NO: 15 - DNA, 1247 bp, Rattus sp.
  CDS (2)..(772)
ccgagatctggacggctcctatgaccagcttacgggccaccctccagggcccagtaaaaaagccctgaagcagcgtttcctcaagctgctgccgtgctgcgggccccaagccctgccctcagtcagtgaaacattagctgccccagcctccctccgcccccacagaccccgcccgctggacccagacagcgtagaggatgagtttgaattatccacggtgtgtcaccgacctgagggcctggaacaactccaggaacagaccaagttcacacgcagagagctgcaggtcctgtaccgaggcttcaagaacgaatgccccagtgggattgtcaacgaggagaacttcaagcagatttattctcagttctttccccaaggagactccagcaactatgctacttttctcttcaatgcctttgacaccaaccacgatggctctgtcagttttgaggactttgtggctggtttgtcggtgattcttcgggggaccatagatgatagactgagctgggctttcaacttatatgacctcaacaaggacggctgtatcacaaaggaggaaatgcttgacattatgaagtccatctatgacatgatgggcaagtacacataccctgccctccgggaggaggccccaagagaacacgtggagagcttcttccagaagatggacaggaacaaggacggcgtggtgaccatcgaggaattcatcgagtcttgtcaacaggacgagaacatcatgaggtccatgcagctctttgataatgtcatctagctccccagggagaggggttagtgtgtcctagggtgaccaggctgtagtcctagtccagacgaacctaaccctctctctccaggcctgtcctcatcttacctgtaccctgggggctgtagggattcaatatcctggggcttcagtagtccagatccctgagctaagtcacaaaagtaggcaagagtaggcaagctaaatctgggggcttcccaacccccgacagctctcaccccttctcaactgatacctagtgctgaggacacccctggtgtagggaccaagtggttctccaccttctagtcccactctagaaaccacattagacagaaggtctcctgctatggtgctttccccatccctaatctcttagattttcctcaagactcccttctcagagaacacgctctgtccatgtccccagctggggacatggacagagcgtgttctctagttctagatcgcgagcggccgc

SEQ ID NO: 16 - protein, 257 aa, Rattus sp.
RDLDGSYDQLTGHPPGPSKKALKQRFLKLLPCCGPQALPSVSETLAAPASLRPHRPRPLDPDSVEDEFELSTVCHRPEGLEQLQEQTKFTRRELQVLYRGFKNECPSGIVNEENFKQIYSQFFPQGDSSNYATFLFNAFDTNHDGSVSFEDFVAGLSVILRGTIDDRLSWAFNLYDLNKDGCITKEEMLDIMKSIYDMMGKYTYPALREEAPREHVESFFQKMDRNKDGVVTIEEFIESCQQDENIMRSMQLFDNVI

SEQ ID NO: 17 - DNA, 2343 bp, Mus musculus
  CDS (181)..(990)
cgggactctgaggtgggccctaaaatccagcgctccccagagaaaagccttgccagcccctactcccggcccccagccccagcaggtcgctgcgccgccagggggcactgtgtgagcgccctatcctggccacccggcgccccctcccacggcccaggcgggagcggggcgccgggggccatgcggggccaaggccgaaaggagagtttgtccgaatcccgagatttggacggctcctatgaccagcttacgggccaccctccagggcccagtaaaaaagccctgaagcagcgtttcctcaagctgctgccgtgctgcgggccccaagccctgccctcagtcagtgaaacattagctgccccagcctccctccgcccccacagaccccgcccgctggacccagacagcgtggaggatgagtttgaactatccacggtgtgccaccggcctgagggtctggaacaactccaggaacaaaccaagttcacacgcagagagttgcaggtcctgtacagaggcttcaagaacgaatgtcccagcggaattgtcaacgaggagaacttcaagcaaatttattctcagttctttccccaaggagactccagcaactacgctacttttctcttcaatgcctttgacaccaaccatgatggctctgtcagttttgaggactttgtggctggtttgtcagtgattcttcggggaaccatagatgatagactgaactgggctttcaacttatatgacctcaacaaggatggctgtatcacgaaggaggaaatgctcgacatcatgaagtccatctatgacatgatgggcaagtacacctaccctgccctccgggaggaggccccgagggaacacgtggagagcttcttccagaagatggacagaaacaaggacggcgtggtgaccattgaggaattcattgagtcttgtcaacaggacgagaacatcatgaggtccatgcaactctttgataatgtcatctagctccccagggagaggggttagtgtgtcccagggtaaccatgctgtagccctagtccaggcaaacctaaccctcctctccccgggtctgtcctcatcctacctgtaccctgggggctgtagggattcaacatcctggcgcttcagtagtccagatccctgagctaagtggcgagagtaggcaagctaagtctttggagggtgggtgggggcgcgcagattcccaacccccgacgactctcacccctttctcgactgatacccagtgctgaggctacccctggtgtcgggaacgaccaaagtggttctctgcctccccagcccactctagagacccacactagacgggaatatctcctgctatggtgctttccccatccctgaccgcagattttcctcctaagactcccttctcagagaatatgcttttgtcccttgtccctggctggcttttcagcctagcctttgaggaccctgtgggaggggagaataagaaagcagacaaaatcttggccctgagccagtggttaggtcctaggaatcaggctggagtggagaccagaaagcctgggcaggctatgagagccccaggttggcttgtcaccgccaggttccacagggctgctgctctgggtcagcagagtatgagtttccagactttccagaaggccttatgtccttagcaatgtcccagaaattcaccatacacttctcagtgtcttaggatccagatgtccggtccatccctgaaacctctccctcctccttgctcctatggtgggagtggtggccaggggacgatgagtgagccggtgtcctggatgatgcctgtcaaggtcccacctaccctccggctgtcaagccgttctggtgaccctgtttgattctccatgacccctgtctagatgtagaggtgtggagtgagtctagtggcagccttaggggaatgggaagaacgagaggggcactccatctgaacccagtgtgggggcatccattcgaatctttgcctggctccccacaatgccctaggatcctctagggtccccacccccactctttagtctacccagagatgctccagagctcacctagagggcagggaccataggatccaggtccaacctgtcatcagcatccggccatgctgctgctgcttattaataaacctgcttgtcgttcagcgccccttcccagtcagccagggtctgaggggaaggcccccactttcccgcctcctgtcagacattgttgactgctttgcattttgggctcttctacctatattttgtataataagaaagacaccagatccaataaaacacatggctatgcacaaaaaaaaaaaaaaaa

SEQ ID NO: 18 - protein, 270 aa, Mus musculus
MRGQGRKESLSESRDLDGSYDQLTGHPPGPSKKALKQRFLKLLPCCGPQALPSVSETLAAPASLRPHRPRPLDPDSVEDEFELSTVCHRPEGLEQLQEQTKFTRRELQVLYRGFKNECPSGIVNEENFKQIYSQFFPQGDSSNYATFLFNAFDTNHDGSVSFEDFVAGLSVILRGTIDDRLNWAFNLYDLNKDGCITKEEMLDIMKSIYDMMGKYTYPALREEAPREHVESFFQKMDRNKDGVVTIEEFIESCQQDENIMRSMQLFDNVI

SEQ ID NO: 19 - DNA, 1955 bp, Homo sapiens
  CDS (207)..(962)
ctcacctgctgcctagtgttccctctcctgctccaggacctccgggtagacctcagaccccgggcccattcccagactcagcctcagcccggacttccccagccccgacagcacagtaggccgccagggggcgccgtgtgagcgccctatcccggccacccggcgccccctcccacggcccgggcgggagcggggcgccgggggccatgcggggccagggccgcaaggagagtttgtccgattcccgagacctggacggctcctacgaccagctcacgggccaccctccagggcccactaaaaaagcgctgaagcagcgattcctcaagctgctgccgtgctgcgggccccaagccctgccctcagtcagtgaaaacagcgtggacgatgaatttgaattgtccaccgtgtgtcaccggcctgagggtctggagcagctgcaggagcaaaccaaattcacgcgcaaggagttgcaggtcctgtaccggggcttcaagaacgaatgtcccagcggaattgtcaatgaggagaacttcaagcagatttactcccagttctttcctcaaggagactccagcacctatgccacttttctcttcaatgcctttgacaccaaccatgatggctcggtcagttttgaggactttgtggctggtttgtccgtgattcttcggggaactgtagatgacaggcttaattgggccttcaacctgtatgaccttaacaaggacggctgcatcaccaaggaggaaatgcttgacatcatgaagtccatctatgacatgatgggcaagtacacgtaccctgcactccgggaggaggccccaagggaacacgtggagagcttcttccagaagatggacagaaacaaggatggtgtggtgaccattgaggaattcattgagtcttgtcaaaaggatgagaacatcatgaggtccatgcagctctttgacaatgtcatctagcccccaggagagggggtcagtgtttcctggggggaccatgctctaaccctagtccaggcggacctcacccttctcttcccaggtctatcctcatcctacgcctccctgggggctggagggatccaagagcttggggattcagtagtccagatctctggagctgaaggggccagagagtgggcagagtgcatctcggggggtgttcccaactcccaccagctctcacccccttcctgcctgacacccagtgttgagagtgcccctcctgtaggaattgagcggttccccacctcctaccctactctagaaacacactagagcgatgtctcctgctatggtgcttcccccatccctgacctcataaacatttcccctaagactcccctctcagagagaatgctccattcttggcactggctggcttctcagaccagccattgagagccctgtgggagggggacaagaatgtatagggagaaatcttgggcctgagtcaatggataggtcctaggaggtgggtggggttgagaatagaagggcctggacagattatgattgctcaggcataccaggttatagctccaagttccacaggtctgctaccacaggccatcaaaatataagtttccaggctttgcagaagaccttgtctccttagaaatgccccagaaattttccacaccctcctcggtatccatggagagcctggggccagatatctggctcatctctggcattgcttcctctccttccttcctgcatgtgttggtggtggttgtggtgggggaatgtggatgggggatgtcctggctgatgcctgccaaaatttcatcccaccctccttgcttatcgtccctgttttgagggctatgacttgagtttttgtttcccatgttctctatagacttgggaccttcctgaacttggggcctatcactccccacagtggatgccttagaagggagagggaaggagggaggcaggcatagc

SEQ ID NO: 20 - protein, 252 aa, Homo sapiens
MRGQGRKESLSDSRDLDGSYDQLTGHPPGPTKKALKQRFLKLLPCCGPQALPSVSENSVDDEFELSTVCHRPEGLEQLQEQTKFTRKELQVLYRGFKNECPSGIVNEENFKQIYSQFFPQGDSSTYATFLFNAFDTNHDGSVSFEDFVAGLSVILRGTVDDRLNWAFNLYDLNKDGCITKEEMLDIMKSIYDMMGKYTYPALREEAPREHVESFFQKMDRNKDGVVTIEEFIESCQKDENIMRSMQLFDNVI

SEQ ID NO: 21 - DNA, 2300 bp, Rattus sp.
  CDS (214)..(969)
ctcacttgctgcccaaggctcctgctcctgccccaggactctgaggtgggccctaaaacccagcgctctctaaagaaaagccttgccagcccctactcccggcccccaaccccagcaggtcgctgcgccgccagggggcgctgtgtgagcgccctattctggccacccggcgccccctcccacggcccaggcgggagcggggcgccgggggccatgcggggccaaggcagaaaggagagtttgtccgaatcccgagatctggacggctcctatgaccagcttacgggccaccctccagggcccagtaaaaaagccctgaagcagcgtttcctcaagctgctgccgtgctgcgggccccaagccctgccctcagtcagtgaaaacagcgtagaggatgagtttgaattatccacggtgtgtcaccgacctgagggcctggaacaactccaggaacagaccaagttcacacgcagagagctgcaggtcctgtaccgaggcttcaagaacgaatgccccagtgggattgtcaacgaggagaacttcaagcagatttattctcagttctttccccaaggagactccagcaactatgctacttttctcttcaatgcctttgacaccaaccacgatggctctgtcagttttgaggactttgtggctggtttgtcggtgattcttcgggggaccatagatgatagactgagctgggctttcaacttatatgacctcaacaaggacggctgtatcacaaaggaggaaatgcttgacattatgaagtccatctatgacatgatgggcaagtacacataccctgccctccgggaggaggccccaagagaacacgtggagagcttcttccagaagatggacaggaacaaggacggcgtggtgaccatcgaggaattcatcgagtcttgtcaacaggacgagaacatcatgaggtccatgcagctctttgataatgtcatctagctccccagggagaggggttagtgtgtcctagggtgaccaggctgtagtcctagtccagacgaacctaaccctctctctccaggcctgtcctcatcttacctgtaccctgggggctgtagggattcaatatcctggggcttcagtagtccagatccctgagctaagtcacaaaagtaggcaagagtaggcaagctaaatctgggggcttcccaacccccgacagctctcaccccttctcaactgatacctagtgctgaggacacccctggtgtagggaccaagtggttctccaccttctagtcccactctagaaaccacattagacagaaggtctcctgctatggtgctttccccatccctaatctcttagattttcctcaagactcccttctcagagaacacgctctgtccatgtccccagctggcttctcagcctagcctttgagggccctgtggggaggcggggacaagaaagcagaaaagtcttggccccgagccagtggttaggtcctaggaattggctggagtggaggccagaaagcctgggcagatgatgagagcccagctgggctgtcactgcaggttccggggcctacagccctgggtcagcagagtatgagttcccagactttccagaaggtccttagcaatgtcccagaaattcaccgtacacttctcagtgtcttaggagggcccgggatccagatgtctggttcatccctgaatcctctccctccttcttgctcgtatggtgggagtggtggccaggggaagatgagtggtgtcccggatgatgcctgtcaaggtcccacctcccctccggctgttctcatgacagctgtttggttctccatgacccctatctagatgtagaggcatggagtgagtcagggatttcccgaacttgagttttaccactcctcctagtggctgccttaggggaatgggaagaacccagtgtgggggcacccattagaatctttgcccggctcctcacaatgccctagggtcccctagggtacccgctccctctgtttagtctacccagagatgctcctgagctcacctagagggtagggacggtaggctccaggtccaacctctccaggtcagcaccctgccatgctgctgctcctcattaacaaacctgcttgtctcctcctgcgccccttctcagtcagccagggtctgaggggaagggcctcccgtttccccatccgtcagacatggttgactgctttgcattttgggctcttctatctattttgtaaaataagacatcagatccaataaaacacacggctatgcacaaaaaaaaaaaaaaaaaa

SEQ ID NO: 22 - protein, 252 aa, Rattus sp.
MRGQGRKESLSESRDLDGSYDQLTGHPPGPSKKALKQRFLKLLPCCGPQALPSVSENSVEDEFELSTVCHRPEGLEQLQEQTKFTRRELQVLYRGFKNECPSGIVNEENFKQIYSQFFPQGDSSNYATFLFNAFDTNHDGSVSFEDFVAGLSVILRGTIDDRLSWAFNLYDLNKDGCITKEEMLDIMKSIYDMMGKYTYPALREEAPREHVESFFQKMDRNKDGVVTIEEFIESCQQDENIMRSMQLFDNVI

SEQ ID NO: 23 - DNA, 1859 bp, Homo sapiens
  CDS (207)..(866)
ctcacctgctgcctagtgttccctctcctgctccaggacctccgggtagacctcagaccccgggcccattcccagactcagcctcagcccggacttccccagccccgacagcacagtaggccgccagggggcgccgtgtgagcgccctatcccggccacccggcgccccctcccacggcccgggcgggagcggggcgccgggggccatgcggggccagggccgcaaggagagtttgtccgattcccgagacctggacggctcctacgaccagctcacggacagcgtggacgatgaatttgaattgtccaccgtgtgtcaccggcctgagggtctggagcagctgcaggagcaaaccaaattcacgcgcaaggagttgcaggtcctgtaccggggcttcaagaacgaatgtcccagcggaattgtcaatgaggagaacttcaagcagatttactcccagttctttcctcaaggagactccagcacctatgccacttttctcttcaatgcctttgacaccaaccatgatggctcggtcagttttgaggactttgtggctggtttgtccgtgattcttcggggaactgtagatgacaggcttaattgggccttcaacctgtatgaccttaacaaggacggctgcatcaccaaggaggaaatgcttgacatcatgaagtccatctatgacatgatgggcaagtacacgtaccctgcactccgggaggaggccccaagggaacacgtggagagcttcttccagaagatggacagaaacaaggatggtgtggtgaccattgaggaattcattgagtcttgtcaaaaggatgagaacatcatgaggtccatgcagctctttgacaatgtcatctagcccccaggagagggggtcagtgtttcctggggggaccatgctctaaccctagtccaggcggacctcacccttctcttcccaggtctatcctcatcctacgcctccctgggggctggagggatccaagagcttggggattcagtagtccagatctctggagctgaaggggccagagagtgggcagagtgcatctcggggggtgttcccaactcccaccagctctcacccccttcctgcctgacacccagtgttgagagtgcccctcctgtaggaattgagcggttccccacctcctaccctactctagaaacacactagagcgatgtctcctgctatggtgcttcccccatccctgacctcataaacatttcccctaagactcccctctcagagagaatgctccattcttggcactggctggcttctcagaccagccattgagagccctgtgggagggggacaagaatgtatagggagaaatcttgggcctgagtcaatggataggtcctaggaggtgggtggggttgagaatagaagggcctggacagattatgattgctcaggcataccaggttatagctccaagttccacaggtctgctaccacaggccatcaaaatataagtttccaggctttgcagaagaccttgtctccttagaaatgccccagaaattttccacaccctcctcggtatccatggagagcctggggccagatatctggctcatctctggcattgcttcctctccttccttcctgcatgtgttggtggtggttgtggtgggggaatgtggatgggggatgtcctggctgatgcctgccaaaatttcatcccaccctccttgcttatcgtccctgttttgagggctatgacttgagtttttgtttcccatgttctctatagacttgggaccttcctgaacttggggcctatcactccccacagtggatgccttagaagggagagggaaggagggaggcaggcatagc

SEQ ID NO: 24 - protein, 220 aa, Homo sapiens
MRGQGRKESLSDSRDLDGSYDQLTDSVDDEFELSTVCHRPEGLEQLQEQTKFTRKELQVLYRGFKNECPSGIVNEENFKQIYSQFFPQGDSSTYATFLFNAFDTNHDGSVSFEDFVAGLSVILRGTVDDRLNWAFNLYDLNKDGCITKEEMLDIMKSIYDMMGKYTYPALREEAPREHVESFFQKMDRNKDGVVTIEEFIESCQKDENIMRSMQLFDNVI

SEQ ID NO: 25 - DNA, 2191 bp, Simian sp.
  CDS (133)..(792)
cccacgcgtccgcccacgcgtccgcggacgcgtggggtgcactaggccgccagggggcgccgtgtgagcgccctatcccggccacccggcgccccctcccacggaccgggcgggagcggggcgccgggggccatgcggggccagggccgcaaggagagtttgtccgattcccgagacctggacggatcctacgaccagctcacggacagcgtggaggatgaatttgaattgtccaccgtgtgtcaccggcctgagggtctggagcagctgcaggagcaaaccaaattcacgcgcaaggagttgcaggtcctgtaccggggcttcaagaacgaatgtccgagcggaattgtcaatgaggagaacttcaagcaaatttactcccagttctttcctcaaggagactccagcacctatgccacttttctcttcaatgcctttgacaccaaccatgatggctcggtcagttttgaggactttgtggctggtttgtccgtgattcttcggggaactgtagatgacaggcttaattgggccttcaacttgtatgacctcaacaaggacggctgcatcaccaaggaggaaatgcttgacatcatgaagtccatctatgacatgatgggcaagtacacataccctgcactccgggaggaggccccaagggaacatgtggagaacttcttccagaagatggacagaaacaaggatggcgtggtgaccattgaggaattcattgagtcttgtcaaaaggatgagaacatcatgaggtccatgcagctctttgacaatgtcatctagcccccaggagagggggtcagtgtttcctggggggaccatgctctaaccctagtccaggtggacctcacccttctcttcccaggtctatccttgtcctaggcctccctgggggctggagggatccaagagcttggggattcagtagtccagatctctggagctgaaggggccagagagtgggcagagtgcatcttggggggtgttcccaactcccaccagctttcacccgcttcctgcctgacacccagtgttgagagtgcccctcctgtaggaactgagtggttccccacctcctacccccactctagaaacacactagacagatgtctcctgctatggtgcttcccccatccctgacttcataaacatttcccctaaaactcccttctcagagagaatgctccattcttggcactggctggcttctcagaccagcctttgagagccctgtgggagggggacaagaatgtataggggagaaatcttgggcctgagtcaatggataggtcctaggaggtggctggggttgagaatagaaaggcctggacacaatgtgattgctcaggcataccaagttatagctccaagttccacaggtctgctaccacaggccatcaaaatataagtttccaggctttgcagaagaccttgtctccttggaaatgccccagatattttccataccctcctcgatatccatggagagcctggggctagatatctggcatatccctggcattgcttcctctccttccttcctgcatgtgttggtggtggttgtggcaggggaatgtggataggagatgtcctggcagatgcctgccaaagtttcatcccaccctccctgctcatcgcccctgttttgagggctgtgacttgagtttttgtttcccatgttctctatagacttgggaccttcctgaacttggggcctatcactccccacagtggatgccttagaagggagagggaaggagggaggcaggcatagcatctgaacccagtgtgggggcattcactaggatcttcaatcaacccgggctctccccaaccccccagataacctcctcagttccctagagtctcctcttgctctactcaatctacccagagatgccccttagcacactcagagggcagggaccataggacccaggttccaaccccattgtcagcaccccagccatgctgccatcccttagcacacctgctcgtcccattcagcttaccctcccagtcagccagaatctgaggggagggcccccagagagcccccttccccatcagaagactgttgactgctttgcattttgggctcttctatatattttgtaaaataagaactataccagatctaataaaacacaatggctatgcaaaaaaaaaaaaaaaaaaa

SEQ ID NO: 26 - protein, 220 aa, Simian sp.
MRGQGRKESLSDSRDLDGSYDQLTDSVEDEFELSTVCHRPEGLEQLQEQTKFTRKELQVLYRGFKNECPSGIVNEENFKQIYSQFFPQGDSSTYATFLFNAFDTNHDGSVSFEDFVAGLSVILRGTVDDRLNWAFNLYDLNKDGCITKEEMLDIMKSIYDMMGKYTYPALREEAPREHVENFFQKMDRNKDGVVTIEEFIESCQKDENIMRSMQLFDNVI

SEQ ID NO: 27 - DNA, 2057 bp, Simian sp.
  CDS (208)..(963)
tgctgcccaaggctcctgctcctgccccaggactctgaggtgggccctaaaacccagcgctctctaaagaaaagccttgccagcccctactcccggcccccaaccccagcaggtcgctgcgccgccagggggcgctgtgtgagcgccctattctggccacccggcgccccctcccacggcccaggcgggagcggggcgccgggggccatgcggggccaaggcagaaaggagagtttgtccgaatcccgagatctggacggctcctatgaccagcttacgggccaccctccagggcccagtaaaaaagccctgaagcagcgtttcctcaagctgctgccgtgctgcgggccccaagccctgccctcagtcagtgaaaacagcgtagaggatgagtttgaattatccacggtgtgtcaccgacctgagggcctggaacaactccaggaacagaccaagttcacacgcagagagctgcaggtcctgtaccgaggcttcaagaacgaatgccccagtgggattgtcaacgaggagaacttcaagcagatttattctcagttctttccccaaggagactccagcaactatgctacttttctcttcaatgcctttgacaccaaccacgatggctctgtcagttttgaggactttgtggctggtttgtcggtgattcttcgggggaccatagatgatagactgagctgggctttcaacttatatgacctcaacaaggacggctgtatcacaaaggaggaaatgcttgacattatgaagtccatctatgacatgatgggcaagtacacataccctgccctccgggaggaggccccaagagaacacgtggagagcttcttccagaagatggacaggaacaaggacggcgtggtgaccatcgaggaattcatcgagtcttgtcaacaggacgagaacatcatgaggtccatgcagctctcaccccttctcaactgatacctagtgctgaggacacccctggtgtagggaccaagtggttctccaccttctagtcccactctagaaaccacattagacagaaggtctcctgctatggtgctttccccatccctaatctcttagattttcctcaagactcccttctcagagaacacgctctgtccatgtccccagctggcttctcagcctagcctttgagggccctgtggggaggcggggacaagaaagcagaaaagtcttggccccgagccagtggttaggtcctaggaattggctggagtggaggccagaaagcctgggcagatgatgagagcccagctgggctgtcactgcaggttccggggcctacagccctgggtcagcagagtatgagttcccagactttccagaaggtccttagcaatgtcccagaaattcaccgtacacttctcagtgtcttaggagggcccgggatccagatgtctggttcatccctgaatcctctccctccttcttgctcgtatggtgggagtggtggccaggggaagatgagtggtgtcccggatgatgcctgtcaaggtcccacctcccctccggctgttctcatgacagctgtttggttctccatgacccctatctagatgtagaggcatggagtgagtcagggatttcccgaacttgagttttaccactcctcctagtggctgccttaggggaatgggaagaacccagtgtgggggcacccattagaatctttgcccggctcctcacaatgccctagggtcccctagggtacccgctccctctgtttagtctacccagagatgctcctgagctcacctagagggtagggacggtaggctccaggtccaacctctccaggtcagcaccctgccatgctgctgctcctcattaacaaacctgcttgtctcctcctgcgccccttctcagtcagccagggtctgaggggaagggcctcccgtttccccatccgtcagacatggttgactgctttgcattttgggctcttctatctattttgtaaaataagacatcagatccaataaaacacacggctatgcacaaaaaaaaaaaaaaaaaaaaaaaaa

SEQ ID NO: 28 - protein, 252 aa, Simian sp.
MRGQGRKESLSESRDLDGSYDQLTGHPPGPSKKALKQRFLKLLPCCGPQALPSVSENSVEDEFELSTVCHRPEGLEQLQEQTKFTRRELQVLYRGFKNECPSGIVNEENFKQIYSQFFPQGDSSNYATFLFNAFDTNHDGSVSFEDFVAGLSVILRGTIDDRLSWAFNLYDLNKDGCITKEEMLDIMKSIYDMMGKYTYPALREEAPREHVESFFQKMDRNKDGVVTIEEFIESCQQDENIMRSMQLSPLLN

SEQ ID NO: 29 - DNA, 1904 bp, Rattus sp.
  CDS (1)..(675)
atgaaccactgccctcgcaggtgccggagcccgttggggcaggcagctcgatctctctaccagttggtaactgggtcgctgtcgccagacagcgtagaggatgagtttgaattatccacggtgtgtcaccgacctgagggcctggaacaactccaggaacagaccaagttcacacgcagagagctgcaggtcctgtaccgaggcttcaagaacgaatgccccagtgggattgtcaacgaggagaacttcaagcagatttattctcagttctttccccaaggagactccagcaactatgctacttttctcttcaatgcctttgacaccaaccacgatggctctgtcagttttgaggactttgtggctggtttgtcggtgattcttcgggggaccatagatgatagactgagctgggctttcaacttatatgacctcaacaaggacggctgtatcacaaaggaggaaatgcttgacattatgaagtccatctatgacatgatgggcaagtacacataccctgccctccgggaggaggccccaagagaacacgtggagagcttcttccagaagatggacaggaacaaggacggcgtggtgaccatcgaggaattcatcgagtcttgtcaacaggacgagaacatcatgaggtccatgcagctctttgataatgtcatctagctccccagggagaggggttagtgtgtcctagggtgaccaggctgtagtcctagtccagacgaacctaaccctctctctccaggcctgtcctcatcttacctgtaccctgggggctgtagggattcaatatcctggggcttcagtagtccagatccctgagctaagtcacaaaagtaggcaagagtaggcaagctaaatctgggggcttcccaacccccgacagctctcaccccttctcaactgatacctagtgctgaggacacccctggtgtagggaccaagtggttctccaccttctagtcccactctagaaaccacattagacagaaggtctcctgctatggtgctttccccatccctaatctcttagattttcctcaagactcccttctcagagaacacgctctgtccatgtccccagctggcttctcagcctagcctttgagggccctgtggggaggcggggacaagaaagcagaaaagtcttggccccgagctagtggttaggtcctaggaattggctggagtggaggccagaaagcctgggcagatgatgagagcccagctgggctgtcactgcaggttccagggcctacagccctgggtcagcagagtatgagttcccagactttccagaaggtccttagcaatgtcccagaaattcaccatacacttctcagtgtcccggatgatgcctgtcaaggtcccacctcccctccggctgttctcatgacagctgtttggttctccatgacccctatctagatgtagaggcatggagtgagtcagggatttcccgaacttgagttttaccactcctcctagtggctgccttaggggaatgggaagaacccagtgtgggggcacccattagaatctttgcccggttcctcacaatgccctagggtcccctagggtacccgctccctctgtttagtctacccagagatgctcctgagctcacctagagggtagggacggtaggctccaggtccaacctctccaggtcagcaccctgccatgctgctgctcctcattaacaaacctgcttgtctcctcctgcgccccttctcagtcagccagggtctgaggggaagggcctcccgtttccccatccgtcagacatggttgactgctttgcattttgggctcttctatctattttgtaaaataagacatcagatccaataaaacacacggctatgcacaaaaaaaaaaaaaaaaaa

SEQ ID NO: 30 - protein, 225 aa, Rattus sp.
MNHCPRRCRSPLGQAARSLYQLVTGSLSPDSVEDEFELSTVCHRPEGLEQLQEQTKFTRRELQVLYRGFKNECPSGIVNEENFKQIYSQFFPQGDSSNYATFLFNAFDTNHDGSVSFEDFVAGLSVILRGTIDDRLSWAFNLYDLNKDGCITKEEMLDIMKSIYDMMGKYTYPALREEAPREHVESFFQKMDRNKDGVVTIEEFIESCQQDENIMRSMQLFDNVI

SEQ ID NO: 31 - DNA, 2841 bp, Homo sapiens
  CDS (1)..(768)
atgcagccggctaaggaagtgacaaaggcgtcggacggcagcctcctgggggacctcgggcacacaccacttagcaagaaggagggtatcaagtggcagaggccgaggctcagccgccaggctttgatgagatgctgcctggtcaagtggatcctgtccagcacagccccacagggctcagatagcagcgacagtgagctggagctgtccacggtgcgccaccagccagaggggctggaccagctgcaggcccagaccaagttcaccaagaaggagctgcagtctctctacaggggctttaagaatgagtgtcccacgggcctggtggacgaagacaccttcaaactcatttacgcgcagttcttccctcagggagatgccaccacctatgcacacttcctcttcaacgcctttgatgcggacgggaacggggccatccactttgaggactttgtggttggcctctccatcctgctgcggggcacagtccacgagaagctcaagtgggcctttaatctctacgacattaacaaggatggctacatcaccaaagaggagatgctggccatcatgaagtccatctatgacatgatgggccgccacacctaccccatcctgcgggaggacgcgccggcggagcacgtggagaggttcttcgagaaaatggaccggaaccaggatggggtagtgaccattgaagagttcctggaggcctgtcagaaggatgagaacatcatgagctccatgcagctgtttgagaatgtcatctaggacacgtccaaaggagtgcatggccacagccacctccacccccaagaaacctccatcctgccaggagcagcctccaagaaacttttaaaaaatagatttgcaaaaagtgaacagattgctacacacacacacacacacacacacacacacacacacacacagccattcatctgggctggcagaggggacagagttcagggaggggctgagtctggctaggggccgagtccaggagccccagccagcccttcccaggccagcgaggcgaggctgcctctgggtgagtggctgacagagcaggtctgcaggccaccagctgctggatgtcaccaagaaggggctcgagtgcccctgcaggggagggtccaatctccggtgtgagcccacctcgtcccgttctccattctgctttcttgccacacagtgggccggccccaggctcccctggtctcctccccgtagccactctctgcccactacctatgcttctagaaagcccctcacctcaggaccccagagggaccagctggggggcaggggggagagggggtaatggaggccaagcctgcagctttctggaaattcttccctgggggtcccaggatcccctgctactccactgacctggaagagctgggtaccaggccacccactgtggggcaagcctgagtggtgaggggccactgggccccattctccctccatggcaggaaggcgggggatttcaagtttagggattgggtcgtggtggagaatctgagggcactctctgccagctccacagggtgggatgagcctctccttgccccagtcctggttcagtgggaatgcagtgggtggggctgtacacaccctccagcacagactgttccctccaaggtcctcttaggtcccgggaggaacgtggttcagagactggcagccagggagcccggggcagagctcagaggagtctgggaaggggcgtgtccctcctcttcctgtagtgcccctcccatggcccagcagcttggctgagccccctctcctgaagcagtgtcgccgtccctctgccttgcacaaaaagcacaagcattccttagcagctcaggcgcagccctagtgggagcccagcacactgcttctcggaggccaggccctcctgctggctgaggcttgggcccagtagccccaatatggtggccctggggaagaggccttgggggtctgctctgtgcctgggatcagtggggccccaaagcccagcccggctgaccaacattcaaaagcacaaaccctggggactctgcttggctgtcccctccatctggggatggagaatgccagcccaaagctggagccaatggtgagggctgagagggctgtggctgggtggtcagcagaaacccccaggaggagagagatgctgctcccgcctgattggggcctcacccagaaggaacccggtcccaggccgcatggcccctccaggaacattcccacataatacattccatcacagccagcccagctccactcagggctggcccggggagtccccgtgtgccccaagaggctagccccagggtgagcagggccctcagaggaaaggcagtatggcggaggccatgggggcccctcggcattcacacacagcctggcctcccctgcggagctgcatggacgcctggctccaggctccaggctgactgggggcctctgcctccaggagggcatcagctttccctggctcagggatcttctccctcccctcacccgctgcccagccctcccagctggtgtcactctgcctctaaggccaaggcctcaggagagcatcaccaccacacccctgccggccttggccttggggccagactggctgcacagcccaaccaggaggggtctgcctcccacgctgggacacagaccggccgcatgtctgcatggcagaagcgtctcccttggccacggcctgggagggtggttcctgttctcagcatccactaatattcagtcctgtatattttaataaaataaacttgacaaaggaaaaaaaaaaaaaaaaaattcctgcggccgcgttctcca

SEQ ID NO: 32 - protein, 256 aa, Homo sapiens
MQPAKEVTKASDGSLLGDLGHTPLSKKEGIKWQRPRLSRQALMRCCLVKWILSSTAPQGSDSSDSELELSTVRHQPEGLDQLQAQTKFTKKELQSLYRGFKNECPTGLVDEDTFKLIYAQFFPQGDATTYAHFLFNAFDADGNGAIHFEDFVVGLSILLRGTVHEKLKWAFNLYDINKDGYITKEEMLAIMKSIYDMMGRHTYPILREDAPAEHVERFFEKMDRNQDGVVTIEEFLEACQKDENIMSSMQLFENVI

SEQ ID NO: 33 - DNA, 442 bp, Rattus sp.
  CDS (1)..(327)
tttgaggactttgtggttgggctctccatcctgcttcgagggaccgtccatgagaagctcaagtgggccttcaatctctacgacatcaacaaggacggttacatcaccaaagaggagatgctggccatcatgaagtccatctacgacatgatgggccgccacacctaccctatcctgcgggaggacgcacctctggagcatgtggagaggttcttccagaaaatggacaggaaccaggatggagtagtgactattgatgaatttctggagacttgtcagaaggacgagaacatcatgagctccatgcagctgtttgagaacgtcatctaggacatgtaggaggggaccctgggtggccatgggttctcaacccagagaagcctcaatcctgacaggagaagcctctatgagaaacatttttctaatatatttgcaaaaagtg

SEQ ID NO: 34 - protein, 109 aa, Rattus sp.
FEDFVVGLSILLRGTVHEKLKWAFNLYDINKDGYITKEEMLAIMKSIYDMMGRHTYPILREDAPLEHVERFFQKMDRNQDGVVTIDEFLETCQKDENIMSSMQLFENVI

SEQ ID NO: 35 - DNA, 2644 bp, Mus musculus
  CDS (49)..(816)
cgggctgcaaagcgggaagattagtgacggtccctttcagcagcagagatgcagaggaccaaggaagccgtgaaggcatcagatggcaacctcctgggagatcctgggcgcataccactgagcaagagggaaagcatcaagtggcaaaggccacggttcacccgccaggccctgatgcgttgctgcttaatcaagtggatcctgtccagtgctgccccacaaggctcagacagcagtgacagtgaactggagttatccacggtgcgccatcagccagagggcttggaccagctacaagctcagaccaagttcaccaagaaggagctgcagtccctttaccgaggcttcaagaatgagtgtcccacaggcctggtggatgaagacaccttcaaactcatttattcccagttcttccctcagggagatgccaccacctatgcacacttcctcttcaatgcctttgatgctgatgggaacggggccatccactttgaggactttgtggttgggctctccatcctgcttcgagggacggtccatgagaagctcaagtgggccttcaatctctatgacattaacaaggatggttgcatcaccaaggaggagatgctggccatcatgaagtccatctacgacatgatgggccgccacacctaccccatcctgcgggaggatgcacccctggagcatgtggagaggttctttcagaaaatggacaggaaccaggatggagtggtgaccattgatgaatttctggagacttgtcagaaggatgagaacatcatgaactccatgcagctgtttgagaacgtcatctaggacatgtgggaggggaccccagtggtcattgcttctcaacccagagaagcctcaatcctgacaggagaagcctctatgagaaacatttttctaatatatttgcaaaaagtgagcagtttacttccaagacacagccaccgtcacacacagacacagacatacagacacacacacacacacacacacatggttcctctggcttggccaaggaagtggcagccagaaggcacccccgcctattcctaggtcaataaaaaaggctgcctctgggatggccagccctggctagatgttacccacaaggaactcagagatcgagaggaccaggtctacaaagctaaggtccctgtgtcttttctaccactcgggagatcaaactactccctgcctatggacccatgctcttaggaagctcccagaaactccaaggggacaaagaggggagaggtctataggaagaaatggttttggaagctgggcttgcagccttatgctaatgatcacctggggtcctggaacccgagtgccaggctacctactatgccgtgagcttagatagtgaggggccattggactaagacctcctgtaagagtggggcaggattgaggtttttggagaaactgaggaaacaatttgtccataccactgggtgaagactgctggccagtgggaatgtggctggtggagatttcccaacttccagcaccaggatggcctctccaaggtcctctttgattccctggggagatcacctggctcatagactgacaaccagggaactgggctgaaatgggaggtctggtagggggcatccccctccttttccctggccacttgccacccagttccttaacacagtggatcggccacacctctgtggctgcccttgaacagactcatcccgaccaagacaaaaaagcacaaactcctagcagctcaggccaagcccacaagggaaggcctgggtccctgcagccctgattcagtggccgaggaagacgctcagacatccatcctgtacctcggagccttgggggtctcacagccctttcccagcccagctcgccaacattctaaagcacaaacctgcggattctgcttgcttgggctgcgccctggggattgaaggccactgttaaccctaagctggagctagccctgagggctggggacctgtgaccaggcaacaggtcagcagaccctcaggaggagagagagctgttcctgcctccccaggcctcgcccagaaggaacagtgtcccaagaagcatgtttcctggaggaacatccccacaaaagtacattccatcatctgaagcccggtctctgctcaggcctgcctctgaaagtccacgtgtgttccccagaaggccagccccaagataagggaggtccttagaggaaggacagggtgacaacacccctatacacaggtggaccccccctctgaggactgtactgaccccatctccatcctgaccggggccttcctttacccgatctacagaccaccagttctccctggctcagggaccccctgtcccccagtctgactcttcccatcgaggtccctgtcttgtgaaaagccaaggccacgggaaaaggccaccactctaacctgctgcatcccttagcctctggctgcacgcccaacctggaggggtctgtcccctttgcagggacacagactggccgcatgtccgcatggcagaagcgtctcccttgggtgcagcctggaagggtggtttctgtctcagcgcccaccaatattcagtcctatatattttaataaaagaaacttgacaaaggaaaaaaaaaaaaaaa

SEQ ID NO: 36 - protein, 256 aa, Mus musculus
MQRTKEAVKASDGNLLGDPGRIPLSKRESIKWQRPRFTRQALMRCCLIKWILSSAAPQGSDSSDSELELSTVRHQPEGLDQLQAQTKFTKKELQSLYRGFKNECPTGLVDEDTFKLIYSQFFPQGDATTYAHFLFNAFDADGNGAIHFEDFVVGLSILLRGTVHEKLKWAFNLYDINKDGCITKEEMLAIMKSIYDMMGRHTYPILREDAPLEHVERFFQKMDRNQDGVVTIDEFLETCQKDENIMNSMQLFENVI

SEQ ID NO: 37 - DNA, 531 bp, Homo sapiens
  CDS (1)..(336)
  At position 495, n=any amino acid
cacgaggtggaaagcatttcggctcagctggaggaggccagctctacaggcggtttcctgtacgctcagaacagcaccaagcgcagcattaaagagcggctcatgaagctcttgccctgctcagctgccaaaacgtcgtctcctgctattcaaaacagcgtggaagatgaactggagatggccaccgtcaggcatcggcccgaagcccttgagcttctggaagcccagagcaaatttaccaagaaagagcttcagatcctttacagaggatttaagaacgtaagaactttctttttgactttaccttcacacaattcccagaggagcattgagaaatgagaggaaaagggggaaaatatcccattctatgagaagccccatcatatgtatatttcatactgatccttcccagataggaatataatcagtatctgtggactttgaatctctgtggcacacccatgctggcatactgtaattgcccattaaacaaanagtttttgagaaaaaaaaaaaaaaaaaaaaaaaaaa

SEQ ID NO: 38 - protein, 112 aa, Homo sapiens
HEVESISAQLEEASSTGGFLYAQNSTKRSIKERLMKLLPCSAAKTSSPAIQNSVEDELEMATVRHRPEALELLEAQSKFTKKELQILYRGFKNVRTFFLTLPSHNSQRSIEK

SEQ ID NO: 39 - DNA, 2176 bp, Homo sapiens
  CDS (2)..(124)
tgaaaggttcttcgagaaaatggaccggaaccaggatggggtagtgaccattgaagagttcctggaggcctgtcagaaggatgagaacatcatgagctccatgcagctgtttgagaatgtcatctaggacacgtccaaaggagtgcatggccacagccacctccacccccaagaaacctccatcctgccaggagcagcctccaagaaacttttaaaaaatagatttgcaaaaagtgaacagattgctacacacacacacacacacacacacacacacacacacacacagccattcatctgggctggcagaggggacagagttcagggaggggctgagtctggctaggggccgagtccaggagccccagccagcccttcccaggccagcgaggcgaggctgcctctgggtgagtggctgacagagcaggtctgcaggccaccagctgctggatgtcaccaagaaggggctcgagtgcccctgcaggggagggtccaatctccggtgtgagcccacctcgtcccgttctccattctgctttcttgccacacagtgggccggccccaggctcccctggtctcctccccgtagccactctctgcccactacctatgcttctagaaagcccctcacctcaggaccccagagggaccagctggggggcaggggggagagggggtaatggaggccaagcctgcagctttctggaaattcttccctgggggtcccaggatcccctgctactccactgacctggaagagctgggtaccaggccacccactgtggggcaagcctgagtggtgaggggccactgggccccattctccctccatggcaggaaggcgggggatttcaagtttagggattgggtcgtggtggagaatctgagggcactctctgccagctccacagggtgggatgagcctctccttgccccagtcctggttcagtgggaatgcagtgggtggggctgtacacaccctccagcacagactgttccctccaaggtcctcttaggtcccgggaggaacgtggttcagagactggcagccagggagcccggggcagagctcagaggagtctgggaaggggcgtgtccctcctcttcctgtagtgcccctcccatggcccagcagcttggctgagccccctctcctgaagcagtgtcgccgtccctctgccttgcacaaaaagcacaagcattccttagcagctcaggcgcagccctagtgggagcccagcacactgcttctcggaggccaggccctcctgctggctgaggcttgggcccagtagccccaatatggtggccctggggaagaggccttgggggtctgctctgtgcctgggatcagtggggccccaaagcccagcccggctgaccaacattcaaaagcacaaaccctggggactctgcttggctgtcccctccatctggggatggagaatgccagcccaaagctggagccaatggtgagggctgagagggctgtggctgggtggtcagcagaaacccccaggaggagagagatgctgctcccgcctgattggggcctcacccagaaggaacccggtcccaggccgcatggcccctccaggaacattcccacataatacattccatcacagccagcccagctccactcagggctggcccggggagtccccgtgtgccccaagaggctagccccagggtgagcagggccctcagaggaaaggcagtatggcggaggccatgggggcccctcggcattcacacacagcctggcctcccctgcggagctgcatggacgcctggctccaggctccaggctgactgggggcctctgcctccaggagggcatcagctttccctggctcagggatcttctccctcccctcacccgctgcccagccctcccagctggtgtcactctgcctctaaggccaaggcctcaggagagcatcaccaccacacccctgccggccttggccttggggccagactggctgcacagcccaaccaggaggggtctgcctcccacgctgggacacagaccggccgcatgtctgcatggcagaagcgtctcccttggccacggcctgggagggtggttcctgttctcagcatccactaatattcagtcctgtatattttaataaaataaacttgacaaaggaaaaaaaaaaaaaaaaaa

SEQ ID NO: 40 - protein, 41 aa, Homo sapiens
ERFFEKMDRNQDGVVTIEEFLEACQKDENIMSSMQLFENVI

SEQ ID NO: 41 - DNA, 2057 bp, Rattus sp.; identical to SEQ ID NO: 27
  CDS (208)..(963)

SEQ ID NO: 42 - protein, 252 aa, Rattus sp.; identical to SEQ ID NO: 28

SEQ ID NO: 43 - protein, 26 aa, Artificial Sequence
  Xaas at positions 2,5,6,9,17,25 and 26 may be Ile, Leu, Val or Met
EXXXXXXXXDKDGDGXXXXXEFXXXX

SEQ ID NO: 44 - DNA, 40 bp, Rattus sp.
taatacgactcactatagggactggccatcctgctctcag

SEQ ID NO: 45 - DNA, 40 bp, Rattus sp.
attaaccctcactaaagggacactactgtttaagctcaag

SEQ ID NO: 46 - DNA, 40 bp, Rattus sp.
taatacgactcactatagggcacctcccctccggctgttc

SEQ ID NO: 47 - DNA, 40 bp, Rattus sp.
attaaccctcactaaagggagagcagcagcatggcagggt

SEQ ID NO: 48 - DNA, 2413 bp, Simian sp.
  CDS (265)..(963)
gtcgacccacgcgtccggtgcgctgtggttgcgggggggagccccgccagccaaatgccaggatcagcatgagaggctggactttagtccaggtctgtcctcaccccgggggaccgccggctttgcagggtgcagctgcgaggaactgctcacttttttccccttgcaagtctttgttccaagcctgacgttgctacgattctgtaattaactccctccactccaaaggggtctggaggctgggatgctctgccagctcagaggatgttgactctggagtgggagtccgaaggactgcaaacagtgggtattgttgtgattatatgtgcatctctgaagctgcttcatttgctgggactgattgatttttcggaagacagcgtggaagatgaactggagatggccactgtcaggcatcggcctgaggcccttgagcttctggaagcccagagcaaatttaccaagaaagagcttcagatcctttacagaggatttaagaacgaatgccccagtggtgttgttaatgaagaaaccttcaaagagatttactcgcagttctttccacagggagactctacaacatatgcacattttctgttcaatgcgtttgatacggaccacaatggagctgtgagtttcgaggatttcatcaaaggtctttccattttgctccgggggacagtacaagaaaaactcaattgggcatttaatctgtatgatataaataaagatggctacatcactaaagaggaaatgcttgatataatgaaagcaatatacgacatgatgggtaaatgtacatatcctgtcctcaaagaagatgcacccagacaacacgtcgaaacattttttcagaaaatggacaaaaataaagatggggttgttaccatagatgagttcattgaaagctgccaaaaagatgaaaacataatgcgctccatgcagctctttgaaaatgtgatttaacttgtcaactagatcctgaatccaacagacaaatgtgaactattctaccacccttaaagtcggagctaccacttttagcatagattgctcagcttgacactgaagcatattatgcaaacaagctttgttttaatataaagcaatccccaaaagatttgagtttctcagttataaatttgcatcctttccataatgccactgagttcatgggatgttctaactcatttcatactctgtgaatattcaaaagtaatagaatctggcatatagttttattgattccttagccatgggattattgaggctttcacatatcagtgattttaaaataccagtgttttttgctctcatttgtatgtattcagtcctaggattttgaatggttttctaatatactgacatctgcatttaatttccagaaattaaattaattttcatgtctgaatgctgtaattccatttatatactttaagtaaacaaataagattactacaattaaacacatagttccagtttctatggccttcccttcccaccttctattataaattaattttatctggtatttttaaacatttaaaaatttatcatcagatatcagcatatgcctaattatgcctaatgaaacttaataagcatttaattttccatcatacattatagccaaggcctatatactatatataattttggatttgtttaatcttacaggctgttttccattgtatcatcaagtggaagttcaagacggcatcaaacaaaacaaggatgtttacagacatatgcaaagggtcaggatatctatcctccagtatatgttaatgcttaataacaagtaatcctaacagcattaaaggccaaatctgtcctctttcccctgacttccttacagcatgtttatattacaagccattcagggacaaagaaaccttgactaccccactgtctactaggaacaaacaaacagcaagcaaaattcactttgaaagcaccagtggttccattacattgacaactactaccaagattcagtagaaaataagtgctcaacaactaatccagattacaatatgatttagtgcatcataaaattccaacaattcagattatttttaatcatctcagccacaactgtaaagttgccacattactaaagacacacacatcgtccctgttttgtagaaatatcacaaagaccaagaggctacagaaggaggaaatttgcaactgtctttgcaacaataaatcaggtatctattctggtgtagagataggatgttgaaagctgccctgctatcaccagtgtagaaattaagagtagtacaatacatgtacactgaaatttgccatcgcgtgtttgtgtaaactcaatgtgcacattttgtatttcaaaaagaaaaaataaaagcaaaataaaatgttwawaamwmwaaaaaaaaaaaaaaaa

SEQ ID NO: 49 - protein, 233 aa, Simian sp.
MLTLEWESEGLQTVGIVVIICASLKLLHLLGLIDFSEDSVEDELEMATVRHRPEALELLEAQSKFTKKELQILYRGFKNECPSGVVNEETFKEIYSQFFPQGDSTTYAHFLFNAFDTDHNGAVSFEDFIKGLSILLRGTVQEKLNWAFNLYDINKDGYITKEEMLDIMKAIYDMMGKCTYPVLKEDAPRQHVETFFQKMDKNKDGVVTIDEFIESCQKDENIMRSMQLFENVI

SEQ ID NO: 50 - DNA, 1591 bp, Simian sp.
  CDS (265)..(963)
gtcgacccacgcgtccggtgcgctgtggttgcgggggggagccccgccagccaaatgccaggatcagcatgagaggctggactttagtccaggtctgtcctcaccccgggggaccgccggctttgcagggtgcagctgcgaggaactgctcacttttttccccttgcaagtctttgttccaagcctgacgttgctacgattctgtaattaactccctccactccaaaggggtctggaggctgggatgctctgccagctcagaggatgttgactctggagtgggagtccgaaggactgcaaacagtgggtattgttgtgattatatgtgcatctctgaagctgcttcatttgctgggactgattgatttttcggaagacagcgtggaagatgaactggagatggccactgtcaggcatcggcctgaggcccttgagcttctggaagcccagagcaaatttaccaagaaagagcttcagatcctttacagaggatttaagaacgaatgccccagtggtgttgttaatgaagaaaccttcaaagagatttactcgcagttctttccacagggagactctacaacatatgcacattttctgttcaatgcgtttgatacggaccacaatggagctgtgagtttcgaggatttcatcaaaggtctttccattttgctccgggggacagtacaagaaaaactcaattgggcatttaatctgtatgatataaataaagatggctacatcactaaagaggaaatgcttgatataatgaaagcaatatacgacatgatgggtaaatgtacatatcctgtcctcaaagaagatgcacccagacaacacgtcgaaacattttttcaggctgttttccattgtatcatcaagtggaagttcaagacggcatcaaacaaaacaaggatgtttacagacatatgcaaagggtcaggatatctatcctccagtatatgttaatgcttaataacaagtaatcctaacagcattaaaggccaaatctgtcctctttcccctgacttccttacagcatgtttatattacaagccattcagggacaaagaaaccttgactaccccactgtctactaggaacaaacaaacagcaagcaaaattcactttgaaagcaccagtggttccattacattgacaactactaccaagattcagtagaaaataagtgctcaacaactaatccagattacaatatgatttagtgcatcataaaattccaacaattcagattatttttaatcatctcagccacaactgtaaagttgccacattactaaagacacacacatcgtccctgttttgtagaaatatcacaaagaccaagaggctacagaaggaggaaatttgcaactgtctttgcaacaataaatcaggtatctattctggtgtagagataggatgttgaaagctgccctgctatcaccagtgtagaaattaagagtagtacaatacatgtacactgaaatttgccatcgcgtgtttgtgtaaactcaatgtgcacattttgtatttcaaaaagaaaaaataaaagcaaaataaaatgttwawaamwmwaaaaaaaaaaaaaaaa

SEQ ID NO: 51 - protein, 233 aa, Simian sp.
MLTLEWESEGLQTVGIVVIICASLKLLHLLGLIDFSEDSVEDELEMATVRHRPEALELLEAQSKFTKKELQILYRGFKNECPSGVVNEETFKEIYSQFFPQGDSTTYAHFLFNAFDTDHNGAVSFEDFIKGLSILLRGTVQEKLNWAFNLYDINKDGYITKEEMLDIMKAIYDMMGKCTYPVLKEDAPRQHVETFFQAVFHCIIKWKFKTASNKTRMFTDICKGSGYLSSSIC

SEQ ID NO: 52 - DNA, 2051 bp, Rattus sp.
  CDS (85)..(1305)
ggtggagctaagcactcactgcggtgctgccctgcgtctgcagagaacaaggaaagcttctctgcagggctgtcagctgccaaaatgaacggcgtggaagggaacaacgagctccctctcgctaacacctcgacctccgcccttgtcccggaagatctggatctgaagcaagaccagccgctcagcgaggaaactgacacggtgcgggagatggaggctgcaggtgaggccggtgcggagggaggcgcgtcccccgattcggagcactgcgacccccagctctgcctccgagtggctgagaatggctgtgctgccgcagcgggagaggggctggaggatggtctgtcttcatcaaagtgtggggacgcacccttggcgtctgtggcagccaacgacagcaataaaaatggctgtcagcttgcagggccgctcagccctgctaagccaaaaactctggaagccagtggtgcagtgggcctggggtcgcagatgatgccagggccgaagaagaccaaggtaatgactaccaagggcgccatctctgcgactacaggcaaggaaggagaagcaggggcggcaatgcaggaaaagaagggggtgcagaaagaaaaaaaggcagctggaggagggaaagacgagactcgtcctagagcccctaagatcaataactgcatggactccctggaagccatcgatcaagagctgtcaaatgtaaatgcgcaagctgacagggccttcctccagctggaacgcaaatttgggcggatgagaaggctccacatgcagcgccgaagtttcatcatccaaaacatcccaggtttctgggtcacagcgtttcggaaccacccgcaactgtcaccgatgatcagtggccaagatgaagacatgatgaggtacatgatcaatttagaggtggaggagcttaagcacccaagagcagggtgcaaatttaagttcatcttccaaagcaacccctacttccgaaatgaggggctggtcaaagagtacgagcgcagatcctcaggtcgagtggtgtcgctctctacgccaatccgctggcaccggggtcaagaaccccaggcccatatccacaggaatagagaggggaacacgattcccagtttcttcaattggttctcagaccacagcctcctagaattcgacagaatagctgaaattatcaaaggggagctttggtccaatcccctacaatactacctgatgggcgatgggccacgcagaggagttcgagtcccaccaaggcagccagtggagagtcccaggtccttcaggttccagtctggctaagctctgccctcgtgagaagctcttacagaagagtccttaccaccttctcagcttggctagcagcatgcagccttctgtctgctttctcttccttggattgtgtcctttggttcttctaagtctccggtagtttcaaggttgtggcttccaagtctttgctcttctttctcttggccatcacgatgtcctgcatagtgttaatggtgttccaagtgcatggcctccaaactgcttctatgccaagctcacgtgctgtagtttgtactgcttttctttgcatggcttggttcctgtctgtgatcttctaggttttttgttttcttttttaaaagtggttctctatcaaaagaaagcttgacatatccttaccaagaactagccagatttcatactgtgttcccgatatctatgtactgtgaagaactgtgagtttcgccactgcaagatgggactgtatcccaatccagccatcagcccaacaggacattccaagctgtcaccaactgatcctagctgtcttcctgggcctttgccatttaccctgctttttatctatagaatgagcaggtggctggtaggtgactactaggtaagagtgaagtattaggtgaggagtgttttctgtcaccacattgttcttgtaccaatgcatcatgatcagcttggatcagctactgactgtctgatatttctaacccccaacacaaaaaaaaaaaaaaaaaaaaaaaaaaaaaaaaaaaaaa

SEQ ID NO: 53 - protein, 407 aa, Rattus sp.
MNGVEGNNELPLANTSTSALVPEDLDLKQDQPLSEETDTVREMEAAGEAGAEGGASPDSEHCDPQLCLRVAENGCAAAAGEGLEDGLSSSKCGDAPLASVAANDSNKNGCQLAGPLSPAKPKTLEASGAVGLGSQMMPGPKKTKVMTTKGAISATTGKEGEAGAAMQEKKGVQKEKKAAGGGKDETRPRAPKINNCMDSLEAIDQELSNVNAQADRAFLQLERKFGRMRRLHMQRRSFIIQNIPGFWVTAFRNHPQLSPMISGQDEDMMRYMINLEVEELKHPRAGCKFKFIFQSNPYFRNEGLVKEYERRSSGRVVSLSTPIRWHRGQEPQAHIHRNREGNTIPSFFNWFSDHSLLEFDRIAEIIKGELWSNPLQYYLMGDGPRRGVRVPPRQPVESPRSFRFQSG

SEQ ID NO: 54 - DNA, 4148 bp, Homo sapiens
  CDS (88)..(1329)
ggggtggtgctagacgtttcgggcagagctcggccgctgcggaggacaaggaactctccctctcccactagtctgacttcttccaaaatgagcggcctggatgggggcaacaagctccctctcgcccaaaccggcggcctggctgctcccgaccatgcctcaggagatccggacctagaccagtgccaagggctccgtgaagaaaccgaggcgacacaggtgatggcgaacacaggtgggggcagcctggagaccgttgcggaggggggtgcatcccaggatcctgtcgactgtggccccgcgctccgcgtcccagttgccgggagtcgcggcggtgcagcgaccaaagccgggcaggaggatgctccaccttctacgaaaggtctggaagcagcctctgccgccgaggctgctgacagcagccagaaaaatggctgtcagcttggagagccccgtggccctgctgggcagaaggctctagaagcctgtggcgcagggggcttggggtctcagatgataccggggaagaaggccaaggaagtgacgactaaaaaacgcgccatctcggcagcagtggaaaaggagggagaagcaggggcggcgatggaggaaaagaaggtagtgcagaaggaaaaaaaggtggcaggaggggtgaaagaggagacacggcccagggccccgaagatcaataactgcatggactcactggaggccatcgatcaagagttgtcaaacgtaaatgcccaggctgacagggccttccttcagcttgagcgcaagtttggccgcatgcgaaggctccacatgcagcgcagaagtttcattatccagaatatcccaggtttctgggttactgcctttcgaaaccacccccagctgtcacctatgatcagtggccaagatgaagacatgctgaggtacatgatcaatttggaggtggaggagcttaaacaccccagagcaggctgcaaattcaagttcatctttcagggcaacccctacttccgaaatgaggggcttgtcaaggaatatgaacgcagatcctctggccgggtggtgtctctttccactccaatccgctggcaccgaggccaagacccccaggctcatatccacagaaaccgggaagggaacactatccctagtttcttcaactggttttcagaccacagccttctagaattcgacagaattgcagagattatcaaaggagaactgtggcccaatcccctacaatactacctgatgggtgaagggccccgtagaggaattcgaggcccaccaaggcagccagtggagagcgccagatccttcaggttccagtctggctaatctctgtcctgtgagaagcttctgcacaagtttccttaccacctcctcttggacctatgcttggccaacagcatgcagtcttccatctgctttctcttcatactgtggattatcttttcctttggttctaaatcttcagtaatcggttgcaagattgttggcttacctgcctgtgccattcttcctctgggccttcatgcttttctgcattgtgttaacatgtttcaagtgcatggccttctacggcttctatgccaagcgtatgatactatagatatagtgtaccatactgcctttctttgcatggcttggaccctatctgtgaccatgctcttctcccaatttaagtggttctgtaccacaaagaatcttgatacattttcacaaataactgattgggcttcatactttatgctggctgtgtcctgatacccatgtacttatggtaagctatttgggtattaccactgcaagacaaaactgatatcttaacccggccatcaacccaaattggacattccagactaccaccaactggatcccagctgccttcctgggcttgtgccatccaccctactggttatctgatagaacaagctggtggctgatgggtgactgctaggcgtgactgaggtaatagatgaaaagtgttctatgttatcacattggttttcctgtacctttggttactctacgtcatgaccagctgctggtgagtatgaagcctgtgctatagcccacccctactcactctcaccttctggttgaactttgcttaggccaccattgtctgcctcatcaggaactatctgtagacgtagctcccagggagctcacagcaacaccccctaccaccaggatgggcagtaatatgtgacagagcccaaagcaaggctggaacgcagtcccttccagcttagtctttctgactcctagccaacaaaccatccttaatgtgagcaacttctttaggcatttcctcttttccccgcctgcacccactctgaacatgacaaaagttgccagagttggggcattgaggaagagatatttctggaatgtgagacttgttatgcctctgtctctttctctccctccccctcccctctccctccccctctccctcccatcccttttcttccctttcactctgaagcagttttagcttattaacagaaaacaaaactggcaaagcaggctttttgtttaatttgctctttccctgattgtgttcagagagaaaggttatgattaaatgggctccagatctcttattgcccttattcctccaccccacttcttttagcaaggtctgaaagtttcaaagggagacctataggttaattgtttagttataggcagtgttaaattaggcagattttgacatatttatctttttaccccatccattctaccaaaacctgtgtatttcttgagtttttagtttgagaagctggaaagagagagaagggcctcacagtgatgggttcaggacgggtcaaaggcaaaggcctttgtgatgtgagcaaaggcaaccaaaacttagcctcactccacttttctaaagatggaaattcttttttgggccttggactgcttctagggtagcattttgtaggtcactcttctcctttgtactattttgtttctgccctgatgtcccttgggtctccatcctactgcctggctttcttggccctcatttctcagcttctgcatttccttccctgctcctaacaaatgaagaagcaggctgcagcctgcattgtggaagatctccagcctccttgtaggggataaggggatgtgtagcatctgtgtggattttcacggacaagttccagtaggtgggacagtgatgccgtcaaggcttagttatgatcatgtgtggtgataaagaccatccaccatcacccttttcccctttggttttgaaggccttgccctaagctacctgagggtttaggaggtctgaacacacacagtggagaggttaatctaggttgggaaactgagtaaaagtccagagcaggaatgagcctgctgtggcgtgggtttggaaaggctcacaggaaagaacctgcaggatcaggggtgggaggggaggcccctgaggtgctctccagggaagaggggctggggtttaaatagcatgcttggaggaagattttccttcaatttttcctaagtccttgaattcaccagtagatttttgtaaacaaaatgtaagtcgatgttttctctcaattatcctaggagtgacctttatatgtgtggaagattaatggtatatgctccttatgtcactgtttttgagtaaaatccatttcctttctctgtttcagcctatgacaaaattgatgtttacaggcctgctttttgcttataattgacaacatgtgcaaaaataccaaatttgtgtcctgtgcagtatgaagaattcagtgaatattcattaatgtattagcttgttttgctctctgttcatatatggctctattcttagaaatataatttgaatgtgatctttcaatagtctgaatattttacaaattatagctatgtcttgtgaaaataacctcaaaaagaaaaatacgactctgttgtcttacttgatatttcttgccctagtaatgtacttgacatttatgttcctaagcagtgtaagtaccagtagaatttctctgtcaaactcaatgatcatttagtacttttgtcttctcccatgtgcttgaaggaaaaataaagtgtcactaccgtatttcttgttttcatcaaaaaataaaaataatttaaaaaacaaaaaaaaaaaaaaa

SEQ ID NO: 55 - protein, 414 aa, Homo sapiens
MSGLDGGNKLPLAQTGGLAAPDHASGDPDLDQCQGLREETEATQVMANTGGGSLETVAEGGASQDPVDCGPALRVPVAGSRGGAATKAGQEDAPPSTKGLEAASAAEAADSSQKNGCQLGEPRGPAGQKALEACGAGGLGSQMIPGKKAKEVTTKKRAISAAVEKEGEAGAAMEEKKVVQKEKKVAGGVKEETRPRAPKINNCMDSLEAIDQELSNVNAQADRAFLQLERKFGRMRRLHMQRRSFIIQNIPGFWVTAFRNHPQLSPMISGQDEDMLRYMINLEVEELKHPRAGCKFKFIFQGNPYFRNEGLVKEYERRSSGRVVSLSTPIRWHRGQDPQAHIHRNREGNTIPSFFNWFSDHSLLEFDRIAEIIKGELWPNPLQYYLMGEGPRRGIRGPPRQPVESARSFRFQSG

SEQ ID NO: 56 - DNA, 2643 bp, Rattus sp.
  CDS (1)..(801)
ctgaaaggggcgaggcccagggtggtgaactccacctgcagtgacttcaaccatggctcagctctgcacatcgctgcctcgaatctgtgcctgggcgccgccaaatgtttactggagcatggtgccaacccagcgctgaggaatcgaaaaggacaggtaccagcggaagtggtcccagaccccatggacatgtcccttgacaaggcagaggcagccctggtggccaaggaattgcggacgctgctagaagaggctgtgccactgtcctgcacccttcctaaagtcacactacccaactatgacaacgtcccaggcaatctcatgctcagcgcgctgggcctgcgtctaggagaccgagtgctcctcgatggccagaagacgggcacgctgaggttctgcgggaccaccgagttcgccagtggccagtgggtgggcgtggagctagatgaaccggaaggcaagaacgacggcagcgttgggggtgtccggtacttcatctgccctcccaagcagggtctctttgcatctgtgtccaaggtctccaaggcagtggatgcacccccctcatctgttacctccacgccccgcactccccggatggacttctcccgtgtaacgggcaaaggccggagggaacacaaagggaagaagaagtccccatcttccccatctctgggcagcctgcagcagcgtgaaggggccaaagctgaagttggagaccaagtccttgtggcaggccagaacagggattgtgcgtttctatgggaagacagactttgctccaggttactggtatggcattgaactggaccagcccacgggcaagcatgacggctctgtgttcggtgtccggtactttacctgtgccccgaggcacggggtctttgcaccagcatctcgtatccagaggattggtggatccactgatccccctggagacagtgttggagcaaaaaaagtgcatcaagtgacaatgacacagcccaaacgcaccttcacaacagtccggaccccaaaggacattgcatcagagaactctatctccaggttactcttctgctgctggtttccttggatgctgagggcggagatgcagtcttagagacctggatacctgacacagagacagagtcccctctagcatctcctgacacaaggagaccccagtcaccctaagatagagattcccagtgacacctccagaatagaaaccccgttagccagccctcgattactgaggtcccattattaacagatctcccatgacgactcccccaaatacagacctcatgttaccccaaaagagattccctgagtagcaccttcaggctagtccctgtcccctacccctcagagcagatttcccccaataaacattttccacatcacccaagggatgctgaccctctccacgacaggacgttcttgagttaccagtggattagagtcccatgaatgaagaccccccccaccccggttctccttaagcataggtcatacctccagaatagccagccacatcactatccccatgtaacatcagtctcctcaaaatggcgtgaggtcactagaaagaccttatactctcctctccttctcagagatgccctccattcacttaagtccctgttctcacccctgaacaagacacctaattaaccggcccactcacctcaattacaaacaccaaaatcgtcctggaagcatgaattacaggacagcaagtcttcctgccctctgcacccttgagaaacccccagtgccttgtatgaagcccaccccacatggcccacagtccctgtgctggccaaggctcccagaaaattctctattttttaaagtaataacttccccccctttggggggatccccaaatttggagaccccattctagaacactggggagttcaaattccagagagaatatatattatatataatccccaattccccatgcttccaagccctacaatctctagaagaccccaaatttctaattcccaggacttcccctacccaagtcacagaatcttcaaatccccagggaatcccaaacttaagataccaatcccaaaccctcaggaaatcccccaacacaaggtccttaggaccgggaggaaggaacctgttgccaggagaacatcccaggctctcagggcatctcaaacctgactcccaggcaccaggagaccccaaacagaaagtcccatctttggaacaaggataggactctaatacccttagtccatggatctttaatttcccaacctccaaactccatgggccccaccctcaagggaacccccaagatccaaatctctgataactaatatgtgcagggccccagggctctaacaggaccccaaatcatggagtccctacttcaatctaccttctggtcacaggtccaagacactaaatctgagtcattggccccaaaggacttcacagcacctgggccagactaacagcctgagggagaacctgagggccccgtgggtccagagcagacctggggccctgaccaccaaggacagctcacgactgccccttcactgcatgtccctaaactcagcatgactcctgtcctcttcaataaagacgtttctatggcaaaaaaaaaaaaaaaaaaaaaaaaaaaaa

SEQ ID NO: 57 - protein, 267 aa, Rattus sp.
LKGARPRVVNSTCSDFNHGSALHIAASNLCLGAAKCLLEHGANPALRNRKGQVPAEVVPDPMDMSLDKAEAALVAKELRTLLEEAVPLSCTLPKVTLPNYDNVPGNLMLSALGLRLGDRVLLDGQKTGTLRFCGTTEFASGQWVGVELDEPEGKNDGSVGGVRYFICPPKQGLFASVSKVSKAVDAPPSSVTSTPRTPRMDFSRVTGKGRREHKGKKKSPSSPSLGSLQQREGAKAEVGDQVLVAGQNRDCAFLWEDRLCSRLLVWH

SEQ ID NO: 58 - DNA, 2929 bp, Rattus sp.
  CDS (1)..(810)
gctgactctacctctagatgggctgaggccctcagagaaatctctggtcgcttagctgaaatgcctgcagatagtggataccctgcataccttggtgcccgactggcttctttctatgagcgagcaggcagagtgaaatgtcttggaaaccctgagagagaagggagtgtcagcattgtaggagcagtttctccacctggtggtgatttttctgatccagtcacatctgctactctgggtattgttcaggtgttctggggcttggataagaagctagctcagcgcaagcacttcccgtccgtcaactggctcattagctacagcaagtacatgcgcgccctggacgagtactatgacaaacacttcacagagttcgtgcctctgaggaccaaagctaaggagattctgcaggaagaggaggatctggcggaaatcgtgcagctcgtgggaaaggcgtctttagcagagacagataaaatcaccctggaggtagcaaaacttatcaaagatgacttcctacaacaaaatgggtacactccttatgacaggttctgtccattctataagacggtggggatgctgtccaacatgatttcattctatgatatggcccgccgggctgtggagaccaccgcccagagtgacaataagatcacatggtccattatccgtgagcacatgggggagattctctataaactttcctccatgaaattcaaggatccagtgaaggatggcgaggcaaagatcaaggccgactacgcacagcttcttgaagatatgcagaacgcattccgtagcctggaagattagaactgtgacttctctcctcctcttccgcagctcatatgtgtatattttcctgaatttctcatctccaaccctttgcttccatattgtgcagctttgagactagtgcctcgtgcgttctcgttcattttgctgtttctttggtaggtcttataaaacacacattcctgtgctccgctgtctgaaggagctcctgacctttgtctgaagtggtgaatgtagtgcatatgatacacagtgtaacatacacattgtaacatatacgttctgtaaacttgtatgtaaggtgactaccccttccctcctctccagtaaactgtaaacaggactactgcatgtgctctattggggatggaaggccagatctccataccgtggacaggtacataaggaaactagaccacttgcaacttagtgtttgttgagtaaccattttgcaggaagtatttccatttaaaaaacaaaagattaatgttccaattatttgtagcttccccagtatcaatcaggactgtttgtggcgcacttgggaactattttgttttcctaacagacgtttgcaaggctgaacgtaatagataaatcagttccctctgaaagtgtgaaagtaaaaagagagctaggtggtcagacttaaattgacatcgtcttgtttaagcatattttatttcactgagagatttaatatcaaggacttttatatactcaattactaggaaatctttttttaagtacaatttaaaaatcattgaaaatgtgatccacatcatagccattttccttatatttagtcagatgagctcagagtggggagggtgtgggttagaataccacaaggacacgcagcagtgcctgcaggcagtgtggccgggggccagagcggcattgttttcacgaggtacgtgtgtggcgtgtgtgtttgcttgttgacactctgaaaacagcaagcttaccagttccaggaaatattttgttttctttcactggctcagaaagctcctcaaagtacctggtccctgaagcttcctatctgttaatagagacgagagaggttcttaaatttaactggtgacaaaacaaaaagaaaaaaaagatcgatttttgtcttgctgttttggtgtgtttaaataataattccatatttgcataacgaggctcgcttctgagagcttggagatcgtgctccctcttcactctccggggtgataatgctggcgccatgctacctcttcaggaggggaaggggattgaacatggctaacactctcaagtacacaagcgtaacgacaaagtatttattttaagccttggtatgttgtttaaattattaggtggtgcatttcttatggtcttttgggtagacatagtatacacttcagatgtaatgtgtaaatccttgctagtgcatgtctacacgatagactgctattcaagaaggatattcttccacataacaatttaaaaactattaaatcagatatggattatgcaatgacttgttgagaggtggattaacggtgctgcttaatcagtttgcttccaatatggcttcgtatccagaagccctgactagtggagatgagaaagatttcaaaacctgtctgcctacacctaccagcaacctaggcttgtgatcagaatgaatgatcccaagaaactacttgaccaagtgtgttttgttgtcctggatttgagatgtgcgttcttcctccctctgagactgttgatgtatgagtgtgaagaagttacagaaacaacgctcagattttcacggtaactttccctctgcccacactgtagagtttcagattgttcactgatagtgcttctttcgtaaggatgtgttaaaatatagcagtctttttaaaagattatgcagttctctatttattgtgctgtgcctggtcctaagtgcagccggttaaacaagtttcatatgtatttttccagtgttaaatctcatacctatgccctttggaaagctccatcctgaacaatgaatagaagaggctatataaattgcctccttatccttaagatttcactatctttatgttaagagtaatgtataattattaaaatctatgaaaaataaaaagtggatttaaattaagagatc

SEQ ID NO: 59 - protein, 270 aa, Rattus sp.
ADSTSRWAEALREISGRLAEMPADSGYPAYLGARLASFYERAGRVKCLGNPEREGSVSIVGAVSPPGGDFSDPVTSATLGIVQVFWGLDKKLAQRKHFPSVNWLISYSKYMRALDEYYDKHFTEFVPLRTKAKEILQEEEDLAEIVQLVGKASLAETDKITLEVAKLIKDDFLQQNGYTPYDRFCPFYKTVGMLSNMISFYDMARRAVETTAQSDNKITWSIIREHMGEILYKLSSMKFKDPVKDGEAKIKADYAQLLEDMQNAFRSLED

SEQ ID NO: 60 - DNA, 1489 bp, Rattus sp.
  CDS (1)..(1053)
gcacggctcccggccccggagcatgcgcgacagcagcccctcctctccggccctgagcccggatcgtccgcccgggttccagttcccggcgtggccagtaggcggcagccgcgaggcggcaagccacccagcggggacggcctggagtcgggcccctctccacgcccccttctccacgcgcgcggggaggcagggctccaccgccagtctggaagggttccacatacaggaacggcctacttcgcagatgagcccaccgaggctcaggctccgggcggattctgcgtgtcaccctcgctccttggggtccgctggccggcctgtgccacccggacgcccggctcactgcctctgtctcccccatcagcgcagccccggacgctatggcccacccctccagctggcccctcgagtaggatggtagcacgtaaccaggtggcagccgacaatgcgatctccccggcatcagagccccgacggcggccagagccatcctcgtcctcgtcttcgtcctcgccggcggccccggcgcgtccccggccctgcccggtggtcccggccccggctccgggcgacactcacttccgcaccttccgctcccactctgattaccggcgcatcacgcggaccagcgctctcctggacgcctgcggcttctactggggacccctgagcgtgcatggggcgcacgaacggctgcgtgccgagcccgtgggcaccttcttggtgcgcgacagtcgccagcggaactgcttcttcgcgctcagcgtgaagatggcttcgggccccacgagcattcgtgtgcacttccaggccggccgcttccacctggacggcagccgcgagaccttcgactgcctcttcgagctgctggagcactacgtggcggcgccgcgccgcatgttgggggccccactgcgccagcgccgcgtgcggccgctgcaggagctgtgtcgccagcgcatcgtggccgccgtgggtcgcgagaacctggcacgcatccctcttaacccggtactccgtgactacctgagttccttccccttccagatctgaccggctgccgccgtgcccgcagcattaagtgggagcgccttattatttcttattattaattattattatttttctggaaccacgtgggagccctccccgcctaggtcggagggagtgggtgtggagggtgagatgcctcccacttctggctggagaccttatcccgcctctcggggggcctcccctcctggtgctccctcccggtccccctggttgtagcagcttgtgtctggggccaggacctgaactccacgcctacctctccatgtttacatgttcccagtatctttgcacaaaccaggggtgggggagggtctctggcttcatttttctgctgtgcagaatattctattttatatttttacatccagtttagataataaactttattatgaaagttttttttttaaagaaaaaaaaaaaaaaaaaaaaa

SEQ ID NO: 61 - protein, 351 aa, Rattus sp.
ARLPAPEHARQQPLLSGPEPGSSARVPVPGVASRRQPRGGKPPSGDGLESGPSPRPLLHARGEAGLHRQSGRVPHTGTAYFADEPTEAQAPGGFCVSPSLLGVRWPACATRTPGSLPLSPPSAQPRTLWPTPPAGPSSRMVARNQVAADNAISPASEPRRRPEPSSSSSSSSPAAPARPRPCPVVPAPAPGDTHFRTFRSHSDYRRITRTSALLDACGFYWGPLSVHGAHERLRAEPVGTFLVRDSRQRNCFFALSVKMASGPTSIRVHFQAGRFHLDGSRETFDCLFELLEHYVAAPRRMLGAPLRQRRVRPLQELCRQRIVAAVGRENLARIPLNPVLRDYLSSFPFQI

SEQ ID NO: 62 - DNA, 1194 bp, Rattus sp.
  CDS (130)..(765)
ggcacggctcccggccccggagcatgcgcgacagcagccccggaacccccagccgcggcgccccgcgtcccgccgccagcgcagccccggacgctatggcccacccctccagctggcccctcgagtaggatggtagcacgtaaccaggtggcagccgacaatgcgatctccccggcatcagagccccgacggcggccagagccatcctcgtcctcgtcttcgtcctcgccggcggccccggcgcgtccccggccctgcccggtggtcccggccccggctccgggcgacactcacttccgcaccttccgctcccactctgattaccggcgcatcacgcggaccagcgctctcctggacgcctgcggcttctactggggacccctgagcgtgcatggggcgcacgaacggctgcgtgccgagcccgtgggcaccttcttggtgcgcgacagtcgccagcggaactgcttcttcgcgctcagcgtgaagatggcttcgggccccacgagcattcgtgtgcacttccaggccggccgcttccacctggacggcagccgcgagaccttcgactgcctcttcgagctgctggagcactacgtggcggcgccgcgccgcatgttgggggccccactgcgccagcgccgcgtgcggccgctgcaggagctgtgtcgccagcgcatcgtggccgccgtgggtcgcgagaacctggcacgcatccctcttaacccggtactccgtgactacctgagttccttccccttccagatctgaccggctgccgccgtgcccgcagcattaagtgggagcgccttattatttcttattattaattattattatttttctggaaccacgtgggagccctccccgcctaggtcggagggagtgggtgtggagggtgagatgcctcccacttctggctggagaccttatcccgcctctcggggggcctcccctcctggtgctccctcccggtccccctggttgtagcagcttgtgtctggggccaggacctgaactccacgcctacctctccatgtttacatgttcccagtatctttgcacaaaccaggggtgggggagggtctctggcttcatttttctgctgtgcagaatattctattttatatttttacatccagtttagataataaactttattatgaaagttttttttttaaaaaaaaaaaaaaaaaa

SEQ ID NO: 63 - protein, 212 aa, Rattus sp.
MVARNQVAADNAISPASEPRRRPEPSSSSSSSSPAAPARPRPCPVVPAPAPGDTHFRTFRSHSDYRRITRTSALLDACGFYWGPLSVHGAHERLRAEPVGTFLVRDSRQRNCFFALSVKMASGPTSIRVHFQAGRFHLDGSRETFDCLFELLEHYVAAPRRMLGAPLRQRRVRPLQELCRQRIVAAVGRENLARIPLNPVLRDYLSSFPFQI

SEQ ID NO: 64 - DNA, 600 bp, Rattus sp.
  CDS (52)..(336)
cttccaaagactgcagcgcctcagggcccaggtttcaacagattcttcaaaatgccatcccaaatggagcatgccatggaaaccatgatgcttacatttcacaggtttgcaggggaaaaaaactacttgacaaaggaggacctgagagtgctcatggaaagggagttccctgggtttttggaaaatcaaaaggaccctctggctgtggacaaaataatgaaagacctggaccagtgccgagatggaaaagtgggcttccagagctttctatcactagtggcggggctcatcattgcatgcaatgactattttgtagtacacatgaagcagaagaagtaggccaactggagccctggtacccacaccttgatgcgtcctctcccatggggtcaactgaggaatctgccccactgcttcctgtgagcagatcaggacccttaggaaatgtgcaaataacatccaactccaattcgacaagcagagaaagaaaagttaatccaatgacagaggagctttcgagttttatattgtttgcatccggttgccctcaataaagaaagtctttttttttaagttccgaaaaaaaaaaaaaaaaaaaaa

SEQ ID NO: 65 - protein, 95 aa, Rattus sp.
MPSQMEHAMETMMLTFHRFAGEKNYLTKEDLRVLMEREFPGFLENQKDPLAVDKIMKDLDQCRDGKVGFQSFLSLVAGLIIACNDYFVVHMKQKK

SEQ ID NO: 66 - DNA, 639 bp, Rattus sp.
  CDS (1)..(636)
atggcgtacgcctatctcttcaagtacatcatcatcggcgacacaggtgttggtaaatcgtgcttattgctacagtttacagacaagaggtttcagccggtgcatgacctcacaattggtgtagagtttggtgctcgaatgataaccattgatgggaaacagataaaactccagatctgggatacagcagggcaggagtcctttcgttctatcacaaggtcatattacagaggtgcagcgggggctttactagtgtatgatattacaaggagagacacgttcaaccacttgacaacctggttagaagacgcccgtcagcattccaattccaacatggtcatcatgcttattggaaataaaagtgacttagaatctaggagagaagtgaaaaaggaagaaggtgaagcttttgcacgagagcatggacttatcttcatggaaacttctgccaagactgcttctaatgtagaggaggcatttattaacacagcaaaagaaatttatgaaaaaatccaagaaggggtctttgacattaataatgaggcaaacggcatcaaaattggccctcagcatgctgctaccaatgcatctcacggaggcaaccaaggagggcagcaggcagggggaggctgctgctga

SEQ ID NO: 67 - protein, 212 aa, Rattus sp.
MAYAYLFKYIIIGDTGVGKSCLLLQFTDKRFQPVHDLTIGVEFGARMITIDGKQIKLQIWDTAGQESFRSITRSYYRGAAGALLVYDITRRDTFNHLTTWLEDARQHSNSNMVIMLIGNKSDLESRREVKKEEGEAFAREHGLIFMETSAKTASNVEEAFINTAKEIYEKIQEGVFDINNEANGIKIGPQHAATNASHGGNQGGQQAGGGCC

SEQ ID NO: 68 - DNA, 816 bp, Rattus sp.
  CDS (1)..(813)
atggtgctgctcaaggaatatcgggtcatcctgcctgtgtctgtagatgagtatcaagtggggcagctgtactctgtggctgaagccagtaaaaatgaaactggtggtggggaaggtgtggaggtcctggtgaacgagccctacgagaaggatgatggcgagaaaggccagtacacacacaagatctaccacttacagagcaaagttcccacgtttgttcgaatgctggccccagaaggcgccctgaatatacatgagaaagcctggaatgcctacccttactgcagaaccgttattacaaatgagtacatgaaggaagactttctcattaaaattgaaacctggcacaagccagaccttggcacccaggagaatgtgcataaactggagcctgaggcatggaaacatgtggaagctatatatatagacatcgctgatcgaagccaagtacttagcaaggattacaaggcagaggaagacccagcaaaatttaaatctatcaaaacaggacgaggaccattgggcccgaattggaagcaagaacttgtcaatcagaaggactgcccatatatgtgtgcatacaaactggttactgtcaagttcaagtggtggggcttgcagaacaaagtggaaaactttatacataagcaagagaagcgtctgtttacaaactttcacaggcagctgttctgttggcttgataaatgggttgatctgactatggatgacattcggaggatggaagaagagacgaagagacagctggatgagatgagacaaaaggaccccgtgaaaggaatgacagcagatgactag

SEQ ID NO: 69 - DNA, 2263 bp, Simian sp.
cgctctcctcctcccctttctctagcagtagccttcttaatgtagtttaatggctttacaaagaaagccaggcagaggagcacttctcagtggctgtggtcggaccatgacctagctgaccatgaacttggaagggcttgaaatgatagcagttctgatcgtcattgtgctttttgttaaattattggaacagtttgggctgattgaagcaggtttagaagacagcgtggaagatgaactggagatggccactgtcaggcatcggcctgaggcccttgagcttctggaagcccagagcaaatttaccaagaaagagcttcagatcctttacagaggatttaagaacgaatgccccagtggtgttgttaatgaagaaaccttcaaagagatttactcgcagttctttccacagggagactctacaacatatgcacattttctgttcaatgcgtttgatacggaccacaatggagctgtgagtttcgaggatttcatcaaaggtctttccattttgctccgggggacagtacaagaaaaactcaattgggcatttaatctgtatgatataaataaagatggctacatcactaaagaggaaatgcttgatataatgaaagcaatatacgacatgatgggtaaatgtacatatcctgtcctcaaagaagatgcacccagacaacacgtcgaaacattttttcagaaaatggacaaaaataaagatggggttgttaccatagatgagttcattgaaagctgccaaaaagatgaaaacataatgcgctccatgcagctctttgaaaatgtgatttaacttgtcaactagatcctgaatccaacagacaaatgtgaactattctaccacccttaaagtcggagctaccacttttagcatagattgctcagcttgacactgaagcatattatgcaaacaagctttgttttaatataaagcaatccccaaaagatttgagtttctcagttataaatttgcatcctttccataatgccactgagttcatgggatgttctaactcatttcatactctgtgaatattcaaaagtaatagaatctggcatatagttttattgattccttagccatgggattattgaggctttcacatatcagtgattttaaaataccagtgttttttgctactcatttgtatgtattcagtcctaggattttgaatggttttctaatatactgacatctgcatttaatttccagaaattaaattaattttcatgtctgaatgctgtaattccatttatatactttaagtaaacaaataagattactacaattaaacacatagttccagtttctatggccttcacttcccaccttctattagaaattaattttatctggtatttttaaacatttaaaaatttatcatcagatatcagcatatgcctaattatgcctaatgaaacttaataagcatttaattttccatcatacattatagtcaaggcctatatactatatataattttggatttgtttaatcttacaggctgttttccattgtatcatcaagtggaagttcaagacggcatcaaacaaaacaaggatgtttacagacatatgcaaagggtcaggatatctatcctccagtatatgttaatgcttaataacaagtaatcctaacagcattaaaggccaaatctgtcctctttcccctgacttccttacagcatgtttatattacaagccattcagggacaaagaaaccttgactaccccactgtctactaggaacaaacaaacagcaagcaaaattcactttgaaagcaccagtggttccattacattgacaactactaccaagattcagtagaaaataagtgctcaacaactaatccagattacaatatgatttagtgcatcataaaattccaacaattcagattatttttaatcacctcagccacaactgtaaagttgccacattactaaagacacacacatcgtccctgttttgtagaaatatcacaaagaccaagaggctacagaaggaggaaatttgcaactgtctttgcaacaataaatcaggtatctattctggtgtagagataggatgttgaaagctgccctgctatcaccagtgtagaaattaagagtagtacaatacatgtacactgaaatttgccatcgcgtgtttgtgtaaactcaatgtgcacattttgtatttcaaaaagaaaaaataaaagcaaaataaaatgtttataactctaaaaaaaaaaaaaaaaaaaa

SEQ ID NO: 70 - protein, 229 aa, Simian sp.
MNLEGLEMIAVLIVIVLFVKLLEQFGLIEAGLEDSVEDELEMATVRHRPEALELLEAQSKFTKKELQILYRGFKNECPSGVVNEETFKEIYSQFFPQGDSTTYAHFLFNAFDTDHNGAVSFEDFIKGLSILLRGTVQEKLNWAFNLYDINKDGYITKEEMLDIMKAIYDMMGKCTYPVLKEDAPRQHVETFFQKMDKNKDGVVTIDEFIESCQKDENIMRSMQLFENVI

SEQ ID NO: 71 - DNA, 2259 bp, Simian sp.
gtcgacagacgcccctggccggtggactcctgagtcttactcctgcaccctgcgtccccagacatgaatgtgaggagagtggaaagcatttcggctcagctggaggaggccagctccacaggcggtttcctgtatgctcagaacagcaccaagcgcagcattaaagagcggctcatgaagctcttgccctgctcagctgccaaaacatcgtctcctgctattcaaaacagcgtggaagatgaactggagatggccactgtcaggcatcggcctgaggcccttgagcttctggaagcccagagcaaatttaccaagaaagagcttcagatcctttacagaggatttaagaacgaatgccccagtggtgttgttaatgaagaaaccttcaaagagatttactcgcagttctttccacagggagactctacaacatatgcacattttctgttcaatgcgtttgatacggaccacaatggagctgtgagtttcgaggatttcatcaaaggtctttccattttgctccgggggacagtacaagaaaaactcaattgggcatttaatctgtatgatataaataaagatggctacatcactaaagaggaaatgcttgatataatgaaagcaatatacgacatgatgggtaaatgtacatatcctgtcctcaaagaagatgcacccagacaacacgtcgaaacattttttcagaaaatggacaaaaataaagatggggttgttaccatagatgagttcattgaaagctgccaaaaagatgaaaacataatgcgctccatgcagctctttgaaaatgtgatttaacttgtcaactagatcctgaatccaacagacaaatgtgaactattctaccacccttaaagtcggagctaccacttttagcatagattgctcagcttgacactgaagcatattatgcaaacaagctttgttttaatataaagcaatccccaaaagatttgagtttctcagttataaatttgcatcctttccataatgccactgagttcatgggatgttctgactcatttcatactctgtgaatattcaaaagtaatagaatctggcatatagttttattgattccttagccatgggattattgaggctttcacatatcagtgattttaaaataccagtgttttttgctactcatttgtatgtattcagtcctaggattttgaatggttttctaatatactgacatctgcatttaatttccagaaattaaattaattttcatgtctgaatgctgtaattccatttatatactttaagtaaacaaataagattactacaattaaacacatagttccagtttctatggccttcacttcccaccttctattagaaattaattttatctggtatttttaaacatttaaaaatttatcatcagatatcagcatatgcctaattatgcctaatgaaacttaataagcatttaattttccatcatacattatagtcaaggcctatatactatatataattttggatttgtttaatcttacaggctgttttccattgtatcatcaagtggaagttcaagacggcatcaaacaaaacaaggatgtttacagacatatgcaaagggtcaggatatctatcctccagtatatgttaatgcttaataacaagtaatcctaacagcattaaaggccaaatctgtcctctttcccctgacttccttacagcatgtttatattacaagccattcagggacaaagaaaccttgactaccccactgtctactaggaacaaacaaacagcaagcaaaattcactttgaaagcaccagtggttccattacattgacaactactaccaagattcagtagaaaataagtgctcaacaactaatccagattacaatatgatttagtgcatcataaaattccaacaattcagattatttttaatcacctcagccacaactgtaaagttgccacattactaaagacacacacatcgtccctgttttgtagaaatatcacaaagaccaagaggctacagaaggaggaaatttgcaactgtctttgcaacaataaatcaggtatctattctggtgtagagataggatgttgaaagctgccctgctatcaccagtgtagaaattaagagtagtacaatacatgtacactgaaatttgccatcgcgtgtttgtgtaaactcaatgtgcacattttgtatttcaaaaagaaaaaataaaagcaaaataaaatgttaaaaaaaaaaaaaaaaaaaa

SEQ ID NO: 72 - protein, 250 aa, Simian sp.
MNVRRVESISAQLEEASSTGGFLYAQNSTKRSIKERLMKLLPCSAAKTSSPAIQNSVEDELEMATVRHRPEALELLEAQSKFTKKELQILYRGFKNECPSGVVNEETFKEIYSQFFPQGDSTTYAHFLFNAFDTDHNGAVSFEDFIKGLSILLRGTVQEKLNWAFNLYDINKDGYITKEEMLDIMKAIYDMMGKCTYPVLKEDAPRQHVETFFQKMDKNKDGVVTIDEFIESCQKDENIMRSMQLFENVI